SEQUENCE LISTING (195 sequences)

SEQ ID NO: 1 - protein, 993 aa, Homo sapiens
  REGION 1..993: note = MISC_FEATURE - HUMAN Receptor-type tyrosine-protein kinase FLT3, UniprotKBAccession Number: P36888-1
  DOMAIN 27..543: note = Extracellular Domain
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 2 - protein, 7 aa, synthetic construct
  REGION 1..7: note = Epitope 1
KSSSYPM

SEQ ID NO: 3 - protein, 7 aa, synthetic construct
  REGION 1..7: note = Epitope 2
SQGESCK

SEQ ID NO: 4 - protein, 4 aa, synthetic construct
  REGION 1..4: note = Epitope 3
DGYP

SEQ ID NO: 5 - protein, 119 aa, synthetic construct
  REGION 1..119: note = Clone 20D9, heavy chain variable region without a signal seqeunce
DVMLVESGGGLVQPGRSLKLSCVASGFTFNNYWMTWIRQAPGKGLEWIASITKTGGGTYYPDSVKGRFTISRDNAKSTLYLQMNSLRSEDTATYYCTTLQQLGVMDAWGQGASVTVSSA

SEQ ID NO: 6 - protein, 108 aa, synthetic construct
  REGION 1..108: note = Clone 20D9, light chain variable region without a signal seqeunce
DVQMTQSPSVLSASVGDRITLNCKASQNINKELNWYQQKLGEAPKLLIYNTNNLQTGIPSRFSGSGSGTDYTLTISSLQPEDVATYFCFQHKSWPLTFGSGTKLEIKR

SEQ ID NO: 7 - protein, 119 aa, synthetic construct
  REGION 1..119: note = Clone 2F12, heavy chain variable region without a signal seqeunce
EVKLEESGGGLVQPGGSMKLSCVASGFTFSNYWMNWVRQSPEKGLEWVAQIRLKSDNYATHYAESVKGRFTISRDDSKDSVYLQMNNLRAEDTGIYYCAVSLARSDYWGQGTTLTVSSA

SEQ ID NO: 8 - protein, 108 aa, synthetic construct
  REGION 1..108: note = Clone 2F12, light chain variable region without a signal seqeunce
DVVMTQSPPSLSASLGDKVTITCQASQNINKYIAWYQQKPGKAPRLLIRYTSTLESGTPSRFSGSGSGRDFSFSISNVESEDIASYYCLQYVNLPRTFGGGTKLELKR

SEQ ID NO: 9 - protein, 114 aa, synthetic construct
  REGION 1..114: note = Clone 4B12, heavy chain variable region without a signal sequence
QVQLQQSGPGLVQPSQTLSLTCTVSGFSLTSYNVNWVRQPPGKGLEWIGAMWRGGGTDYNPALKSRLSISRDTSKSQVFLKMNRLQTEDTAIYFCTRGFGDYWGQGVMVTVSSA

SEQ ID NO: 10 - protein, 113 aa, synthetic construct
  REGION 1..113: note = Clone 4B12, light chain variable region without a signal seqeunce
DIQMTQTPLSLSVAIGQSASISCKSSQSLKYSDGKTYLNWVFQSPGQSPKRLIYQVSKLDSGVPDRFSGTGSETDFTLKISRVEAEDLGVYYCCQGSYSPSTFGAGTKLELKR

SEQ ID NO: 11 - protein, 119 aa, synthetic construct
  REGION 1..119: note = Clone 27E7, heavy chain variable region without a signal sequence
EVKLEESGGDLVQPGGSMKLSCVASGFTFRNYWMNWVRQSPEKGLEWVAQIRLKSDNYATHYAESVKGRFTMSRDDSKSSVYLQMNTLRAEDTGIYYCTVSLARSDYWGQGTTLTVSSA

SEQ ID NO: 12 - protein, 113 aa, synthetic construct
  REGION 1..113: note = Clone 27E7, light chain variable region without a signal seqeunce
DVVMTQSPLTLSVTIGQPASISCKSSQSLLYSDGKTYLNWLLQRPGQSPKRLIYLVSKLDSGVPDRFTGSGSGTDFTLKISRVEAEDLGVYYCWQGTHFPYTFGGGTKLELKR

SEQ ID NO: 13 - protein, 120 aa, synthetic construct
  REGION 1..120: note = Clone 29H1, heavy chain variable region without a signal seqeunce
EVKLEESGGGLVQPGGSMKFFCVASGFTFSNYWMNWVRQSPEKGLEWVAQIKLKSDNYATRYAESVKGRFTISRDDSKSSVNLQMNNLRAEDTGIYYCTVSLARSDYWGQGTTLTVSSAS

SEQ ID NO: 14 - protein, 113 aa, synthetic construct
  REGION 1..113: note = Clone 29H1, light chain variable region without a signal seqeunce
DVVMTQTPLTLSVAIGQPASISCKSSQSLLHSDGKTYLNWLLHRPGQSPKRLIYLVSKLDSGVPDRFTGSGSGTDFTLKISRVEAEDLGVYYCWQGTHFPYTFGGGTKLEIKR

SEQ ID NO: 15 - protein, 121 aa, synthetic construct
  REGION 1..121: note = Clone 30B12, heavy chain variable region without a signalseqeunce
QVQLKVSGPGLVQPSQTLSLTCAVSGFSLSNYHVSWVRQPPGKGLEWIAAISSGGSTYYNSPLKSRLSISRDTSKSQVFLKMNSLQTEDTAIYFCTGEDGYTFGNVMDAWGQGASVTVSSA

SEQ ID NO: 16 - protein, 113 aa, synthetic construct
  REGION 1..113: note = Clone 30B12, light chain variable region without a signalseqeunce
DVVMTQTPLTLSVTIGQPASISCKSSQSLLYSDGKTYLNWLLQRPGQSPKRLIYLVSKLDSGVPDRFTGSGSGTDFTLKISRVEAEDLGVYFCWQGTHFPYTFGGGTKLERKR

SEQ ID NO: 17 - protein, 104 aa, synthetic construct
  REGION 1..104: note = Clone 19H5, heavy chain variable region without a signal seqeunce
MVQPSQTLSLTCTVSGFSLTSYNVNWVRQPPGKGLEWIGAMWRGGGTDYNPALKSRLSISRDTSKSQVFLKMNRLQTEDTAIYFCTRGFGDYWGQGVMVTVSSA

SEQ ID NO: 18 - protein, 108 aa, synthetic construct
  REGION 1..108: note = Clone 19H5, light chain variable region without a signal seqeunce
DVQMTQSPSSMSASLGDRVTFTCQASQDIGNNLIYFQQKPGKSPRRMICCATNLAHGVPSRFSGSRSGSDYSLTLVSLESEDVADYHCLQYEHYPRTFGGGTKLELKR

SEQ ID NO: 19 - protein, 19 aa, synthetic construct
  REGION 1..19: note = signal sequence of the variable region of the heavy chain
MDWTWRILFLVAAATGAHS

SEQ ID NO: 20 - protein, 19 aa, synthetic construct
  REGION 1..19: note = signal sequence of the variable region of the light chain
MLPSQLIGFLLLWVPASRG

SEQ ID NO: 21 - protein, 5 aa, synthetic construct
  REGION 1..5: note = CDR-H1
NYHVS

SEQ ID NO: 22 - protein, 16 aa, synthetic construct
  REGION 1..16: note = CDR-H2
AISSGGSTYYNSPLKS

SEQ ID NO: 23 - protein, 12 aa, synthetic construct
  REGION 1..12: note = CDR-H3
EDGYTFGNVMDA

SEQ ID NO: 24 - protein, 16 aa, synthetic construct
  REGION 1..16: note = CDR-L1
KSSQSLLYSDGKTYLN

SEQ ID NO: 25 - protein, 7 aa, synthetic construct
  REGION 1..7: note = CDR-L2
LVSKLDS

SEQ ID NO: 26 - protein, 9 aa, synthetic construct
  REGION 1..9: note = CDR-L3
WQGTHFPYT

SEQ ID NO: 27 - protein, 5 aa, synthetic construct
  REGION 1..5: note = CDR-H1
NYWMN

SEQ ID NO: 28 - protein, 19 aa, synthetic construct
  REGION 1..19: note = CDR-H2
QIRLKSDNYATHYAESVKG

SEQ ID NO: 29 - protein, 7 aa, synthetic construct
  REGION 1..7: note = CDR-H3
SLARSDY

SEQ ID NO: 30 - protein, 11 aa, synthetic construct
  REGION 1..11: note = CDR-L1
QASQNINKYIA

SEQ ID NO: 31 - protein, 7 aa, synthetic construct
  REGION 1..7: note = CDR-L2
YTSTLES

SEQ ID NO: 32 - protein, 9 aa, synthetic construct
  REGION 1..9: note = CDR-L3
LQYVNLPRT

SEQ ID NO: 33 - protein, 5 aa, synthetic construct
  REGION 1..5: note = CDR-H1
SYNVN

SEQ ID NO: 34 - protein, 16 aa, synthetic construct
  REGION 1..16: note = CDR-H2
AMWRGGGTDYNPALKS

SEQ ID NO: 35 - protein, 5 aa, synthetic construct
  REGION 1..5: note = CDR-H3
GFGDY

SEQ ID NO: 36 - protein, 16 aa, synthetic construct
  REGION 1..16: note = CDR-L1
KSSQSLKYSDGKTYLN

SEQ ID NO: 37 - protein, 7 aa, synthetic construct
  REGION 1..7: note = CDR-L2
QVSKLDS

SEQ ID NO: 38 - protein, 9 aa, synthetic construct
  REGION 1..9: note = CDR-L3
CQGSYSPST

SEQ ID NO: 39 - protein, 5 aa, synthetic construct
  REGION 1..5: note = CDR-H1
SYNVN

SEQ ID NO: 40 - protein, 16 aa, synthetic construct
  REGION 1..16: note = CDR-H2
AMWRGGGTDYNPALKS

SEQ ID NO: 41 - protein, 5 aa, synthetic construct
  REGION 1..5: note = CDR-H3
GFGDY

SEQ ID NO: 42 - protein, 11 aa, synthetic construct
  REGION 1..11: note = CDR-L1
QASQDIGNNLI

SEQ ID NO: 43 - protein, 7 aa, synthetic construct
  REGION 1..7: note = CDR-L2
CATNLAH

SEQ ID NO: 44 - protein, 9 aa, synthetic construct
  REGION 1..9: note = CDR-L3
LQYEHYPRT

SEQ ID NO: 45 - protein, 5 aa, synthetic construct
  REGION 1..5: note = CDR-H1
NYWMT

SEQ ID NO: 46 - protein, 17 aa, synthetic construct
  REGION 1..17: note = CDR-H2
SITKTGGGTYYPDSVKG

SEQ ID NO: 47 - protein, 9 aa, synthetic construct
  REGION 1..9: note = CDR-H3
LQQLGVMDA

SEQ ID NO: 48 - protein, 11 aa, synthetic construct
  REGION 1..11: note = CDR-L1
KASQNINKELN

SEQ ID NO: 49 - protein, 7 aa, synthetic construct
  REGION 1..7: note = CDR-L2
NTNNLQT

SEQ ID NO: 50 - protein, 9 aa, synthetic construct
  REGION 1..9: note = CDR-L3
FQHKSWPLT

SEQ ID NO: 51 - protein, 5 aa, synthetic construct
  REGION 1..5: note = CDR-H1
NYWMN

SEQ ID NO: 52 - protein, 19 aa, synthetic construct
  REGION 1..19: note = CDR-H2
QIRLKSDNYATHYAESVKG

SEQ ID NO: 53 - protein, 7 aa, synthetic construct
  REGION 1..7: note = CDR-H3
SLARSDY

SEQ ID NO: 54 - protein, 16 aa, synthetic construct
  REGION 1..16: note = CDR-L1
KSSQSLLYSDGKTYLN

SEQ ID NO: 55 - protein, 7 aa, synthetic construct
  REGION 1..7: note = CDR-L2
LVSKLDS

SEQ ID NO: 56 - protein, 9 aa, synthetic construct
  REGION 1..9: note = CDR-L3
WQGTHFPYT

SEQ ID NO: 57 - protein, 5 aa, synthetic construct
  REGION 1..5: note = CDR-H1
NYWMN

SEQ ID NO: 58 - protein, 19 aa, synthetic construct
  REGION 1..19: note = CDR-H2
QIKLKSDNYATRYAESVKG

SEQ ID NO: 59 - protein, 7 aa, synthetic construct
  REGION 1..7: note = CDR-H3
SLARSDY

SEQ ID NO: 60 - protein, 16 aa, synthetic construct
  REGION 1..16: note = CDR-L1
KSSQSLLHSDGKTYLN

SEQ ID NO: 61 - protein, 7 aa, synthetic construct
  REGION 1..7: note = CDR-L2
LVSKLDS

SEQ ID NO: 62 - protein, 9 aa, synthetic construct
  REGION 1..9: note = CDR-L3
WQGTHFPYT

SEQ ID NO: 63 - protein, 448 aa, synthetic construct
  REGION 1..448: note = Humanized clone 1 of 20D9, heavy chain sequence without a signalpeptide
QVMLVESGGGVVQPGRSLRLSCAASGFTFNNYWMTWVRQAPGKGLEWIASITKTGGGTYYPDSVKGRFTISRDNAKSTLYLQMNSLRSEDTATYYCTTLQQLGVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 118 aa, synthetic construct
  REGION 1..118: note = Humanized clone 1 of 20D9, heavy chain variable region without asignal peptide
QVMLVESGGGVVQPGRSLRLSCAASGFTFNNYWMTWVRQAPGKGLEWIASITKTGGGTYYPDSVKGRFTISRDNAKSTLYLQMNSLRSEDTATYYCTTLQQLGVMDAWGQGASVTVSS

SEQ ID NO: 65 - protein, 5 aa, synthetic construct
  REGION 1..5: note = Humanized clone 1 of 20D9, heavy chain variable region, CDR-H1
NYWMT

SEQ ID NO: 66 - protein, 17 aa, synthetic construct
  REGION 1..17: note = Humanized clone 1 of 20D9, heavy chain variable region, CDR-H2
SITKTGGGTYYPDSVKG

SEQ ID NO: 67 - protein, 9 aa, synthetic construct
  REGION 1..9: note = Humanized clone 1 of 20D9, heavy chain variable region, CDR-H3
LQQLGVMDA

SEQ ID NO: 68 - protein, 214 aa, synthetic construct
  REGION 1..214: note = Humanized clone 1 of 20D9, light chain sequence without a signalpeptide
DIQMTQSPSVLSASVGDRVTINCRASQNINKELNWYQQKLGEAPKLLIYNTNNLQSGVPSRFSGSGSGTDYTLTISSLQPEDVATYFCFQHKSWPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 107 aa, synthetic construct
  REGION 1..107: note = Humanized clone 1 of 20D9, light chain variable region without asignal peptide
DIQMTQSPSVLSASVGDRVTINCRASQNINKELNWYQQKLGEAPKLLIYNTNNLQSGVPSRFSGSGSGTDYTLTISSLQPEDVATYFCFQHKSWPLTFGSGTKLEIK

SEQ ID NO: 70 - protein, 11 aa, synthetic construct
  REGION 1..11: note = Humanized clone 1 of 20D9, light chain variable region, CDR-L1
RASQNINKELN

SEQ ID NO: 71 - protein, 7 aa, synthetic construct
  REGION 1..7: note = Humanized clone 1 of 20D9, light chain variable region, CDR-L2
NTNNLQS

SEQ ID NO: 72 - protein, 9 aa, synthetic construct
  REGION 1..9: note = Humanized clone 1 of 20D9, light chain variable region, CDR-L3
FQHKSWPLT

SEQ ID NO: 73 - protein, 448 aa, synthetic construct
  REGION 1..448: note = Humanized clone 2 of 20D9, heavy chain sequence without a signalpeptide
QVMLVESGGGVVQPGRSLRLSCAASGFTFNNYWMTWVRQAPGKGLEWIASITKTGGGTYYPDSVKGRFTISRDNSKSTLYLQMNSLRAEDTATYYCTTLQQLGVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 118 aa, synthetic construct
  REGION 1..118: note = Humanized clone 2 of 20D9, heavy chain variable region without asignal peptide
QVMLVESGGGVVQPGRSLRLSCAASGFTFNNYWMTWVRQAPGKGLEWIASITKTGGGTYYPDSVKGRFTISRDNSKSTLYLQMNSLRAEDTATYYCTTLQQLGVMDAWGQGASVTVSS

SEQ ID NO: 75 - protein, 5 aa, synthetic construct
  REGION 1..5: note = Humanized clone 2 of 20D9, heavy chain variable region, CDR-H1
NYWMT

SEQ ID NO: 76 - protein, 17 aa, synthetic construct
  REGION 1..17: note = Humanized clone 2 of 20D9, heavy chain variable region, CDR-H2
SITKTGGGTYYPDSVKG

SEQ ID NO: 77 - protein, 9 aa, synthetic construct
  REGION 1..9: note = Humanized clone 2 of 20D9, heavy chain variable region, CDR-H3
LQQLGVMDA

SEQ ID NO: 78 - protein, 214 aa, synthetic construct; identical to SEQ ID NO: 68
  REGION 1..214: note = Humanized clone 2 of 20D9, light chain sequence without a signalpeptide

SEQ ID NO: 79 - protein, 107 aa, synthetic construct; identical to SEQ ID NO: 69
  REGION 1..107: note = Humanized clone 2 of 20D9, light chain variable region without asignal peptide

SEQ ID NO: 80 - protein, 11 aa, synthetic construct
  REGION 1..11: note = Humanized clone 2 of 20D9, light chain variable region, CDR-L1
RASQNINKELN

SEQ ID NO: 81 - protein, 7 aa, synthetic construct
  REGION 1..7: note = Humanized clone 2 of 20D9, light chain variable region, CDR-L2
NTNNLQS

SEQ ID NO: 82 - protein, 9 aa, synthetic construct
  REGION 1..9: note = Humanized clone 2 of 20D9, light chain variable region, CDR-L3
FQHKSWPLT

SEQ ID NO: 83 - protein, 448 aa, synthetic construct
  REGION 1..448: note = Humanized clone 3 of 20D9, heavy chain sequence without a signalpeptide
QVQLVESGGGVVQPGRSLRLSCAASGFTFNNYWMTWVRQAPGKGLEWIASITKTGGGTYYADSVKGRFTISRDNSKNTLYLQMNSLRAEDTAVYYCTTLQQLGVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 118 aa, synthetic construct
  REGION 1..118: note = Humanized clone 3 of 20D9, heavy chain variable region without asignal peptide
QVQLVESGGGVVQPGRSLRLSCAASGFTFNNYWMTWVRQAPGKGLEWIASITKTGGGTYYADSVKGRFTISRDNSKNTLYLQMNSLRAEDTAVYYCTTLQQLGVMDAWGQGTLVTVSS

SEQ ID NO: 85 - protein, 5 aa, synthetic construct
  REGION 1..5: note = Humanized clone 3 of 20D9, heavy chain variable region, CDR-H1
NYWMT

SEQ ID NO: 86 - protein, 17 aa, synthetic construct
  REGION 1..17: note = Humanized clone 3 of 20D9, heavy chain variable region, CDR-H2
SITKTGGGTYYADSVKG

SEQ ID NO: 87 - protein, 9 aa, synthetic construct
  REGION 1..9: note = Humanized clone 3 of 20D9, heavy chain variable region, CDR-H3
LQQLGVMDA

SEQ ID NO: 88 - protein, 214 aa, synthetic construct; identical to SEQ ID NO: 68
  REGION 1..214: note = Humanized clone 3 of 20D9, light chain sequence without a signalpeptide

SEQ ID NO: 89 - protein, 107 aa, synthetic construct; identical to SEQ ID NO: 69
  REGION 1..107: note = Humanized clone 3 of 20D9, light chain variable region without asignal peptide

SEQ ID NO: 90 - protein, 11 aa, synthetic construct
  REGION 1..11: note = Humanized clone 3 of 20D9, light chain variable region, CDR-L1
RASQNINKELN

SEQ ID NO: 91 - protein, 7 aa, synthetic construct
  REGION 1..7: note = Humanized clone 3 of 20D9, light chain variable region, CDR-L2
NTNNLQS

SEQ ID NO: 92 - protein, 9 aa, synthetic construct
  REGION 1..9: note = Humanized clone 3 of 20D9, light chain variable region, CDR-L3
FQHKSWPLT

SEQ ID NO: 93 - protein, 448 aa, synthetic construct
  REGION 1..448: note = Humanized clone 4 of 20D9, heavy chain sequence without a signalpeptide
QVQLVESGGGVVQPGRSLRLSCAASGFTFNNYWMTWIRQAPGKGLEWIASITKTGGGTYYADSVKGRFTISRDNSKNTLYLQMNSLRAEDTAVYYCTTLQQLGVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 118 aa, synthetic construct
  REGION 1..118: note = Humanized clone 4 of 20D9, heavy chain variable region without asignal peptide
QVQLVESGGGVVQPGRSLRLSCAASGFTFNNYWMTWIRQAPGKGLEWIASITKTGGGTYYADSVKGRFTISRDNSKNTLYLQMNSLRAEDTAVYYCTTLQQLGVMDAWGQGTLVTVSS

SEQ ID NO: 95 - protein, 5 aa, synthetic construct
  REGION 1..5: note = Humanized clone 4 of 20D9, heavy chain variable region, CDR-H1
NYWMT

SEQ ID NO: 96 - protein, 17 aa, synthetic construct
  REGION 1..17: note = Humanized clone 4 of 20D9, heavy chain variable region, CDR-H2
SITKTGGGTYYADSVKG

SEQ ID NO: 97 - protein, 9 aa, synthetic construct
  REGION 1..9: note = Humanized clone 4 of 20D9, heavy chain variable region, CDR-H3
LQQLGVMDA

SEQ ID NO: 98 - protein, 214 aa, synthetic construct; identical to SEQ ID NO: 68
  REGION 1..214: note = Humanized clone 4 of 20D9, light chain sequence without a signalpeptide

SEQ ID NO: 99 - protein, 107 aa, synthetic construct; identical to SEQ ID NO: 69
  REGION 1..107: note = Humanized clone 4 of 20D9, light chain variable region without asignal peptide

SEQ ID NO: 100 - protein, 11 aa, synthetic construct
  REGION 1..11: note = Humanized clone 4 of 20D9, light chain variable region, CDR-L1
RASQNINKELN

SEQ ID NO: 101 - protein, 7 aa, synthetic construct
  REGION 1..7: note = Humanized clone 4 of 20D9, light chain variable region, CDR-L2
NTNNLQS

SEQ ID NO: 102 - protein, 9 aa, synthetic construct
  REGION 1..9: note = Humanized clone 4 of 20D9, light chain variable region, CDR-L3
FQHKSWPLT

SEQ ID NO: 103 - protein, 448 aa, synthetic construct; identical to SEQ ID NO: 63
  REGION 1..448: note = Humanized clone 5 of 20D9, heavy chain sequence without a signalpeptide

SEQ ID NO: 104 - protein, 118 aa, synthetic construct; identical to SEQ ID NO: 64
  REGION 1..118: note = Humanized clone 5 of 20D9, heavy chain variable region without asignal peptide

SEQ ID NO: 105 - protein, 5 aa, synthetic construct
  REGION 1..5: note = Humanized clone 5 of 20D9, heavy chain variable region, CDR-H1
NYWMT

SEQ ID NO: 106 - protein, 17 aa, synthetic construct
  REGION 1..17: note = Humanized clone 5 of 20D9, heavy chain variable region, CDR-H2
SITKTGGGTYYPDSVKG

SEQ ID NO: 107 - protein, 9 aa, synthetic construct
  REGION 1..9: note = Humanized clone 5 of 20D9, heavy chain variable region, CDR-H3
LQQLGVMDA

SEQ ID NO: 108 - protein, 214 aa, synthetic construct
  REGION 1..214: note = Humanized clone 5 of 20D9, light chain sequence without a signalpeptide
DIQMTQSPSVLSASVGDRVTITCRASQNINKELNWYQQKLGKAPKLLIYNTNNLQSGVPSRFSGSGSGTDFTLTISSLQPEDFATYYCFQHKSWPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 107 aa, synthetic construct
  REGION 1..107: note = Humanized clone 5 of 20D9, light chain variable region without asignal peptide
DIQMTQSPSVLSASVGDRVTITCRASQNINKELNWYQQKLGKAPKLLIYNTNNLQSGVPSRFSGSGSGTDFTLTISSLQPEDFATYYCFQHKSWPLTFGSGTKLEIK

SEQ ID NO: 110 - protein, 11 aa, synthetic construct
  REGION 1..11: note = Humanized clone 5 of 20D9, light chain variable region, CDR-L1
RASQNINKELN

SEQ ID NO: 111 - protein, 7 aa, synthetic construct
  REGION 1..7: note = Humanized clone 5 of 20D9, light chain variable region, CDR-L2
NTNNLQS

SEQ ID NO: 112 - protein, 9 aa, synthetic construct
  REGION 1..9: note = Humanized clone 5 of 20D9, light chain variable region, CDR-L3
FQHKSWPLT

SEQ ID NO: 113 - protein, 448 aa, synthetic construct; identical to SEQ ID NO: 73
  REGION 1..448: note = Humanized clone 6 of 20D9, heavy chain sequence without a signalpeptide

SEQ ID NO: 114 - protein, 118 aa, synthetic construct; identical to SEQ ID NO: 74
  REGION 1..118: note = Humanized clone 6 of 20D9, heavy chain variable region without asignal peptide

SEQ ID NO: 115 - protein, 5 aa, synthetic construct
  REGION 1..5: note = Humanized clone 6 of 20D9, heavy chain variable region, CDR-H1
NYWMT

SEQ ID NO: 116 - protein, 17 aa, synthetic construct
  REGION 1..17: note = Humanized clone 6 of 20D9, heavy chain variable region, CDR-H2
SITKTGGGTYYPDSVKG

SEQ ID NO: 117 - protein, 9 aa, synthetic construct
  REGION 1..9: note = Humanized clone 6 of 20D9, heavy chain variable region, CDR-H3
LQQLGVMDA

SEQ ID NO: 118 - protein, 214 aa, synthetic construct; identical to SEQ ID NO: 108
  REGION 1..214: note = Humanized clone 6 of 20D9, light chain sequence without a signalpeptide

SEQ ID NO: 119 - protein, 107 aa, synthetic construct; identical to SEQ ID NO: 109
  REGION 1..107: note = Humanized clone 6 of 20D9, light chain variable region without asignal peptide

SEQ ID NO: 120 - protein, 11 aa, synthetic construct
  REGION 1..11: note = Humanized clone 6 of 20D9, light chain variable region, CDR-L1
RASQNINKELN

SEQ ID NO: 121 - protein, 7 aa, synthetic construct
  REGION 1..7: note = Humanized clone 6 of 20D9, light chain variable region, CDR-L2
NTNNLQS

SEQ ID NO: 122 - protein, 9 aa, synthetic construct
  REGION 1..9: note = Humanized clone 6 of 20D9, light chain variable region, CDR-L3
FQHKSWPLT

SEQ ID NO: 123 - protein, 448 aa, synthetic construct; identical to SEQ ID NO: 83
  REGION 1..448: note = Humanized clone 7 of 20D9, heavy chain sequence without a signalpeptide

SEQ ID NO: 124 - protein, 118 aa, synthetic construct; identical to SEQ ID NO: 84
  REGION 1..118: note = Humanized clone 7 of 20D9, heavy chain variable region without asignal peptide

SEQ ID NO: 125 - protein, 5 aa, synthetic construct
  REGION 1..5: note = Humanized clone 7 of 20D9, heavy chain variable region, CDR-H1
NYWMT

SEQ ID NO: 126 - protein, 17 aa, synthetic construct
  REGION 1..17: note = Humanized clone 7 of 20D9, heavy chain variable region, CDR-H2
SITKTGGGTYYADSVKG

SEQ ID NO: 127 - protein, 9 aa, synthetic construct
  REGION 1..9: note = Humanized clone 7 of 20D9, heavy chain variable region, CDR-H3
LQQLGVMDA

SEQ ID NO: 128 - protein, 214 aa, synthetic construct; identical to SEQ ID NO: 108
  REGION 1..214: note = Humanized clone 7 of 20D9, light chain sequence without a signalpeptide

SEQ ID NO: 129 - protein, 107 aa, synthetic construct; identical to SEQ ID NO: 109
  REGION 1..107: note = Humanized clone 7 of 20D9, light chain variable region without asignal peptide

SEQ ID NO: 130 - protein, 11 aa, synthetic construct
  REGION 1..11: note = Humanized clone 7 of 20D9, light chain variable region, CDR-L1
RASQNINKELN

SEQ ID NO: 131 - protein, 7 aa, synthetic construct
  REGION 1..7: note = Humanized clone 7 of 20D9, light chain variable region, CDR-L2
NTNNLQS

SEQ ID NO: 132 - protein, 9 aa, synthetic construct
  REGION 1..9: note = Humanized clone 7 of 20D9, light chain variable region, CDR-L3
FQHKSWPLT

SEQ ID NO: 133 - protein, 448 aa, synthetic construct; identical to SEQ ID NO: 93
  REGION 1..448: note = Humanized clone 8 of 20D9, heavy chain sequence without a signalpeptide

SEQ ID NO: 134 - protein, 118 aa, synthetic construct; identical to SEQ ID NO: 94
  REGION 1..118: note = Humanized clone 8 of 20D9, heavy chain variable region without asignal peptide

SEQ ID NO: 135 - protein, 5 aa, synthetic construct
  REGION 1..5: note = Humanized clone 8 of 20D9, heavy chain variable region, CDR-H1
NYWMT

SEQ ID NO: 136 - protein, 17 aa, synthetic construct
  REGION 1..17: note = Humanized clone 8 of 20D9, heavy chain variable region, CDR-H2
SITKTGGGTYYADSVKG

SEQ ID NO: 137 - protein, 9 aa, synthetic construct
  REGION 1..9: note = Humanized clone 8 of 20D9, heavy chain variable region, CDR-H3
LQQLGVMDA

SEQ ID NO: 138 - protein, 214 aa, synthetic construct; identical to SEQ ID NO: 108
  REGION 1..214: note = Humanized clone 8 of 20D9, light chain sequence without a signalpeptide

SEQ ID NO: 139 - protein, 107 aa, synthetic construct; identical to SEQ ID NO: 109
  REGION 1..107: note = Humanized clone 8 of 20D9, light chain variable region without asignal peptide

SEQ ID NO: 140 - protein, 11 aa, synthetic construct
  REGION 1..11: note = Humanized clone 8 of 20D9, light chain variable region, CDR-L1
RASQNINKELN

SEQ ID NO: 141 - protein, 7 aa, synthetic construct
  REGION 1..7: note = Humanized clone 8 of 20D9, light chain variable region, CDR-L2
NTNNLQS

SEQ ID NO: 142 - protein, 9 aa, synthetic construct
  REGION 1..9: note = Humanized clone 8 of 20D9, light chain variable region, CDR-L3
FQHKSWPLT

SEQ ID NO: 143 - protein, 448 aa, synthetic construct; identical to SEQ ID NO: 63
  REGION 1..448: note = Humanized clone 9 of 20D9, heavy chain sequence without a signalpeptide

SEQ ID NO: 144 - protein, 118 aa, synthetic construct; identical to SEQ ID NO: 64
  REGION 1..118: note = Humanized clone 9 of 20D9, heavy chain variable region without asignal peptide

SEQ ID NO: 145 - protein, 5 aa, synthetic construct
  REGION 1..5: note = Humanized clone 9 of 20D9, heavy chain variable region, CDR-H1
NYWMT

SEQ ID NO: 146 - protein, 17 aa, synthetic construct
  REGION 1..17: note = Humanized clone 9 of 20D9, heavy chain variable region, CDR-H2
SITKTGGGTYYPDSVKG

SEQ ID NO: 147 - protein, 9 aa, synthetic construct
  REGION 1..9: note = Humanized clone 9 of 20D9, heavy chain variable region, CDR-H3
LQQLGVMDA

SEQ ID NO: 148 - protein, 214 aa, synthetic construct
  REGION 1..214: note = Humanized clone 9 of 20D9, light chain sequence without a signalpeptide
DIQMTQSPSSLSASVGDRVTITCRASQSINKELNWYQQKPGKAPKLLIYNTNNLQSGVPSRFSGSGSGTDFTLTISSLQPEDFATYYCFQHKSWPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 107 aa, synthetic construct
  REGION 1..107: note = Humanized clone 9 of 20D9, light chain variable region without asignal peptide
DIQMTQSPSSLSASVGDRVTITCRASQSINKELNWYQQKPGKAPKLLIYNTNNLQSGVPSRFSGSGSGTDFTLTISSLQPEDFATYYCFQHKSWPLTFGQGTKLEIK

SEQ ID NO: 150 - protein, 11 aa, synthetic construct
  REGION 1..11: note = Humanized clone 9 of 20D9, light chain variable region, CDR-L1
RASQSINKELN

SEQ ID NO: 151 - protein, 7 aa, synthetic construct
  REGION 1..7: note = Humanized clone 9 of 20D9, light chain variable region, CDR-L2
NTNNLQS

SEQ ID NO: 152 - protein, 9 aa, synthetic construct
  REGION 1..9: note = Humanized clone 9 of 20D9, light chain variable region, CDR-L3
FQHKSWPLT

SEQ ID NO: 153 - protein, 448 aa, synthetic construct; identical to SEQ ID NO: 73
  REGION 1..448: note = Humanized clone 10 of 20D9, heavy chain sequence without a signalpeptide

SEQ ID NO: 154 - protein, 118 aa, synthetic construct; identical to SEQ ID NO: 74
  REGION 1..118: note = Humanized clone 10 of 20D9, heavy chain variable region without asignal peptide

SEQ ID NO: 155 - protein, 5 aa, synthetic construct
  REGION 1..5: note = Humanized clone 10 of 20D9, heavy chain variable region, CDR-H1
NYWMT

SEQ ID NO: 156 - protein, 17 aa, synthetic construct
  REGION 1..17: note = Humanized clone 10 of 20D9, heavy chain variable region, CDR-H2
SITKTGGGTYYPDSVKG

SEQ ID NO: 157 - protein, 9 aa, synthetic construct
  REGION 1..9: note = Humanized clone 10 of 20D9, heavy chain variable region, CDR-H3
LQQLGVMDA

SEQ ID NO: 158 - protein, 214 aa, synthetic construct; identical to SEQ ID NO: 148
  REGION 1..214: note = Humanized clone 10 of 20D9, light chain sequence without a signalpeptide

SEQ ID NO: 159 - protein, 107 aa, synthetic construct; identical to SEQ ID NO: 149
  REGION 1..107: note = Humanized clone 10 of 20D9, light chain variable region without asignal peptide

SEQ ID NO: 160 - protein, 11 aa, synthetic construct
  REGION 1..11: note = Humanized clone 10 of 20D9, light chain variable region, CDR-L1
RASQSINKELN

SEQ ID NO: 161 - protein, 7 aa, synthetic construct
  REGION 1..7: note = Humanized clone 10 of 20D9, light chain variable region, CDR-L2
NTNNLQS

SEQ ID NO: 162 - protein, 9 aa, synthetic construct
  REGION 1..9: note = Humanized clone 10 of 20D9, light chain variable region, CDR-L3
FQHKSWPLT

SEQ ID NO: 163 - protein, 448 aa, synthetic construct; identical to SEQ ID NO: 83
  REGION 1..448: note = Humanized clone 11 of 20D9, heavy chain sequence without a signalpeptide

SEQ ID NO: 164 - protein, 118 aa, synthetic construct; identical to SEQ ID NO: 84
  REGION 1..118: note = Humanized clone 11 of 20D9, heavy chain variable region without asignal peptide

SEQ ID NO: 165 - protein, 5 aa, synthetic construct
  REGION 1..5: note = Humanized clone 11 of 20D9, heavy chain variable region, CDR-H1
NYWMT

SEQ ID NO: 166 - protein, 17 aa, synthetic construct
  REGION 1..17: note = Humanized clone 11 of 20D9, heavy chain variable region, CDR-H2
SITKTGGGTYYADSVKG

SEQ ID NO: 167 - protein, 9 aa, synthetic construct
  REGION 1..9: note = Humanized clone 11 of 20D9, heavy chain variable region, CDR-H3
LQQLGVMDA

SEQ ID NO: 168 - protein, 214 aa, synthetic construct; identical to SEQ ID NO: 148
  REGION 1..214: note = Humanized clone 11 of 20D9, light chain sequence without a signalpeptide

SEQ ID NO: 169 - protein, 107 aa, synthetic construct; identical to SEQ ID NO: 149
  REGION 1..107: note = Humanized clone 11 of 20D9, light chain variable region without asignal peptide

SEQ ID NO: 170 - protein, 11 aa, synthetic construct
  REGION 1..11: note = Humanized clone 11 of 20D9, light chain variable region, CDR-L1
RASQSINKELN

SEQ ID NO: 171 - protein, 7 aa, synthetic construct
  REGION 1..7: note = Humanized clone 11 of 20D9, light chain variable region, CDR-L2
NTNNLQS

SEQ ID NO: 172 - protein, 9 aa, synthetic construct
  REGION 1..9: note = Humanized clone 11 of 20D9, light chain variable region, CDR-L3
FQHKSWPLT

SEQ ID NO: 173 - protein, 448 aa, synthetic construct; identical to SEQ ID NO: 93
  REGION 1..448: note = Humanized clone 12 of 20D9, heavy chain sequence without a signalpeptide

SEQ ID NO: 174 - protein, 118 aa, synthetic construct; identical to SEQ ID NO: 94
  REGION 1..118: note = Humanized clone 12 of 20D9, heavy chain variable region without asignal peptide

SEQ ID NO: 175 - protein, 5 aa, synthetic construct
  REGION 1..5: note = Humanized clone 12 of 20D9, heavy chain variable region, CDR-H1
NYWMT

SEQ ID NO: 176 - protein, 17 aa, synthetic construct
  REGION 1..17: note = Humanized clone 12 of 20D9, heavy chain variable region, CDR-H2
SITKTGGGTYYADSVKG

SEQ ID NO: 177 - protein, 9 aa, synthetic construct
  REGION 1..9: note = Humanized clone 12 of 20D9, heavy chain variable region, CDR-H3
LQQLGVMDA

SEQ ID NO: 178 - protein, 214 aa, synthetic construct; identical to SEQ ID NO: 148
  REGION 1..214: note = Humanized clone 12 of 20D9, light chain sequence without a signalpeptide

SEQ ID NO: 179 - protein, 107 aa, synthetic construct; identical to SEQ ID NO: 149
  REGION 1..107: note = Humanized clone 12 of 20D9, light chain variable region without asignal peptide

SEQ ID NO: 180 - protein, 11 aa, synthetic construct
  REGION 1..11: note = Humanized clone 12 of 20D9, light chain variable region, CDR-L1
RASQSINKELN

SEQ ID NO: 181 - protein, 7 aa, synthetic construct
  REGION 1..7: note = Humanized clone 12 of 20D9, light chain variable region, CDR-L2
NTNNLQS

SEQ ID NO: 182 - protein, 9 aa, synthetic construct
  REGION 1..9: note = Humanized clone 12 of 20D9, light chain variable region, CDR-L3
FQHKSWPLT

SEQ ID NO: 183 - protein, 448 aa, synthetic construct
  REGION 1..448: note = Humanized clone 13 of 20D9, heavy chain sequence without a signalpeptide
QVQLVESGGGVVQPGRSLRLSCAASGFTFNNYWMTWVRQAPGKGLEWIASITKTGGGTYYADSVKGRFTISRDNSKNTLYLQMNSLRAEDTAVYYCTTLQQLGVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 118 aa, synthetic construct; identical to SEQ ID NO: 84
  REGION 1..118: note = Humanized clone 13 of 20D9, heavy chain variable region without asignal peptide

SEQ ID NO: 185 - protein, 5 aa, synthetic construct
  REGION 1..5: note = Humanized clone 13 of 20D9, heavy chain variable region, CDR-H1
NYWMT

SEQ ID NO: 186 - protein, 17 aa, synthetic construct
  REGION 1..17: note = Humanized clone 13 of 20D9, heavy chain variable region, CDR-H2
SITKTGGGTYYADSVKG

SEQ ID NO: 187 - protein, 9 aa, synthetic construct
  REGION 1..9: note = Humanized clone 13 of 20D9, heavy chain variable region, CDR-H3
LQQLGVMDA

SEQ ID NO: 188 - protein, 214 aa, synthetic construct; identical to SEQ ID NO: 68
  REGION 1..214: note = Humanized clone 13 of 20D9, light chain sequence without a signalpeptide

SEQ ID NO: 189 - protein, 107 aa, synthetic construct; identical to SEQ ID NO: 69
  REGION 1..107: note = Humanized clone 13 of 20D9, light chain variable region without asignal peptide

SEQ ID NO: 190 - protein, 11 aa, synthetic construct
  REGION 1..11: note = Humanized clone 13 of 20D9, light chain variable region, CDR-L1
RASQNINKELN

SEQ ID NO: 191 - protein, 7 aa, synthetic construct
  REGION 1..7: note = Humanized clone 13 of 20D9, light chain variable region, CDR-L2
NTNNLQS

SEQ ID NO: 192 - protein, 9 aa, synthetic construct
  REGION 1..9: note = Humanized clone 13 of 20D9, light chain variable region, CDR-L3
FQHKSWPLT

SEQ ID NO: 193 - protein, 19 aa, synthetic construct
  REGION 1..19: note = Exemplary heavy chain signal sequence
MDWTWRILFLVAAATGAHS

SEQ ID NO: 194 - protein, 19 aa, synthetic construct
  REGION 1..19: note = Exemplary light chain signal sequence
MLPSQLIGFLLLWVPASRG

SEQ ID NO: 195 - protein, 28 aa, synthetic construct
CSSDNEYFYVDFREYEYDLKWEFPRENL

(19) United States
(12) Patent Application Publication
(10) Pub. No.: US 2025/0049939 A1
(43) Pub. Date: Feb. 13, 2025

(54) NOVEL FLT3 ANTIBODIES AND ANTIBODY-DRUG-CONJUGATES BASED THEREON, THERAPEUTIC METHODS AND USES THEREOF IN COMBINATION WITH TYROSINE KINASE INHIBITORS

(71) Applicants: TUBULIS GmbH, Planegg/Martinsried (DE); LUDWIG-MAXIMILIANS-UNIVERSITAET MUENCHEN, Munich (DE); FORSCHUNGSVERBUND BERLIN E.V., Berlin (DE); Helmholtz Zentrum Muenchen Deutsches forschungszentrum für Gesundheit und Umwelt (GmbH), Neuherberg (DE)

(72) Inventors: Heinrich LEONHARDT, Munich (DE); Andreas STENGL, Munich (DE); Maike ROAS, Munich (DE); Jonathan SCHWACH, Munich (DE); Karsten SPIEKERMANN, Munich (DE); Harald POLZER, Munich (DE); Marina ABLE, Munich (DE); Jonas HELMA-SMETS, Planegg/Martinsried (DE); Dominik SCHUMACHER, Planegg/Martinsried (DE); Marcus GERLACH, Planegg/Martinsried (DE); Christian Peter Richard HACKENBERGER, Berlin (DE); Marc-André KASPER, Berlin (DE); Irmela JEREMIAS, Neuherberg (DE); Binje VICK, Neuherberg (DE); Elisabeth KREMMER, Neuherberg (DE)

(21) Appl. No.: 18/717,535

(22) PCT Filed: Dec. 12, 2022
(86) PCT No.: PCT/EP2022/085396

§ 371 Date: Jun. 7, 2024

(30) Foreign Application Priority Data
Dec. 10, 2021 (EP) 21213755.8

(51) Int. Cl. A61K 47/68; A61P 35/02; C07K 16/28

(52) U.S. Cl. CPC A61K 47/6849; A61K 47/68031; A61P 35/02; C07K 16/2863; C07K 2317/24; C07K 2317/77; C07K 2317/92

(57) ABSTRACT

The present invention relates to novel anti-FLT3 antibodies for specifically targeting extracellular domain of FLT3. The present invention further relates to targeting FLT3 by novel antibody-drug-conjugates (ADCs) based on the novel anti-FLT3 antibodies of the present invention, especially in combination with kinase inhibitors, for use in therapy and/or for use in a method of cancer treatment (e.g., acute myeloid leukemia (AML) with or without internal tandem duplication (ITD) mutations in the FLT3 gene (FLT3-ITD)).

This application contains a Sequence Listing in computer readable form, which is incorporated herein by reference.

### TECHNICAL FIELD

The present invention relates to novel anti-FLT3 antibodies for specifically targeting extracellular domain of FLT3. The present invention further relates to targeting FLT3 by novel antibody-drug-conjugates (ADCs) based on the novel anti-FLT3 antibodies of the present invention, especially in combination with kinase inhibitors, for use in therapy and/or for use in a method of cancer treatment (e.g., acute myeloid leukemia (AML) with or without internal tandem duplication (ITD) mutations in the FLT3 gene (FLT3-ITD)).

Fms-like tyrosine kinase 3 (Receptor-type tyrosine-protein kinase or FLT3) is often constitutively activated by overexpression and ITD/TKD mutations in acute myeloid leukemia (AML). Despite the use of tyrosine kinase inhibitors in FLT3 ITD positive AML, the prognosis of patients is still poor and further improvement of therapy is required. The present invention relates to novel anti-FLT3 antibodies for specifically targeting the extracellular domain of FLT3. The present invention further relates to targeting FLT3 by novel Antibody-Drug-Conjugates (ADCs) based on the novel anti-FLT3 antibodies of the present invention, especially in combination with kinase inhibitors, for use in therapy and/or for use in a method of cancer treatment (e.g., acute myeloid leukemia (AML) with or without internal tandem duplication (ITD) mutations in the FLT3 gene (FLT3-ITD)). The ADCs of the present invention are unique therapeutic agents capable of significant tumor reduction and/or even durable complete tumor remission in AML xenograft models. Furthermore, the ADCs of the present invention when used in combination with a TKI (e.g., Midostaurin, Sorafenib, Lestaurtinib, Sunitinib, Quizartinib, Crenolanib or Gilteritinib) are capable of a strong synergy which may be used for the treatment of FLT3-ITD+ aggressive AML.

### BACKGROUND OF THE INVENTION

Acute myeloid leukemia (AML) is a malignant clonal myeloid malignancy characterized by uncontrolled growth of differentiation arrested hematopoietic stem and progenitor cells. The 5-year survival rate in the US is about 29.5% (SEER data, 2011-2017) and shows the high medical need to improve therapy. Approaches to increase the efficacy of the standard 7+3 chemotherapy include the combination with targeting agents such as the receptor tyrosine kinase (RTK) inhibitors (TKI) midostaurin and gilteritinib and the reapproved gemtuzumab-ozogamicin, an antibody drug conjugate (ADC) against CD33. ADCs are an emerging therapeutic class in the field of cancer therapy, that combines the specificity of antibodies with a highly potent drug. The well-described mechanism of action for ADC drugs includes binding of the antibody to the target in tumor cells, internalizing the ADC and releasing the cytotoxic payload to kill the target cells. Currently, several ADCs are being investigated in preclinical settings for AML treatment, targeting for example CLL-1, CD-123, IL3RA or CXCR4 and Fms-like tyrosine kinase 3 (FLT3). The latter is a member of the class III protein RTK and represents a prominent and established receptor for targeted therapys. Binding of FLT3-ligand induces phosphorylation, internalization and subsequent activation of downstream targets involved in survival and expansion of hematopoietic cells. In healthy tissue, FLT3 cell surface expression is restricted to granulocytes/macrophage progenitors, a subset of HSCs and differentiated monocytes and dendritic cells. Remarkably, it is expressed in 78% on blasts and leukemic stem cells of AML patients, the levels are significantly higher compared to healthy tissue and high levels of FLT3 were reported as risk factor for prognosis. Further, activating internal tandem duplication (ITD) mutations of FLT3 are among the most frequent genetic abnormalities in AML and occur in around 30% of patients at diagnosis. FLT3-ITD is associated with a high risk of relapse and a poor clinical outcome. Following, FLT3 targeting agents like Midostaurin are successfully applied in AML treatment. However, despite the use of tyrosine kinase inhibitors in FLT3 ITD positive AML, the prognosis of patients is still poor and further improvement of therapy is needed.

It is the object of the invention to comply with the needs in the prior art. The present invention complies with the needs inter alia by providing novel FLT3-targeting antibody-drug-conjugates (ADCs), which were generated applying the known P5 conjugation technology (e.g., WO2018/041985), which is based on the modification of interchain-Cysteine residues with unsaturated phosphonamidate reagents.

### SUMMARY OF THE INVENTION

The present invention relates to an anti-FLT3 antibody (Receptor-type tyrosine-protein kinase FLT3), wherein said anti-FLT3 antibody is capable of the following: (a) binding to an extracellular domain of said FLT3; (b) cross-reactivity with cynomolgus monkey (e.g., Macaca fascicularis) FLT3 (e.g., having UniProtKB Accession Number: Q95M30); (c) internalizing, preferably antigen-mediated antibody internalization; (d) binding to an extracellular epitope of said FLT3, wherein said extracellular epitope comprising one or more of the following sequences: SEQ ID NO: 2 (KSSSYPM); SEQ ID NO: 3 (SQGESCK); and/or SEQ ID NO: 4 (DGYP); (e) preferably said FLT3 is a human FLT3 having SEQ ID NO: 1 or UniProtKB Accession Number: P36888, further preferably said extracellular domain ranges from amino acids 27 to 543 of SEQ ID NO: 1.

The present invention further relates to an antibody drug conjugate (ADC) comprising the anti-FLT3 antibody of the present invention conjugated via its interchain disulfidebond forming cysteine residues to a phosphonamidate linked, cathepsin B cleavable monomethyl auristatin F (MMAF) cytotoxic drug having Formula I (i.e., a structure of the linker-payload before conjugation):

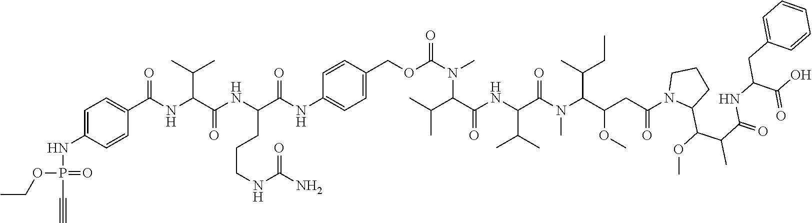 [SMILES: C#CP(=O)(Nc1ccc(C(=O)NC(C(=O)NC(CCCNC(N)=O)C(=O)Nc2ccc(COC(=O)N(C)C(C(=O)NC(C(=O)N(C)C(C(C)CC)C(CC(=O)N3CCCC3C(OC)C(C)C(=O)NC(Cc3ccccc3)C(=O)O)OC)C(C)C)C(C)C)cc2)C(C)C)cc1)OCC]

which, after the conjugation with an antibody of the present invention, may have Formula II:

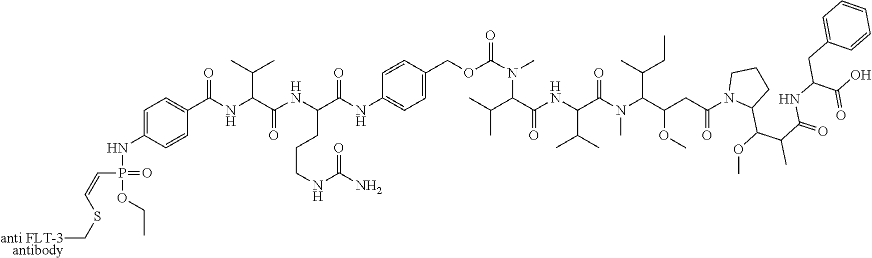

Preferably, the antibody might be conjugated to 0-8 drug molecules.

Particularly, in vitro, the ADCs of the present invention are capable of mediating potent cytotoxicity to FLT3 wt and FLT3-ITD expressing Ba/F3 cell lines, AML cell lines and patient derived xenograft AML cells. Furthermore, in vivo, the ADC treatment with the present invention is capable of leading to significant tumor reduction or even durable complete remission in AML xenograft models. Importantly, the ADCs of the present invention demonstrate no severe hematotoxicity in in vitro colony formation assays using concentrations which are cytotoxic in AML cell line treatment. Combination of the ADC of the present invention with Midostaurin, a receptor tyrosine kinase inhibitor (TKI), is capable of strong synergy in vitro and in vivo leading to curing or long term remission or durable reduction (e.g., below detection limit) of FLT3-ITD+ aggressive AML cell models. This indicates that targeting of FLT3 with an advanced new generation ADC of the present invention is a promising and potent antileukemic strategy, especially when combined with FLT3-TKI in FLT3-ITD+ AML.

The present invention further relates to a composition or kit comprising one or more anti-FLT3 antibodies of the present invention and/or one or more antibody drug conjugates (ADCs) of the present invention. Particularly, the present invention further relates to the composition or kit of the present invention, wherein said composition or kit is a pharmaceutical and/or diagnostic composition or kit, wherein said pharmaceutical or diagnostic composition further comprising a suitable receptor tyrosine kinase inhibitor (TKI), preferably said TKI is selected from the group consisting of: Midostaurin (PKC 412), Sorafenib, Lestaurtinib, Sunitinib, Quizartinib, Crenolanib, Gilteritinib. The present invention further relates to a method of/for treatment, amelioration, prophylaxis and/or diagnostics of cancer, preferably said cancer is acute myeloid leukemia (AML), further preferably said AML comprising internal tandem duplication (ITD) mutations in FLT3 (FLT3-ITD), said method comprising: administering a therapeutically or prophylactically effective amount of the antibody, antibody drug conjugate (ADC), composition and/or kit of the present invention to a subject in need thereof.

Based on the above, the present application satisfies this demand/need of the prior art by providing such novel FLT3 antibodies and ADCs based thereon as well as methods and uses thereof described herein below, characterized in the claims and illustrated by the appended Examples and Figures.

### Overview of the Sequence Listing

As described herein and unless otherwise stated references are made to UniProtKB Accession Numbers (https://www.uniprot.org/news/2021/06/02/release, e.g., as available in UniProt release 2021_03, Published Jun. 2, 2021).

As described herein and unless otherwise stated references are made to NCBI GenBank Accession Numbers (https://www.ncbi.nlm.nih.gov/genbank/release/current/, e.g., as available in NCBI-GenBank Release 245.0, Aug. 15, 2021).

SEQ ID NO: 1 is the protein sequence of the human receptor-type tyrosine-protein kinase FLT3, UniprotKB Accession Number: P36888-1.

SEQ ID NO: 2 is an exemplary epitope 1 polypeptide sequence of the present invention, to which an antibody of the present invention may bind.

SEQ ID NO: 3 is an exemplary epitope 2 polypeptide sequence of the present invention, to which an antibody of the present invention may bind.

SEQ ID NO: 4 is an exemplary epitope 3 polypeptide sequence of the present invention, to which an antibody of the present invention may bind.

SEQ ID NO: 5 is a V_{H} protein sequence of the 20D9 Ab clone without a signal sequence.

SEQ ID NO: 6 is a V_{L} protein sequence of the 20D9 Ab clone without a signal sequence.

SEQ ID NO: 7 is a V_{H} protein sequence of the 2F12 Ab clone without a signal sequence.

SEQ ID NO: 8 is a V_{L} protein sequence of the 2F12 Ab clone without a signal sequence.

SEQ ID NO: 9 is a V_{H} protein sequence of the 4B12 Ab clone without a signal sequence.

SEQ ID NO: 10 is a V_{L} protein sequence of the 4B12 Ab clone without a signal sequence.

SEQ ID NO: 11 is a V_{H} protein sequence of the 27E7 Ab clone without a signal sequence.

SEQ ID NO: 12 is a V_{L} protein sequence of the 27E7 Ab clone without a signal sequence.

SEQ ID NO: 13 is a V_{H} protein sequence of the 29H1 Ab clone without a signal sequence.

SEQ ID NO: 14 is a V_{L} protein sequence of the 29H1 Ab clone without a signal sequence.

SEQ ID NO: 15 is a V_{H} protein sequence of the 30B12 Ab clone without a signal sequence.

SEQ ID NO: 16 is a V_{L} protein sequence of the 30B12 Ab clone without a signal sequence.

SEQ ID NO: 17 is a V_{H} protein sequence of the 19H5 Ab clone without a signal sequence.

SEQ ID NO: 18 is a V_{L} protein sequence of the 19H5 Ab clone without a signal sequence.

SEQ ID NO: 19 is a signal sequence of the variable region of the heavy chain.

SEQ ID NO: 20 is a signal sequence of the variable region of the light chain.

SEQ ID NOS: 21-62 are examplary polypeptide sequences of the CDR regions (e.g., CDR-H1, CDR-H2, CDR-H3 corresponding CDR-L1, CDR-L2 and CDR-L3 regions) of the exemplary antibodies of the present invention.

SEQ ID NO: 63 humanized clone 1 of 20D9, heavy chain sequence without a signal peptide.

SEQ ID NO: 64 humanized clone 1 of 20D9, heavy chain variable region without a signal peptide.

SEQ ID NO: 65 humanized clone 1 of 20D9, heavy chain variable region, CDR-H1.

SEQ ID NO: 66 humanized clone 1 of 20D9, heavy chain variable region, CDR-H2.

SEQ ID NO: 67 humanized clone 1 of 20D9, heavy chain variable region, CDR-H3.

SEQ ID NO: 68 humanized clone 1 of 20D9, light chain sequence without a signal peptide.

SEQ ID NO: 69 humanized clone 1 of 20D9, light chain variable region without a signal peptide.

SEQ ID NO: 70 humanized clone 1 of 20D9, light chain variable region, CDR-L1.

SEQ ID NO: 71 humanized clone 1 of 20D9, light chain variable region, CDR-L2.

SEQ ID NO: 72 humanized clone 1 of 20D9, light chain variable region, CDR-L3.

SEQ ID NO: 73 humanized clone 2 of 20D9, heavy chain sequence without a signal peptide.

SEQ ID NO: 74 humanized clone 2 of 20D9, heavy chain variable region without a signal peptide.

SEQ ID NO: 75 humanized clone 2 of 20D9, heavy chain variable region, CDR-H1.

SEQ ID NO: 76 humanized clone 2 of 20D9, heavy chain variable region, CDR-H2.

SEQ ID NO: 77 humanized clone 2 of 20D9, heavy chain variable region, CDR-H3.

SEQ ID NO: 78 humanized clone 2 of 20D9, light chain sequence without a signal peptide.

SEQ ID NO: 79 humanized clone 2 of 20D9, light chain variable region without a signal peptide.

SEQ ID NO: 80 humanized clone 2 of 20D9, light chain variable region, CDR-L1.

SEQ ID NO: 81: Humanized clone 2 of 20D9, light chain variable region, CDR-L2.

SEQ ID NO: 82: Humanized clone 2 of 20D9, light chain variable region, CDR-L3.

SEQ ID NO: 83: Humanized clone 3 of 20D9, heavy chain sequence without a signal peptide.

SEQ ID NO: 84: Humanized clone 3 of 20D9, heavy chain variable region without a signal peptide.

SEQ ID NO: 85: Humanized clone 3 of 20D9, heavy chain variable region, CDR-H1.

SEQ ID NO: 86: Humanized clone 3 of 20D9, heavy chain variable region, CDR-H2.

SEQ ID NO: 87: Humanized clone 3 of 20D9, heavy chain variable region, CDR-H3.

SEQ ID NO: 88: Humanized clone 3 of 20D9, light chain sequence without a signal peptide.

SEQ ID NO: 89: Humanized clone 3 of 20D9, light chain variable region without a signal peptide.

SEQ ID NO: 90: Humanized clone 3 of 20D9, light chain variable region, CDR-L1.

SEQ ID NO: 91: Humanized clone 3 of 20D9, light chain variable region, CDR-L2.

SEQ ID NO: 92: Humanized clone 3 of 20D9, light chain variable region, CDR-L3.

SEQ ID NO: 93: Humanized clone 4 of 20D9, heavy chain sequence without a signal peptide.

SEQ ID NO: 94: Humanized clone 4 of 20D9, heavy chain variable region without a signal peptide.

SEQ ID NO: 95: Humanized clone 4 of 20D9, heavy chain variable region, CDR-H1.

SEQ ID NO: 96: Humanized clone 4 of 20D9, heavy chain variable region, CDR-H2.

SEQ ID NO: 97: Humanized clone 4 of 20D9, heavy chain variable region, CDR-H3.

SEQ ID NO: 98: Humanized clone 4 of 20D9, light chain sequence without a signal peptide.

SEQ ID NO: 99: Humanized clone 4 of 20D9, light chain variable region without a signal peptide.

SEQ ID NO: 100: Humanized clone 4 of 20D9, light chain variable region, CDR-L1.

SEQ ID NO: 101: Humanized clone 4 of 20D9, light chain variable region, CDR-L2.

SEQ ID NO: 102: Humanized clone 4 of 20D9, light chain variable region, CDR-L3.

SEQ ID NO: 103: Humanized clone 5 of 20D9, heavy chain sequence without a signal peptide.

SEQ ID NO: 104: Humanized clone 5 of 20D9, heavy chain variable region without a signal peptide.

SEQ ID NO: 105: Humanized clone 5 of 20D9, heavy chain variable region, CDR-H1.

SEQ ID NO: 106: Humanized clone 5 of 20D9, heavy chain variable region, CDR-H2.

SEQ ID NO: 107: Humanized clone 5 of 20D9, heavy chain variable region, CDR-H3.

SEQ ID NO: 108: Humanized clone 5 of 20D9, light chain sequence without a signal peptide.

SEQ ID NO: 109: Humanized clone 5 of 20D9, light chain variable region without a signal peptide.

SEQ ID NO: 110: Humanized clone 5 of 20D9, light chain variable region, CDR-L1.

SEQ ID NO: 111: Humanized clone 5 of 20D9, light chain variable region, CDR-L2.

SEQ ID NO: 112: Humanized clone 5 of 20D9, light chain variable region, CDR-L3.

SEQ ID NO: 113: Humanized clone 6 of 20D9, heavy chain sequence without a signal peptide.

SEQ ID NO: 114: Humanized clone 6 of 20D9, heavy chain variable region without a signal peptide.

SEQ ID NO: 115: Humanized clone 6 of 20D9, heavy chain variable region, CDR-H1.

SEQ ID NO: 116: Humanized clone 6 of 20D9, heavy chain variable region, CDR-H2.

SEQ ID NO: 117: Humanized clone 6 of 20D9, heavy chain variable region, CDR-H3.

SEQ ID NO: 118: Humanized clone 6 of 20D9, light chain sequence without a signal peptide.

SEQ ID NO: 119: Humanized clone 6 of 20D9, light chain variable region without a signal peptide.

SEQ ID NO: 120: Humanized clone 6 of 20D9, light chain variable region, CDR-L1.

SEQ ID NO: 121: Humanized clone 6 of 20D9, light chain variable region, CDR-L2.

SEQ ID NO: 122: Humanized clone 6 of 20D9, light chain variable region, CDR-L3.

SEQ ID NO: 123: Humanized clone 7 of 20D9, heavy chain sequence without a signal peptide.

SEQ ID NO: 124: Humanized clone 7 of 20D9, heavy chain variable region without a signal peptide.

SEQ ID NO: 125: Humanized clone 7 of 20D9, heavy chain variable region, CDR-H1.

SEQ ID NO: 126: Humanized clone 7 of 20D9, heavy chain variable region, CDR-H2.

SEQ ID NO: 127: Humanized clone 7 of 20D9, heavy chain variable region, CDR-H3.

SEQ ID NO: 128: Humanized clone 7 of 20D9, light chain sequence without a

signal peptide.

SEQ ID NO: 129: Humanized clone 7 of 20D9, light chain variable region without a signal peptide.

SEQ ID NO: 130: Humanized clone 7 of 20D9, light chain variable region, CDR-L1.

SEQ ID NO: 131: Humanized clone 7 of 20D9, light chain variable region, CDR-L2.

SEQ ID NO: 132: Humanized clone 7 of 20D9, light chain variable region, CDR-L3.

SEQ ID NO: 133: Humanized clone 8 of 20D9, heavy chain sequence without a signal peptide.

SEQ ID NO: 134: Humanized clone 8 of 20D9, heavy chain variable region without a signal peptide.

SEQ ID NO: 135: Humanized clone 8 of 20D9, heavy chain variable region, CDR-H1.

SEQ ID NO: 136: Humanized clone 8 of 20D9, heavy chain variable region, CDR-H2.

SEQ ID NO: 137: Humanized clone 8 of 20D9, heavy chain variable region, CDR-H3.

SEQ ID NO: 138: Humanized clone 8 of 20D9, light chain sequence without a signal peptide.

SEQ ID NO: 139: Humanized clone 8 of 20D9, light chain variable region without a signal peptide.

SEQ ID NO: 140: Humanized clone 8 of 20D9, light chain variable region, CDR-L1.

SEQ ID NO: 141: Humanized clone 8 of 20D9, light chain variable region, CDR-L2.

SEQ ID NO: 142: Humanized clone 8 of 20D9, light chain variable region, CDR-L3.

SEQ ID NO: 143: Humanized clone 9 of 20D9, heavy chain sequence without a signal peptide.

SEQ ID NO: 144: Humanized clone 9 of 20D9, heavy chain variable region without a signal peptide.

SEQ ID NO: 145: Humanized clone 9 of 20D9, heavy chain variable region, CDR-H1.

SEQ ID NO: 146: Humanized clone 9 of 20D9, heavy chain variable region, CDR-H2.

SEQ ID NO: 147: Humanized clone 9 of 20D9, heavy chain variable region, CDR-H3.

SEQ ID NO: 148: Humanized clone 9 of 20D9, light chain sequence without a signal peptide.

SEQ ID NO: 149: Humanized clone 9 of 20D9, light chain variable region without a signal peptide.

SEQ ID NO: 150: Humanized clone 9 of 20D9, light chain variable region, CDR-L1.

SEQ ID NO: 151: Humanized clone 9 of 20D9, light chain variable region, CDR-L2.

SEQ ID NO: 152: Humanized clone 9 of 20D9, light chain variable region, CDR-L3.

SEQ ID NO: 153: Humanized clone 10 of 20D9, heavy chain sequence without a signal peptide.

SEQ ID NO: 154: Humanized clone 10 of 20D9, heavy chain variable region without a signal peptide.

SEQ ID NO: 155: Humanized clone 10 of 20D9, heavy chain variable region, CDR-H1.

SEQ ID NO: 156: Humanized clone 10 of 20D9, heavy chain variable region, CDR-H2.

SEQ ID NO: 157: Humanized clone 10 of 20D9, heavy chain variable region, CDR-H3.

SEQ ID NO: 158: Humanized clone 10 of 20D9, light chain sequence without a signal peptide.

SEQ ID NO: 159: Humanized clone 10 of 20D9, light chain variable region without a signal peptide.

SEQ ID NO: 160: Humanized clone 10 of 20D9, light chain variable region, CDR-L1.

SEQ ID NO: 161: Humanized clone 10 of 20D9, light chain variable region, CDR-L2.

SEQ ID NO: 162: Humanized clone 10 of 20D9, light chain variable region, CDR-L3.

SEQ ID NO: 163: Humanized clone 11 of 20D9, heavy chain sequence without a signal peptide.

SEQ ID NO: 164: Humanized clone 11 of 20D9, heavy chain variable region without a signal peptide.

SEQ ID NO: 165: Humanized clone 11 of 20D9, heavy chain variable region, CDR-H1.

SEQ ID NO: 166: Humanized clone 11 of 20D9, heavy chain variable region, CDR-H2.

SEQ ID NO: 167: Humanized clone 11 of 20D9, heavy chain variable region, CDR-H3.

SEQ ID NO: 168: Humanized clone 11 of 20D9, light chain sequence without a signal peptide.

SEQ ID NO: 169: Humanized clone 11 of 20D9, light chain variable region without a signal peptide.

SEQ ID NO: 170: Humanized clone 11 of 20D9, light chain variable region, CDR-L1.

SEQ ID NO: 171: Humanized clone 11 of 20D9, light chain variable region, CDR-L2.

SEQ ID NO: 172: Humanized clone 11 of 20D9, light chain variable region, CDR-L3.

SEQ ID NO: 173: Humanized clone 12 of 20D9, heavy chain sequence without a signal peptide.

SEQ ID NO: 174: Humanized clone 12 of 20D9, heavy chain variable region without a signal peptide.

SEQ ID NO: 175: Humanized clone 12 of 20D9, heavy chain variable region, CDR-H1.

SEQ ID NO: 176: Humanized clone 12 of 20D9, heavy chain variable region, CDR-H2.

SEQ ID NO: 177: Humanized clone 12 of 20D9, heavy chain variable region, CDR-H3.

SEQ ID NO: 178: Humanized clone 12 of 20D9, light chain sequence without a signal peptide.

SEQ ID NO: 179: Humanized clone 12 of 20D9, light chain variable region without a signal peptide.

SEQ ID NO: 180: Humanized clone 12 of 20D9, light chain variable region, CDR-L1.

SEQ ID NO: 181: Humanized clone 12 of 20D9, light chain variable region, CDR-L2.

SEQ ID NO: 182: Humanized clone 12 of 20D9, light chain variable region, CDR-L3.

SEQ ID NO: 183: Humanized clone 13 of 20D9, heavy chain sequence without a signal peptide.

SEQ ID NO: 184: Humanized clone 13 of 20D9, heavy chain variable region without a signal peptide.

SEQ ID NO: 185: Humanized clone 13 of 20D9, heavy chain variable region, CDR-H1.

SEQ ID NO: 186: Humanized clone 13 of 20D9, heavy chain variable region, CDR-H2.

SEQ ID NO: 187: Humanized clone 13 of 20D9, heavy chain variable region, CDR-H3.

SEQ ID NO: 188: Humanized clone 13 of 20D9, light chain sequence without a signal peptide.

SEQ ID NO: 189: Humanized clone 13 of 20D9, light chain variable region without a signal peptide.

SEQ ID NO: 190: Humanized clone 13 of 20D9, light chain variable region, CDR-L1.

SEQ ID NO: 191: Humanized clone 13 of 20D9, light chain variable region, CDR-L2.

SEQ ID NO: 192: Humanized clone 13 of 20D9, light chain variable region, CDR-L3.

SEQ ID NO: 193: exemplary heavy chain signal sequence of the present invention.

SEQ ID NO: 194: exemplary light chain signal sequence of the present invention.

SEQ ID NO: 195: amino acid duplication sequence of FLT3-NPOS mutant.

### BRIEF DESCRIPTION OF THE FIGURES

FIG. 1: Evaluation of epitope specificity and internalization capability of anti-FLT3 monoclonal antibodies. (A) Affinity of seven anti-FLT3 monoclonal antibody (mab) clones to recombinant human FLT3 measured in enzyme-linked immunosorbent assay (ELISA) normalized to the binding of the antibodies to BSA control. Dissociation constant (K_{D}; mean±s.d. . . . ; n=3) is depicted. (B) Schematic FLT3 receptor. Black arrows indicate common mutations and blue arrows indicate the three identified epitopes of seven anti-FLT3 antibodies analyzed in linear epitope mapping by PEPperPRINT. Figure was created with BioRender.com. (C) Binding and temperature induced internalization of anti-FLT3 antibodies in Ba/F3 cells expressing human wildtype FLT3. Internalization was induced by incubation for 30 min at 37° C. (grey) compared to 4° C. (black). Remaining surface-bound antibody was detected in flow cytometry. MFI was normalized to control human IgG1 binding. MFI=mean fluorescence intensity. Unpaired, two-tailed Student's t-test; *p<0.05; mean±s.d. of n=3 is depicted. (D) Cell surface binding of 20D9 mab or control human IgG1 antibody to Ba/F3 cells stably expressing pMIY (Ba/F3-pMIY) empty vector (ev), human wildtype (hFLT3 wt) or ITD mutant human FLT3 (hFLT3ITD) was measured in flow cytometry. Mean±s.d. of n=3. (E) Temperature induced internalization of 4B12 mab in MOLM-13 (FLT3 positive) cells after 30 min incubation at 37° C. compared to 4° C. and HL-60 (FLT3 negative) after 37° C. incubation assessed in immunofluorescence staining. Red=FLT3 staining by 4B12, Green=membrane staining by Vybrant DiO, Blue=nuclear staining by DAPI. Scale bar 5 μm. Representative pictures are shown. (F) Cell surface binding of 20D9 mab or control hIgG1 antibody to Ba/F3-pMIY ev, hFLT3 wt, murine (mFLT3 wt) or cynomolgues monkey FLT3 (cynoFLT3 wt) was measured in flow cytometry. Mean±s.d. of n=3. (G) Cell surface binding of 20D9 mab or control hIgG1 antibody to Ba/F3-PMIY ev, hFLT3 wt or epitope mutant FLT3 (hFLT3/S50P/P54R) measured in flow cytometry. Mean±s.d. of n=3. (H) Cell surface binding of 20D9 mab or control hIgG1 antibody to Ba/F3-PMIY ev, hFLT3 wt or human CD64 (hCD64) measured in flow cytometry. Mean±s.d. of n=3.

FIG. 2: Analysis of cytotoxicity of FLT3-specific 20D9-ADC to different FLT3 variants. (A) Schematic process of P5 conjugation technology via disulfite bond reduction and Staudinger induced Michael addition. Final ADC consists of monoclonal antibody (20D9 or IgG1 antibody) coupled to monomethyl-auristatin F toxin. Figure was created with BioRender.com. (B-E,G) Assessment of cytotoxicity of ADCs in different Ba/F3 cell lines. Viability was determined after 72 h treatment with different concentrations by trypan blue exclusion count and compared to untreated control. Mean±s.d. of n=3 biological replicates. (B) Treatment of Ba/F3-pMIY ev, hFLT3 wt and hFLT3ITD with 20D9-ADC. (C) Treatment of Ba/F3-PMIY ev, hFLT3 wt, mFLT3 wt and cynoFLT3 wt with 20D9-ADC. (D) Treatment of Ba/F3-PMIY ev, hFLT3 wt and hFLT3/S50P/P54R with 20D9-ADC. (E) Treatment of Ba/F3-PMIY ev or human FCGR1 (CD64) with either 20D9-ADC or IgG1-ADC. (F) IC50 values of 20D9-ADC and IgG1-ADC in Ba/F3-CD64 and Ba/F3-CD64-FLT3 cell lines. Calculation based on data from FIGS. 2E and 2G calculated by GraphPad Prism. Unpaired, two-tailed Student's t-test; *p<0.05. Each dot represents a replicate, horizontal line indicates mean. (G) Treatment of Ba/F3-PMIY ev or human FCGR1 and human FLT3 (CD64-FLT3) with different concentrations of either 20D9-ADC or IgG1-ADC.

FIG. 3: Analysis of 20D9-ADC and control IgG1-ADC cytotoxicity in leukemia and lymphoma cell lines. (A) Correlation of MFI of CD64 cell surface expression and MFI of FLT3 cell surface expression of myeloid human cell lines measure in flow cytometry. Expression data presented in Supplementary FIGS. 4A and B. Black line indicates simple linear regression with error interval. r=Pearson correlation coefficient; r²=Coefficient of determination; p=p value from two tailed test with confidence interval of 95%. MFI=mean fluorescence intensity. (B,C,E,F) Assessment of cytotoxicity of ADCs in different human cell lines. Viability was determined after 96h treatment with different concentrations of ADCs by resazurin fluorescence and normalized to untreated control. Dashed line indicates 100 ng/ml drug concentration. Mean±s.d. of n=3 biological replicates. (B) Treatment of FLT3 positive human cell lines with 20D9-ADC. (C) Treatment of FLT-3 negative human cell lines with 20D9-ADC. (D) Correlation of IC50 values of 20D9-ADC and sum of MFI of FLT3 and CD64 cell surface expression of myeloid human cell lines measure in flow cytometry. Expression data presented in Supplementary FIGS. 4 A,B and IC50 values of 20D9-ADC in Supplementary Table 1. Black line indicates simple linear regression with error interval. (E) Treatment of FLT3 positive human cell lines with IgG1-ADC. (F) Treatment of MOLM-13 cells with 20D9-ADC or IgG1-ADC either native, buffer-incubated control (buffer control), or deglycosylated (deglyc.).

FIG. 4: Evaluation of in vivo activity of 20D9-ADC in xenograft mouse models. NSG mice were injected intravenously (i.v.) with 1×10⁵ luciferase expressing MOLM-13 cells (A-C) or 2×10⁶ luciferase expressing AML-573 PDX cells (D,E). Leukemic burden was monitored once or twice a week by bioluminescence imaging (BLI), and total flux was quantified. Mean±standard deviation is depicted. Treatment is indicated with rectangles in dark blue (20D9-ADC, 3 mg/kg), light blue (20D9-ADC, 1 mg/kg), grey (PBS) or black (all groups as indicated). (A) One week after transplantation, mice were treated with 20D9-ADC (1 mg/kg or 3 mg/kg, i.v.) or PBS as control (n=4/group) once a week for six weeks (1 mg/kg) or for four weeks (3 mg/kg). (B) BLI pictures of one representative mouse per group are shown. (C) One week after transplantation, mice were treated with either native or glycosylated 20D9-ADC or with either native or glycosylated IgG1-ADC (3 mg/kg; n=3/group) once a week for two weeks. PBS control mice of experiment shown in A are included as control. (D) 20 days after transplantation, mice were treated at intermediate tumor burden with 20D9-ADC (3 mg/kg) or PBS as control (n=3/group) once a week for up to four weeks (2 mice) or five weeks (one mouse). (E) PBS-treated control mice from (D) (n=3) were treated at day 41 after transplantation at advanced tumor burden with 20D9-ADC once a week for four weeks (two doses of 3 mg/kg, followed by two doses of 1 mg/kg).

FIG. 5: Analysis of Hematotoxicity of 20D9-ADC. (A) Blood analysis of NSG mice during treatment with 20D9-ADC. NSG mice were injected i.v. with AML-640 cells and treated with 20D9-ADC (3 mg/kg) or PBS once a week for 3 weeks. 6 days after the first dose (d27) or 13 days after the last dose (d48), 100 μl of peripheral blood was collected. Blood cell counts (thrombocytes, leukocytes, and hemoglobin levels) were compared to NSG mice without cell transplantation or treatment. 2way ANOVA; *p<0.05; Mean±s.d. of n=3. (B) Expression of FLT3 and CD64 in CD34 positive healthy bone marrow (BM) cells measured in flow cytometry. Mean±s.d. of n=3 donors. (B,C) CD34 positive cells were treated with 0.04 μg/ml, 0.2 μg/ml or 1 μg/ml 20D9-ADC, 1 g/ml IgG1-ADC or PBS and analyzed in flow cytometry after 4 days. Kruskal-Wallis test; *p<0.05; **p<0.01; ***p<0.001; Mean±s.d. of n=5. (C) Percentage of living cells measured with Annexin V/PI staining and normalized to PBS. (D) Differentiation assessment after staining to differentiation markers. CMP: common myeloid GMP: progenitors; granulocyte-monocyte progenitors; MEP: megakaryocyte/erythroid progenitors; MLP: multilymphoid progenitors; MPP: multipotent progenitors; hematopoietic stem cells (HSC). (E) Assessment of clonogenic capacity of healthy CD34+ BM cells. Cells were treated with 0.04 μg/ml, 0.2 μg/ml or 1 μg/ml 20D9-ADC, 1 μg/ml IgG1-ADC or PBS and plated for colony forming unit (CFU) assay. After 14 days, colonies were counted. GEMM: granulocyte, erythrocyte, macrophage, megakaryocyte. GM: granulocyte, macrophage. M: macrophage. G: granulocyte. E: erythrocyte. BFU-E: burst-forming unit erythrocyte. CFU: colony forming unit. 2way ANOVA; *p<0.05; Mean±s.d. of n=5.

FIG. 6: Treatment combination of 20D9-ADC and tyrosine kinase inhibitors. (A,B) Upregulation of FLT3 cell surface expression in MOLM-13 cells after treatment with kinase inhibitors compared to untreated control. Cells were analyzed in flow cytometry after treatment. Dotted line represents MFI ratio of untreated cells. Mean±s.d. of n=2 is depicted. (A) Cells were treated with 5, 25 or 50 nM midostaurin, quizartinib and Sorafenib for 6, 24, 48 or 72 h. (B) Cells were treated with 5 nM midostaurin, 1 nM quizartinib, 5 nM Sorafenib or 1 UM dasatinib for 72 h. (C-F) Treatment combination of 20D9-ADC and midostaurin (C,D) or quizartinib (E,F) in MOLM-13 cells compared to treatment with 20D9-ADC, midostaurin or quizartinib as single agent. Viability was determined after 96h by resazurin fluorescence and normalized to dimethyl sulfoxide (DMSO) treated control. (C,E) Each dot represents one biological replicate, the horizontal line indicates the mean. two-way ANOVA; *p<0.05; **p<0.01; ***p<0.001. Combination indices (CIs) with standard deviation were determined using CompuSyn software; CI<1 indicates synergy and is underlined; CI=1 additivity; CI>1 antagonism. (D,F) The synergy score was calculated by ‘Synergy Finder’ software using zero interaction potency (ZIP) modelling. Grey triangles indicate increasing drug concentrations. A positive Synergy score value δ and the red colouring indicate synergism. (G) Treatment combination of 20D9-ADC and midostaurin in vivo. NSG mice were injected i.v. with 1e5 luciferase expressing MOLM-13 cells. Leukemic burden was monitored once or twice a week by BLI, and total flux was quantified. Mean±standard deviation is depicted. One week after transplantation, mice were treated for three weeks with 20D9-ADC (1 mg/kg i.v., once per week), midostaurin (50 mg/kg p.o. 5 days a week), a combination of both or PBS as control (n=4/group). Bioluminescence imaging of one representative mouse of each group at day 6 and 16.

FIG. 7: Sequence analysis of the light and heavy chains of the Ab clones of the present intervention. (A) Protein sequence alignment of 2F12, 4B12, 20D9, 27E7, 29H1 and 30B12 antibody variable heavy chains. (B) Protein sequence alignment of 2F12, 4B12, 20D9, 27E7, 29H1 and 30B12 antibody variable light chains. (C-F) Protein sequence alignment of 30B12, 2F12, 4B12, 20D9, 27E7, 29H1 and 19H5 antibody variable light (C and E) or variable heavy (D and F) chain. CDR1, 2 and 3 according to the Kabat numbering system (C and D) and the Chothia numbering system (E and F) are marked with red rectangles.

FIG. 8: MS spectrum of the 20D9ADC after deglycosylation and reduction. LC/ESI-MS, DAR: 7.8, *Deconvolution artefact from HC+3MMAF, ^{#}PNGase-F.

FIG. 9: A-HIC analysis of the 20D9ADC. A-HIC chromatogram of the purified 20D9-ADC, absorbance at 220 nm, DAR7.7.

FIG. 10: SEC-HPLC analysis of the 20D9ADC. A-SEC chromatogram of the purified 20D9-ADC, absorbance at 220 nm-less than 1% aggregates.

FIG. 11: Evaluation of binding and cytotoxicity of 20D9-ADC in FLT3ITD and TKD mutants. (A,B) Cell surface expression of FLT3 receptor (A) or cell surface binding of 20D9 mab and control hIgG1 antibody (B) in Ba/F3 cells stably expressing pMIY (Ba/F3-pMIY) empty vector (ev), wildtype (hFLT3 wt) or FLT3 mutants FLT3/NPOS, FLT3/D835Y, FLT3/D835V, FLT3/NPOS D835Y, FLT3/NPOS N676K, FLT3/NPOS F6911 or FLT3/NPOS F691L was measured in flow cytometry. Mean±s.d. of n=3. (C,D) Treatment of Ba/F3 cells stably expressing pMIY (Ba/F3-PMIY) ev, FLT3 wt, FLT3 mutants FLT3/NPOS, FLT3/D835Y, FLT3/D835V (C) or FLT3/NPOS D835Y, FLT3/NPOS N676K, FLT3/NPOS F6911 FLT3/NPOS F691L (D) with different concentrations of 20D9-ADC. Viability was determined after 72h by trypan blue exclusion count and compared to untreated control. Mean±s.d. of n=3 biological replicates.

FIG. 12: Cytotoxicity of 20D9-ADC on p53 wt and KD cell lines. Cytotoxicity of 20D9-ADC in native or p53 KD MV4-11, MOLM-13 or OCI-AML 3 cells. Viability was determined after 96h treatment with different concentrations by resazurin fluorescence and normalized to untreated control. Mean±s.d. of n=3 biological replicates.

FIG. 13: Binding of humanized antibody clones 1-12 compared to the parental 20D9 to human FLT3. Binding was assessed by ELISA assay with recombinant human FLT3 coated to ELISA plates. The calculation of the dissociation constant K_{d} was performed using GraphPad Prism. Mean±s.d. of n=3 biological replicates.

FIG. 14: Binding of humanized antibody clones 1-12 compared to the parental 20D9 to human FLT3. Cell surface binding of humanized mabs to Ba/F3 cells stably expressing pMIY (Ba/F3-pMIY) empty vector (ev), human wildtype (hFLT3 wt) or cynomolgous FLT3 (cynoFLT3) was measured in flow cytometry and normalized to binding of control IgG1 antibody. Mean±s.d. of n=3.

FIG. 15: Comparison of #3-ADC and 20D9-ADC cytotoxicity. Assessment of cytotoxicity of ADCs in BaF3 cell model expressing empty vector (ev) or human FLT3 (hFLT3) and different cell lines. Viability was determined after 96h treatment with different concentrations of ADCs by resazurin fluorescence and normalized to untreated control. Mean±s.d. of n=3 biological replicates.

FIG. 16: Analysis of the effect of LALA mutation on binding of Fcγ receptors. Cell surface binding of #3 and #3-LALA and control IgG1 and IgG1-LALA mab to Ba/F3 cells stably expressing pMIY (Ba/F3-pMIY) empty vector (ev), human wildtype (hFLT3 wt) or Fcγ receptors (CD16, CD32, CD64) was measured in flow cytometry. Mean±s.d. of n=3.

FIG. 17: Analysis of #3 mab binding to FLT3 orthologues and homologues. Cell surface binding of #3 and #3-LALA mab to Ba/F3 cells stably expressing pMIY (Ba/F3-pMIY) empty vector (ev), human wildtype (hFLT3 wt) and FLT3 orthologues from mouse (mFLT3), cynomolgous monkey (cynoFLT3) and rat (ratFLT3) (A) or FLT3 homologues (VEGFR, PDGFRα, CSF-1R, c-KIT, (B)) was measured in flow cytometry. Mean±s.d. of n=3.

FIG. 18: Overview of the performance of the humanized antibody clones 1-12 in different categories and lead candidate selection (“*”-degree of sequence similarity, performance per category is indicated in dark grey=above average performance, light grey=average performance, white=below average performance).

### DETAILED DESCRIPTION OF THE INVENTION

The present inventors produced and characterized novel specific anti-FLT3 antibodies for specifically targeting the extracellular domain of FLT3. This is particularly advantageous as it relates to a new therapeutic method for treating cancer (e.g., AML). The present invention further provides targeting FLT3 by novel antibody-drug-conjugates (ADCs) based on the novel anti-FLT3 antibodies of the present invention, especially in combination with kinase inhibitors, for use in therapy and/or for use in a method of cancer treatment (e.g., acute myeloid leukemia (AML) with or without internal tandem duplication (ITD) mutations in the FLT3 gene). The ADCs of the present invention are unique therapeutic agents capable of significant tumor reduction and/or even durable complete tumor remission in AML xenograft models. Furthermore, the ADCs of the present invention when used in combination with a TKI (e.g., Midostaurin) are capable of a strong synergy with one another, which may used for treatment of FLT3-ITD+ aggressive AML.

### Definitions

### Antibodies

An “antibody” when used herein is a protein comprising one or more polypeptides (comprising one or more binding domains, preferably antigen binding domains) substantially or partially encoded by immunoglobulin genes or fragments of immunoglobulin genes. The term “immunoglobulin” (lg) is used interchangeably with “antibody” herein. The recognized immunoglobulin genes include the kappa, lambda, alpha, gamma, delta, epsilon and mu constant region genes, as well as myriad immunoglobulin variable region genes. In particular, an “antibody” when used herein, is typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2, with IgG being preferred in the context of the present invention. An antibody of the present invention is also envisaged which has an IgE constant domain or portion thereof that is bound by the Fc epsilon receptor I. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each light chain includes an N-terminal variable (V) domain (VL) and a constant (C) domain (CL). Each heavy chain includes an N-terminal V domain (VH), three or four C domains (CHs), and a hinge region. The constant domains are not involved directly in binding an antibody to an antigen, but can exhibit various effector functions, such as participation of the antibody dependent cellular cytotoxicity (ADCC). If an antibody should exert ADCC, it is preferably of the IgG1 subtype, while the IgG4 subtype would not have the capability to exert ADCC.

The term “antibody” also includes, but is not limited to, but encompasses monoclonal, monospecific, poly- or multi-specific antibodies such as bispecific antibodies, humanized, camelized, human, single-chain, chimeric, synthetic, recombinant, hybrid, mutated, grafted, and in vitro generated antibodies, with chimeric or humanized antibodies being preferred. The term “humanized antibody” is commonly defined for an antibody in which the specificity encoding CDRs of HC and LC have been transferred to an appropriate human variable frameworks (“CDR grafting”). The term “antibody” also includes scFvs, single chain antibodies, diabodies or tetrabodies, domain antibodies (dAbs) and nanobodies. In terms of the present invention, the term “antibody” shall also comprise bi-, tri- or multimeric or bi-, tri- or multifunctional antibodies having several antigen binding sites.

Furthermore, the term “antibody” as employed in the invention also relates to derivatives of the antibodies (including fragments) described herein. A “derivative” of an antibody comprises an amino acid sequence which has been altered by the introduction of amino acid residue substitutions, deletions or additions. Additionally, a derivative encompasses antibodies which have been modified by a covalent attachment of a molecule of any type to the antibody or protein. Examples of such molecules include sugars, PEG, hydroxyl-, ethoxy-, carboxy- or amine-groups but are not limited to these. In effect the covalent modifications of the antibodies lead to the glycosylation, pegylation, acetylation, phosphorylation, amidation, without being limited to these.

The antibody of the present invention is preferably an “isolated” antibody. “Isolated” when used to describe antibodies disclosed herein, means an antibody that has been identified, separated and/or recovered from a component of its production environment. Preferably, the isolated antibody is free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody will be prepared by at least one purification step.

Antibodies described herein can be used for diagnostic purposes, including sample testing and in vivo imaging, and for this purpose the antibody (or binding fragment thereof) can be conjugated to an appropriate detectable agent, to form an immunoconjugate. For diagnostic purposes, appropriate agents are detectable labels that include radioisotopes, for whole body imaging, and radioisotopes, enzymes, fluorescent labels and other suitable antibody tags for sample testing. The detectable labels can be any of the various types used currently in the field of in vitro diagnostics, including particulate labels including metal sols such as colloidal gold, isotopes, chromophores including fluorescent markers, biotin, luminescent markers, phosphorescent markers and the like, as well as enzyme labels that convert a given substrate to a detectable marker, and polynucleotide tags that are revealed following amplification such as by polymerase chain reaction. A biotinylated antibody would then be detectable by avidin or streptavidin binding. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro {1,2-dioxetane-3,2′-(5′-chloro)tricyclo{3.3.1.1 3,7}decan}-4-yl) phenyl phosphate (CSPD), as well as CDP and CDP-Star® or other luminescent substrates well-known to those in the art, for example the chelates of suitable lanthanides such as Terbium (III) and Europium (III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

Antibody “effector functions” refer to those biological activities attributable to the Fc region (a native sequence Fc region or amino acid sequence variant Fc region) of an antibody, and vary with the antibody isotype. Examples of antibody effector functions include: CIq binding and complement dependent cytotoxicity (CDC); Fc receptor binding; antibody-dependent cell-mediated cytotoxicity (ADCC); phagocytosis; down regulation of cell surface receptors (e.g., B cell receptors); and B cell activation. In order to exert effector functions an antibody, so to say, recruits effector cells.

As used herein the term “antigen binding portion” refers to a fragment of immunoglobulin (or intact antibody), and encompasses any polypeptide comprising an antigen-binding fragment or an antigen-binding domain. Preferably, the fragment such as Fab, F(ab′), F(ab′)2, Fv, scFv, Fd, disulfide-linked Fvs (sdFv), and other antibody fragments that retain antigen-binding function as described herein. Typically, such fragments would comprise an antigen-binding domain and have the same properties as the antibodies described herein. Accordingly, said fragment is preferably also capable of binding to an extracellular domain of the FLT3.

As used herein, the term “specifically binds” refers to antibodies or fragments or derivatives thereof that specifically bind to FLT3 protein and do not specifically bind to another protein. The antibodies or fragments or derivatives thereof according to the invention bind to a FLT3 protein through the variable domain of the antibody.

The pairing of a VH and VL together forms a single antigen-binding site. The CH domain most proximal to VH is designated as CH1. Each L chain is linked to an H chain by one covalent disulfide bond, while the two H chains are linked to each other by one or more disulfide bonds depending on the H chain isotype. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1 or H-CDR1 (or CRD-H1), H2 or H-CDR2 (or CDR-H2) and H3 or H-CDR3 (or CDR-H3), while CDR constituents on the light chain are referred to as L1 or L-CDR1 (or CRD-L1), L2 or L-CDR2 (or CDR-L2), and L3 or L-CDR3 (or CDR-L3).

The term “variable” refers to the portions of the immunoglobulin domains that exhibit variability in their sequence and that are involved in determining the specificity and binding affinity of a particular antibody (i.e., the “variable domain(s)”). Variability is not evenly distributed throughout the variable domains of antibodies; it is concentrated in sub-domains of each of the heavy and light chain variable regions. These sub-domains are called “complementarity determining regions” (CDRs).

The terms “CDR”, and its plural “CDRs”, refer to a complementarity determining region (CDR) of which three make up the binding character of a light chain variable region (L1-CDRL1, L2-CDR and L3-CDR) and three make up the binding character of a heavy chain variable region (H1-CDR, H2-CDR and H3-CDR). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. However, the numbering in accordance with the so-called Kabat system is preferred.

Preferred variable regions of an antibody of the present invention are shown in SEQ ID NOs: 5-18. Furhter preferred variable regions of an antibody of the present invention are shown in SEQ ID NOs: 21-62.

The more conserved (i.e., non-hypervariable) portions of the variable domains are called the “framework” regions (FRM). The variable domains of naturally occurring heavy and light chains each comprise four FRM regions, largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al., loc. cit.). The constant domains are not directly involved in antigen binding, but exhibit various effector functions, such as, for example, antibody-dependent, cell-mediated cytotoxicity and complement activation.

The term “binding domain” characterizes in connection with the present invention a domain of a polypeptide which specifically binds/interacts with a given target epitope. An “epitope” is antigenic and thus the term epitope is sometimes also referred to herein as “antigenic structure” or “antigenic determinant”. Thus, the binding domain is an “antigen-interaction-site”. The term “antigen-interaction-site” defines, in accordance with the present invention, a motif of a polypeptide, which is able to specifically interact with a specific antigen or a specific group of antigens, e.g. the identical antigen in different species. Said binding/interaction is also understood to define a “specific recognition”.

The term “epitope” also refers to a site on an antigen (in the context of the present invention, the antigen is FLT3 protein) to which the antibody molecule binds. Preferably, an epitope is a site on a molecule (in the context of the present invention, the antigen is a FLT3 protein) against which a antibody or antigen binding portion thereof, preferably an antibody will be produced and/or to which an antibody will bind. For example, an epitope can be recognized by a antibody or antigen binding portion thereof. A “linear epitope” is an epitope where an amino acid primary sequence comprises the epitope recognized. A linear epitope typically includes at least 3, and more usually, at least 5, for example, about 8 to about 10 amino acids in a unique sequence. Preferred epitopes of the present invention are SEQ ID NOs: 2-4.

The term “cross reactivity” may refer to the ability of an antibody to react with similar antigenic sites on different proteins.

The term “specifically” in this context may mean that the antibody or antigen binding portion thereof binds to target FLT3, but does not binds to another protein. The term “another protein” includes any protein including proteins closely related to or being homologous to FLT3 protein against which the antibody or antigen binding portion thereof is directed to. However, the term “another protein” does not include that the antibody or antigen binding portion thereof cross-reacts with FLT3 protein from another species different from that against which the antibody or antigen binding portion thereof was generated.

Thus, cross-species specific antibody or antigen binding portion thereof directed against FLT3 protein are preferably contemplated by the present invention.

The term “K_{D}” may refer to the equilibrium dissociation constant, a ratio of k_{off}/kₒₙ, between the antibody and its antigen or between the variable regions of one heavy and one light chain of an antibody or fragment or derivative thereof and their antigen (e.g., FLT3, and is measured in vitro. K_{D} and affinity are inversely related.

As used herein, the term “affinity” may refer to the binding strength between the variable regions of one heavy and one light chain of an antibody or fragment or derivative thereof and their antigen (e.g., FLT3, e.g., and is measured in vitro. Affinity determines the strength of the interaction between an epitope and an antibody's antigen binding site. Affinity can be calculated using the following formula:

$$KA = [ AB - AG ] / [ AB ] * [ AG ] = {k}_{on} / {k}_{off}$$

wherein:
KA=affinity constant
[AB]=molar concentration of unoccupied binding sites on the antibody
[AG]=molar concentration of unoccupied binding sites on the antigen
[AB-AG]=molar concentration of the antibody-antigen complex

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is effected e.g. by post-translational modifications like glycosylation, acetylation, phosphorylation and the like. Such modifications are well known in the art.

The term “immune cells” refers to cells which are capable of producing antibodies. The immune cells of particular interest herein are lymphoid cells derived, e.g. from spleen, peripheral blood lymphoctes (PBLs), lymph node, inguinal node, Peyers patch, tonsil, bone marrow, cord blood, pleural effusions and tumor-infiltrating lymphocytes (TIL).

A type of antibody variant encompassed by the present invention is an amino acid substitution variant. These variants have at least one, two, three, four, five, six, seven, eight, nine or ten amino acid residues in the antibody molecule replaced by a different residue. The sites of greatest interest for substitutional mutagenesis include the CDRs of the heavy and/or light chain, in particular the hypervariable regions, but FR alterations in the heavy and/or light chain are also contemplated.

For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

Generally, if amino acids are substituted in one or more or all of the CDRs of the heavy and/or light chain, it is preferred that the then-obtained “substituted” sequence is at least 60% (e.g., at least 65%, at least 70%, at least 75%, at least 80%, at least 85%, at least 90%, at least 95%, at least 96%, at least 97%, at least 98%, at least 99% or 100%), more preferably 65%, even more preferably 70%, particularly preferable 75%, more particularly preferable 80% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the antibody may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions listed in Table I, herein) is envisaged as long as the antibody retains its capability to specifically bind to FLT3 protein and/or its CDRs have an identity to the then substituted sequence (at least 60% ((e.g., at least 65%, at least 70%, at least 75%, at least 80%, at least 85%, at least 90%, at least 95%, at least 96%, at least 97%, at least 98%, at least 99% or 100%), more preferably 65%, even more preferably 70%, particularly preferable 75%, more particularly preferable 80% identical to the “original” CDR sequence).

Conservative substitutions are shown in Table I under the heading of “preferred substitutions”. If such substitutions result in a change in biological activity, then more substantial changes, denominated “exemplary substitutions” in Table I, or as further described below in reference to amino acid classes, may be introduced and the products screened for a desired characteristic.

TABLE I
Amino Acid Substitutions

| Original | Exemplary Substitutions | Preferred Substitutions |
|---|---|---|
| Ala (A) | val; leu; ile | Val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; ala; | ile |
| Lys (K) | arg; gin; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | Phe |
| Val (V) | ile; leu; met; phe; ala; | leu |

Variant: The term “variant” may refer to a polypeptide having specific activity as described herein comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

In describing the variants of the present invention, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of Asn (N) at position 167 with Thr (T) is designated as “N167T” or “Asn167Thr”. Multiple mutations can separated be addition (“+”) by marks or (“,”) e.g., “N167T+F168Y+S174C+F218Y;” or “N167T, F168Y, S174C, F218Y;”, representing multiple substitutions at given positions. In the Examples of the present application, multiple mutations can be separated by comma, e.g., N167T, F168Y, S174C, F218Y. Furthermore, “X” or “Xaa” as used herein may mean any amino acid (e.g., as depicted in Table 1 above). Accordingly, “X167T” as used herein may mean substitution of any amino acid in position 167 with T (Thr). In case where the original amino acid residue may be any amino acid residue, a short hand notation may also be used indicating only the position and substituted amino acid. Accordingly, “X” or “Xaa” may be omitted in designating substitutions, e.g., “167T” designation may be used meaning a substitution of any amino acid in position 167 with T (Thr). Furthermore, “X167G,A,S,C,U,I,L,V,T” as used herein may mean substitution of any amino acid in position 167 with any one of G, A, S, C, U, I, L, V or T. In case where the substituting amino acid residue may be any amino acid residue, a short hand notation may also be used indicating only the original amino acid and its position, e.g., “N167”.

### Chemical Modifications

The antibodies or antigen-binding variants or fragments thereof used in accordance with of the invention may be modified. Typical modifications conceivable in the context of the invention include, e.g., chemical modifications as described in the following.

Possible chemical modifications of the antibody or antigen-binding variants or fragments thereof include acylation or acetylation of the amino-terminal end or amidation or esterification of the carboxy-terminal end or, alternatively, on both. The modifications may also affect the amino group in the side chain of lysine or the hydroxyl group of threonine. Other suitable modifications include, e.g., extension of an amino group with polypeptide chains of varying length (e.g., XTEN technology or PASylation®), N-glycosylation, O-glycosylation, and chemical conjugation of carbohydrates, such as hydroxyethyl starch (e.g., HESylation®) or polysialic acid (e.g., PolyXen® technology). Chemical modifications such as alkylation (e. g., methylation, propylation, butylation), arylation, and etherification may be possible and are also envisaged.

### Antibody Drug Conjugate (ADC)

The term antibody drug conjugate (or ADC) as used herein may refer to any antibody according to present invention conjugated via its interchain disulfidebond forming cysteine residues to a phosphonamidate linked, cathepsin B cleavable monomethyl auristatin F (MMAF) cytotoxic drug having Formula I:

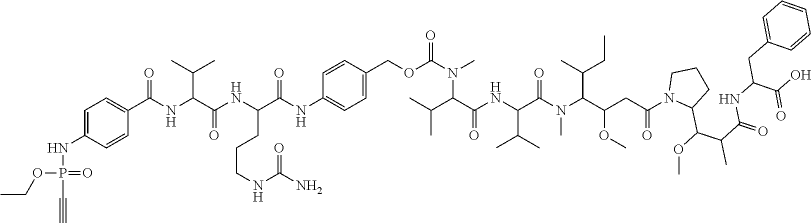 [SMILES: C#CP(=O)(Nc1ccc(C(=O)NC(C(=O)NC(CCCNC(N)=O)C(=O)Nc2ccc(COC(=O)N(C)C(C(=O)NC(C(=O)N(C)C(C(C)CC)C(CC(=O)N3CCCC3C(OC)C(C)C(=O)NC(Cc3ccccc3)C(=O)O)OC)C(C)C)C(C)C)cc2)C(C)C)cc1)OCC]

which, after the conjugation with an antibody of the present invention, may have Formula II:

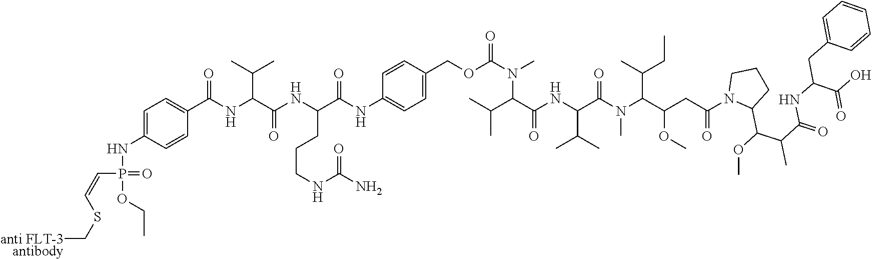

Preferably, the antibody might be conjugated to 0-8 drug molecules.

### Sequence Identity

The term “% identity” or “% sequence identity” as used herein may refer to the percentage of pair-wise identical residues-following (homologous) alignment of a sequence of a polypeptide of the invention with a sequence in question—with respect to the number of residues in the longer of these two sequences. Percent identity is determined by dividing the number of identical residues by the total number of residues and multiplying the product by 100.

The percentage of sequence homology or sequence identity can, for example, be determined herein using the BLASTP, version blastp 2.2.5 (Nov. 16, 2002; cf. Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1) including the propeptide sequences, preferably using the wild type protein scaffold as reference in a pairwise comparison. It is calculated as the percentage of numbers of “positives” (homologous amino acids) indicated as result in the BLASTP program output divided by the total number of amino acids selected by the program for the alignment.

The term “FLT3” refers to receptor-type tyrosine-protein kinase FLT3 and generally comprises all known isoforms. Preferably said FLT3 is a human FLT3 having SEQ ID NO: 1 or UniProtKB Accession Number: P36888, further preferably said extracellular domain ranges from amino acids 27 to 543 of SEQ ID NO: 1.

### Vector

The nucleic acid of the invention may also be in the form of, may be present in and/or may be part of a vector.

The term “vector” refers a nucleic acid molecule used as a vehicle to transfer (foreign) genetic material into a host cell and encompasses-without limitation-plasmids, viruses, cosmids and artificial chromosomes such as bacterial artificial chromosomes (BACs) and yeast artificial chromosomes (YACs). In general, engineered vectors comprise an origin of replication, a multicloning site and a selectable marker. The vector itself is generally a nucleotide sequence, commonly a DNA sequence that comprises an insert (transgene) and a larger sequence that serves as the “backbone” of the vector. Vectors may encompass additional elements besides the transgene insert and a backbone including gene regulation elements, genetic markers, antibiotic resistances, reporter genes, targeting sequences, or protein purification tags. Particularly envisaged within the context of the invention are expression vectors (expression constructs) for expression of the transgene in the host cell, which generally comprise—in addition to the transgene—gene regulation sequences.

An expression vector is, in general, a vector that can provide for expression of the antibodies of the present invention in vitro and/or in vivo (i.e. in a suitable host cell, host organism and/or expression system). The person skilled in the art will readily understand that choice of a particular vector include depends, e.g., on the host cell, the intended number of copies of the vector, whether transient or stable expression of the antibody of the present invention is envisaged, and so on.

“Transient expression” results from the introduction of a nucleic acid (e.g. a linear or non-linear DNA or RNA molecule) or vector that is incapable of autonomous replication into a recipient host cell. Expression of the transgene occurs through the transient expression of the introduced sequence.

However, “stable expression” of the nucleic acid sequence as described herein will often be preferred and may be accomplished by either stably integrating the nucleic acid sequence into the host cell's genome or by introducing a vector comprising the nucleic acid sequence of the invention and being capable of autonomously replicating into the host cell.

The vector provided herein is in particular envisaged to comprise a gene regulation element operably linked to the DNA sequence encoding antibody of the present invention.

The term “gene regulation element” refers to DNA sequences necessary for the expression of an operably linked coding sequence in a particular host organism. The term “gene regulation element” includes controllable transcriptional promoters, operators, enhancers, silencers, transcriptional terminators, 5′ and 3′ untranslated regions which interact with host cellular proteins to carry out transcription and translation and other elements that may control gene expression including initiation and termination codons. The precise nature of the regulatory regions needed for gene expression may vary from organism to organism. Prokaryotic gene regulation elements, for example, include a promoter, optionally an operator sequence, and a ribosome binding site (RBS), whereas gene regulation elements for eukaryotic cells comprise promoters, polyadenylation (poly-A) signals, and enhancers.

The gene regulation element is envisaged to be “operably linked” to the gene to be expressed, i.e. placed in functional relationship with the same. For instance, a promoter or enhancer is “operably linked” to a coding nucleic acid sequence if it affects the transcription of the sequence. The DNA sequences being “operably linked” may or may not be contiguous. Linking is typically accomplished by ligation at convenient restriction sites or synthetic oligonucleotide adaptors or linkers.

### Host Cell

Further provided herein is a host cell (e.g., recombinant and/or isolated host cell) comprising the vector as described herein.

A variety of host cells can be employed for expressing the nucleic acid sequence encoding antibodies as described herein. Host cells can be prepared using genetic engineering methods known in the art. The process of introducing the vector into a recipient host cell is also termed “transformation” or “transfection” hereinafter. The terms are used interchangeably herein.

Host cell transformation typically involves opening transient pores or “holes” in the cell wall and/or cell membrane to allow the uptake of material. Illustrative examples of transformation protocols involve the use of calcium phosphate, electroporation, cell squeezing, dendrimers, liposomes, cationic polymers such as DEAE-dextran or polyethylenimine, sonoporation, optical transfection, impalefection, nanoparticles (gene gun), magnetofection, particle bombardement, alkali cations (cesium, lithium), enzymatic digestion, agitation with glass beads, viral vectors, or others. The choice of method is generally dependent on the type of cell being transformed, the vector to be introduced into the cell and the conditions under which the transformation is taking place.

As used herein, the term “host cell” refers to any cell or cell culture acting as recipients for the vector or isolated nucleic acid sequence encoding the Abs as described herein. Suitable host cells include prokaryotic or eukaryotic cells, and also include but are not limited to bacteria, yeast cells, fungi cells, plant cells, and animal cells such as insect cells and mammalian cells, e.g., murine, rat, macaque or human.

E.g., the Abs can be produced in bacteria. In addition to prokaryotes, eukaryotic microbes such as filamentous fungi or yeast are suitable cloning or expression hosts for the FLT3-antibodies of the invention. Illustrative examples include Saccharomyces cerevisiae, Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of glycosylated antibody construct of the invention may also be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art.

Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR (CHO), mouse sertoli cells (TM4); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, 1413 8065); mouse mammary tumor (MMT 060562, ATCC CCL5 1); TRI cells; MRC 5 cells; FS4 cells; and human hepatoma cells (Hep G2).

### Patients

The term “patient” or “subject” as used herein refers to a human or non-human animal, generally a mammal. Particularly envisaged is a mammal, such as a rabbit, a mouse, a rat, a Guinea pig, a hamster, a dog, a cat, a pig, a cow, a goat, a sheep, a horse, a monkey, an ape or preferably a human. Thus, the methods, uses and compounds described in this document are in general applicable to both human and veterinary disease.

### Treatment

The term “treatment” in all its grammatical forms includes therapeutic or prophylactic treatment. A “therapeutic or prophylactic treatment” comprises prophylactic treatments aimed at the complete prevention of clinical and/or pathological manifestations or therapeutic treatment aimed at amelioration or remission of clinical and/or pathological manifestations of the diseases. The term “treatment” thus also includes the amelioration or prevention of cancer.

In the context with the present invention the term “therapeutic effect” in general refers to the desirable or beneficial impact of a treatment, e.g. amelioration or remission of the disease manifestations. The term “manifestation” of a disease is used herein to describe its perceptible expression, and includes both clinical manifestations, hereinafter defined as indications of the disease that may be detected during a physical examination and/or that are perceptible by the patient (i.e., symptoms), and pathological manifestations, meaning expressions of the disease on the cellular and molecular level. The therapeutic effect of treatment with the FLT3-ADC of the present invention can be assessed using routine methods in the art, e.g. measuring leukemia burden by blood/bone marrow analysis (cytomorphology, flow cytometry, genetcs), clinical chemistry or radiologic procedures (e.g. CT) Additionally or alternatively it is also possible to evaluate the general appearance of the respective patient (e.g., fitness, well-being) which will also aid the skilled practitioner to evaluate whether a therapeutic effect has been elicited. The skilled person is aware of numerous other ways which are suitable to observe a therapeutic effect of the compounds of the present invention.

### Dose

Preferably, a therapeutically effective amount of the compound as described herein is administered. By “therapeutically effective amount” is meant an amount of the compound as described herein that elicits a therapeutic effect. The exact dose of FLT3-ADC of the present invention will depend on the purpose of the treatment (e.g. remission induction, maintenance), and will be ascertainable by one skilled in the art using known techniques. Adjustments for route of administration, age, body weight, general health, sex, diet, time of administration, drug interaction and the severity of the condition may be necessary, and will be ascertainable with routine experimentation by those skilled in the art.

### Administration

A variety of routes are applicable for administration of the compound according to the present invention, including, but not limited to, orally, topically, transdermally, subcutaneously, intravenously, intraperitoneally, intramuscularly or intraocularly, preferably subcutaneously and/or intravenously. However, any other route may readily be chosen by the person skilled in the art if desired.

### Composition

It is envisaged to administer the FLT3 antibodies and/or ADCs of the present invention in the form of a pharmaceutical composition.

The term “pharmaceutical composition” particularly refers to a composition suitable for administering to a human, i.e., a composition that is preferably sterile and/or contains components which are pharmaceutically acceptable. However, compositions suitable for administration to non-human animals are also envisaged herein. Preferably, a pharmaceutical composition comprises an FLT3-ADC of the present invention together with one or more pharmaceutical excipients. The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers or tonicity modifiers. Pharmaceutical compositions of the invention can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration.

The pharmaceutical composition of the present invention may further comprise one or more additional agents. Preferably, said agents are therapeutically effective for treatment the diseases described herein and present in the composition in a therapeutically effective amount.

In view of the above, the present invention hence also provides a pharmaceutical composition comprising one or more FLT3 antibodies and/or ADCs of the present invention. Said pharmaceutical composition is particularly intended for use in a method of therapeutic and/or prophylactic treatment of cancer.

### Kit

A kit is also provided herein. The kit may be a kit of two or more parts, and comprises the FLT3 antibodies and/or ADCs of the present invention, preferably in a therapeutically effective amount and in a pharmaceutically acceptable form. The components of the kit may be contained in a container or vials. The kit is envisaged to comprise additional agents useful in treating cancer, as described elsewhere herein. Exemplary additional agents include, without limitation, a receptor tyrosine kinase inhibitor (TKI), preferably said TKI is selected from the group consisting of: Sorafenib, Midostaurin, Lestaurtinib, Sunitinib, Quizartinib, Crenolanib, Gilteritinib, TTT-3002, Tandutinib, Cabozantinib, Sel24-B489, G-749, AMG 925, FF-10101, Dovitinib, CHIR258, CHIR 258, CHIR-258, TKI258, TKI-258, TKI 258, Dovitinib DPR, Dovitinib-DRP, Mivavotinib, TAK659, TAK 659, TAK-659, CB-659, CB659, CB 659, FF-10101, SEL24, SEL 24, SEL24-B489, SEL-24, MEN1703, MEN 1703, HM43239, HM-43239, HM 43239, Luxeptinib, CG-806, CG-026806, CG026806, CG 026806, CG′806, CG 806, CG806, SKI-G-801, SKIG801, SKI G 801, Pacritinib, Enpaxiq, Epjevy, ONX-0803, ONX0803, ONX 0803, SB1518, SB1518, SB-1518, Famitinib malate, SHR-1020, SHR1020, SHR 1020, SKLB1028, SKLB-1028, SKLB 1028, Linifanib, RG3635, ABT-869, Amuvatinib, SGI-0470, MP-470, Foretinib, XL-880, XL880, GSK1363089, GSK089, ON 150030, ON150030, ON-150030, Turalio, pexidartinib, PLX108-01, PLX 108-01, PLX-108-01, PLX3397, PLX 3397, PLX-3397, [5-(5-Chloro-1H-pyrrolo[2,3-b]pyridin-3-ylmethyl)-pyridin-2-yl]-(6trifluoromethyl-pyridin-3-ylmethyl)-amine hydrochloride salt, Tandutinib, MLN0518, MLN518, CT53518, FLX925, AMG 925, FLX 92 and/or combinations thereof. Most preferably, said TKI is selected from the group consisting of: Sorafenib, Midostaurin, Lestaurtinib, Sunitinib, Quizartinib, Crenolanib, Gilteritinib.

In the course of the present invention we provide novel FLT3 targeting ADCs for AML treatment and demonstrate its efficacy in preclinical in vitro and in vivo models. To ensure circulation stability without payload-loss from the antibody, we employed the recently established P5-technology to couple the tubulin inhibitor monomethyl auristatin F (MMAF) to a human IgG1 backbone (Kasper M A, Stengl A, Ochtrop P, et al. Ethynylphosphonamidates for the Rapid and Cysteine-Selective Generation of Efficacious Antibody—Drug Conjugates. Angew Chemie—Int Ed. 2019; 58 (34): 11631-11636. doi: 10.1002/anie.201904193). This antibody scaffold maintains the ability to interact with FcgRs, especially the high affinity variant FcgR1 (CD64), which is also expressed on AML blasts and was already evaluated for targeted therapy.

In some aspects/embodiments the present invention relates to a anti-FLT3 antibody (Receptor-type tyrosine-protein kinase FLT3), wherein said anti-FLT3 antibody is capable of the following: (a) binding to an extracellular domain of said FLT3; (b) cross-reactivity with cynomolgus monkey (e.g., Macaca fascicularis) FLT3 (e.g., having UniProtKB Accession Number: Q95M30); (c) internalizing, preferably antigen-mediated antibody internalization; (d) binding to an extracellular epitope of said FLT3, wherein said extracellular epitope comprising one or more of the following sequences: SEQ ID NO: 2 (KSSSYPM); SEQ ID NO: 3 (SQGESCK); and/or SEQ ID NO: 4 (DGYP); (e) preferably said FLT3 is a human FLT3 having SEQ ID NO: 1 or UniProtKB Accession Number: P36888, further preferably said extracellular domain ranges from amino acids 27 to 543 of SEQ ID NO: 1.

In some aspects/embodiments the present invention relates to antibodies having cross-reactivity to cyno expected, as epitope 1 and 2 are identical in human and cyno, and at least 20D9 (epitope1) and 4B12 (epitope2) have been shown to be cross-reactive.

In some aspects/embodiments the present invention further relates to an antibody having the characteristics selected from the group consisting of: an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 5 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 6; an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 7 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 8; an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 9 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 10; an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 11 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 12; an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 13 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 14; an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 15 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 16; an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 17 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%) identity to SEQ ID NO: 18.

In some aspects/embodiments the present invention further relates to an antibody having the characteristics selected from the group consisting of: an antibody (e.g., 30B12) comprising a heavy chain variable region comprising heavy chain CDR1 having the amino acid sequence as set forth in SEQ ID NO: 21 (NYHVS), heavy chain CDR2 having the amino acid sequence as set forth in SEQ ID NO: 22 (AISSGGSTYYNSPLKS), and heavy chain CDR3 having the amino acid sequence as set forth in SEQ ID NO: 23 (EDGYTFGNVMDA) and a light chain variable region comprising light chain CDR1 having the amino acid sequence as set forth in SEQ ID NO: 24 (KSSQSLLYSDGKTYLN), light chain CDR2 having the amino acid sequence as set forth in SEQ ID NO: 25 (LVSKLDS), and light chain CDR3 having the amino acid sequence as set forth in SEQ ID NO: 26 (WQGTHFPYT); an antibody (e.g., 2F12) comprising a heavy chain variable region comprising heavy chain CDR1 having the amino acid sequence as set forth in SEQ ID NO: 27 (NYWMN), heavy chain CDR2 having the amino acid sequence as set forth in SEQ ID NO: 28 (QIRLKSDNYATHYAESVKG), and heavy chain CDR3 having the amino acid sequence as set forth in SEQ ID NO: 29 (SLARSDY) and a light chain variable region comprising light chain CDR1 having the amino acid sequence as set forth in SEQ ID NO: 30 (QASQNINKYIA), light chain CDR2 having the amino acid sequence as set forth in SEQ ID NO: 31 (YTSTLES), and light chain CDR3 having the amino acid sequence as set forth in SEQ ID NO: 32 (LQYVNLPRT); an antibody (e.g., 4B12) comprising a heavy chain variable region comprising heavy chain CDR1 having the amino acid sequence as set forth in SEQ ID NO: 33 (SYNVN), heavy chain CDR2 having the amino acid sequence as set forth in SEQ ID NO: 34 (AMWRGGGTDYNPALKS), and heavy chain CDR3 having the amino acid sequence as set forth in SEQ ID NO: 35 (GFGDY) and a light chain variable region comprising light chain CDR1 having the amino acid sequence as set forth in SEQ ID NO: 36 (KSSQSLKYSDGKTYLN), light chain CDR2 having the amino acid sequence as set forth in SEQ ID NO: 37 (QVSKLDS), and light chain CDR3 having the amino acid sequence as set forth in SEQ ID NO: 38 (CQGSYSPST); an antibody (e.g., 19H5) comprising a heavy chain variable region comprising heavy chain CDR1 having the amino acid sequence as set forth in SEQ ID NO: 39 (SYNVN), heavy chain CDR2 having the amino acid sequence as set forth in SEQ ID NO: 40 (AMWRGGGTDYNPALKS), and heavy chain CDR3 having the amino acid sequence as set forth in SEQ ID NO: 41 (GFGDY) and a light chain variable region comprising light chain CDR1 having the amino acid sequence as set forth in SEQ ID NO: 42 (QASQDIGNNLI), light chain CDR2 having the amino acid sequence as set forth in SEQ ID NO: 43 (CATNLAH), and light chain CDR3 having the amino acid sequence as set forth in SEQ ID NO: 44 (LQYEHYPRT); an antibody (e.g., 20D9) comprising a heavy chain variable region comprising heavy chain CDR1 having the amino acid sequence as set forth in SEQ ID NO: 45 (NYWMT), heavy chain CDR2 having the amino acid sequence as set forth in SEQ ID NO: 46 (SITKTGGGTYYPDSVKG), and heavy chain CDR3 having the amino acid sequence as set forth in SEQ ID NO: 47 (LQQLGVMDA) and a light chain variable region comprising light chain CDR1 having the amino acid sequence as set forth in SEQ ID NO: 48 (KASQNINKELN), light chain CDR2 having the amino acid sequence as set forth in SEQ ID NO: 49 (NTNNLQT), and light chain CDR3 having the amino acid sequence as set forth in SEQ ID NO: 50 (FQHKSWPLT); an antibody (e.g., 27E7) comprising a heavy chain variable region comprising heavy chain CDR1 having the amino acid sequence as set forth in SEQ ID NO: 51 (NYWMN), heavy chain CDR2 having the amino acid sequence as set forth in SEQ ID NO: 52 (QIRLKSDNYATHYAESVKG), and heavy chain CDR3 having the amino acid sequence as set forth in SEQ ID NO: 53 (SLARSDY) and a light chain variable region comprising light chain CDR1 having the amino acid sequence as set forth in SEQ ID NO: 54 (KSSQSLLYSDGKTYLN), light chain CDR2 having the amino acid sequence as set forth in SEQ ID NO: 55 (LVSKLDS), and light chain CDR3 having the amino acid sequence as set forth in SEQ ID NO: 56 (WQGTHFPYT); an antibody (e.g., 29H1) comprising a heavy chain variable region comprising heavy chain CDR1 having the amino acid sequence as set forth in SEQ ID NO: 57 (NYWMN), heavy chain CDR2 having the amino acid sequence as set forth in SEQ ID NO: 58 (QIKLKSDNYATRYAESVKG), and heavy chain CDR3 having the amino acid sequence as set forth in SEQ ID NO: 59 (SLARSDY) and a light chain variable region comprising light chain CDR1 having the amino acid sequence as set forth in SEQ ID NO: 60 (KSSQSLLHSDGKTYLN), light chain CDR2 having the amino acid sequence as set forth in SEQ ID NO: 61 (LVSKLDS), and light chain CDR3 having the amino acid sequence as set forth in SEQ ID NO: 62 (WQGTHFPYT).

In some aspects/embodiments the present invention further relates to an antibody having one or more characteristics as shown in Table 1 below:

TABLE 1
% identity of the variable regions of the light chains using NCBI
Protein Blast

| | 2F12 | 4B12 | 20D9 | 27E7 | 29H1 | 30B12 | 19H5 |
|---|---|---|---|---|---|---|---|
| 2F12 | 100.00% | 49.11% | 60.75% | 52.68% | 50.00% | 50.00% | 63.55% |
| 4B12 | 49.11% | 100.00% | 47.32% | 82.14% | 81.25% | 81.25% | 43.75% |
| 20D9 | 60.75% | 47.32% | 100.00% | 49.11% | 48.21% | 49.11% | 58.88% |
| 27E7 | 52.68% | 82.14% | 49.11% | 100.00% | 95.54% | 97.32% | 46.43% |
| 29H1 | 50.00% | 81.25% | 48.21% | 95.54% | 100% | 95.54% | 42.86% |
| 30B12 | 50.00% | 81.25% | 49.11% | 97.32% | 95.54% | 100% | 44.64% |
| 19H5 | 63.55% | 43.75% | 58.88% | 46.43% | 42.86% | 44.64% | 100% |

In some aspects/embodiments the present invention further relates to an antibody having one or more characteristics as shown in Table 2 below:

TABLE 2
% identity of the variable regions of the heavy chains using NCBI Protein Blast.

| | 2F12 | 4B12 | 20D9 | 27E7 | 29H1 | 30B12 | 19H5 |
|---|---|---|---|---|---|---|---|
| 2F12 | 100 | 48.31% | 63.33% | 94.92% | 94.07% | 48.78% | 47.22% |
| 4B12 | 48.31% | 100.00% | 52.54% | 48.31% | 49.15% | 75.83% | 99.03% |
| 20D9 | 63.33% | 52.54% | 100 | 63.33% | 62.50% | 58.33% | 51.85% |
| 27E7 | 94.92% | 48.31% | 63.33% | 100.00% | 92.37% | 47.97% | 48.15% |
| 29H1 | 94.07% | 49.15% | 62.50% | 92.37% | 100.00% | 49.59% | 48.15% |
| 30B12 | 48.78% | 75.83% | 58.33% | 47.97% | 49.59% | 100.00% | 74.55% |
| 19H5 | 47.22% | 99.03% | 51.85% | 48.15% | 48.15% | 74.55% | 100.00% |

In some aspects/embodiments the present invention further relates to an antibody having one or more characteristics as shown in Table 3 below:

TABLE 3
Examplary characteristics of the antibodies of the present invention:

| | | Epitopes | | Produ- | | | |
|---|---|---|---|---|---|---|---|
| | | (e.g., | | ction effi- | | | |
| | | Epitopes 1- | | ciancy | | | Cross |
| | Animal | 3 as defined | Affinity | (μg | Internalization in | Internalization in | reactivity |
| Ab | for | in SEQ ID | Kd | mAb/ml | MOLM- | Ba/F3- | for |
| clone | inmunization | NOs: 2-4) | (ng/ml) | culture) | 13 | FLT3 | cyno |
| 2F12 | rat | 2 | 3981 | 68 | 12% | 26% | |
| 20D9 | rat | 1 and 3 | 21.88 | 56 | 64% | 79% | yes |
| 4B12 | rat | 2 | 31.91 | 85 | 58% | 86% | yes |
| 19H5 | rat | 2 | 133.9 | 3 | | | |
| 30B12 | mouse | 1 | 3313 | 32 | | | |
| 29H1 | mouse | 1 | 28.4 | 24 | 53% | 71% | |
| 27E7 | mouse | 1 | 11.53 | 28 | 69% | 82% | |

In some aspects/embodiments the present invention further relates to an antibody drug conjugate (ADC) comprising the anti-FLT3 antibody of the present invention conjugated via its interchain disulfidebond forming cysteine residues to a phosphonamidate linked, cathepsin B cleavable monomethyl auristatin F (MMAF) cytotoxic drug. In some aspects/embodiments said drug having Formula I:

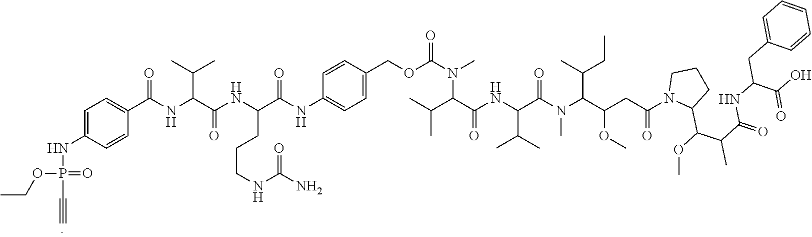 [SMILES: C#CP(=O)(Nc1ccc(C(=O)NC(C(=O)NC(CCCNC(N)=O)C(=O)Nc2ccc(COC(=O)N(C)C(C(=O)NC(C(=O)N(C)C(C(C)CC)C(CC(=O)N3CCCC3C(OC)C(C)C(=O)NC(Cc3ccccc3)C(=O)O)OC)C(C)C)C(C)C)cc2)C(C)C)cc1)OCC]

In some aspects/embodiments the antibody drug conjugate (ADC) of the present invention having Formula II:

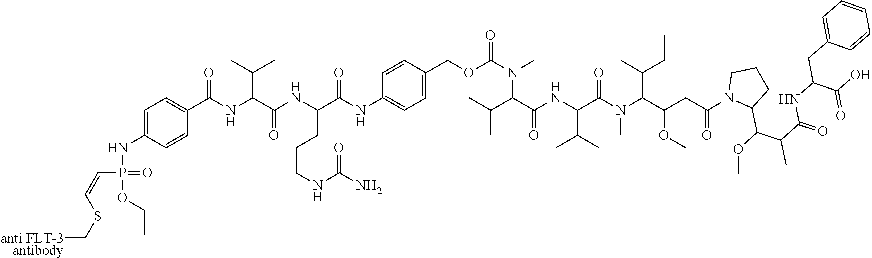

In some aspects/embodiments of the present invention, a drug to antibody ratio (DAR) of ratio is between 0 and 20, preferably between 1 and 20, further preferably between 2 and 12, most preferably between 4 and 10, further most preferably between 4 and 8.

In some aspects/embodiments of the present invention, a drug to antibody ratio (DAR) of ratio is between 1 and 20.

In some aspects/embodiments of the present invention, a drug to antibody ratio (DAR) of ratio is between 4 and 8.

In some aspects/embodiments of the present invention, a drug to antibody ratio (DAR) of ratio is between 2 and 12.

In some aspects/embodiments of the present invention, a drug to antibody ratio (DAR) of ratio is between 4 and 10.

In some aspects/embodiments of the present invention, the ADC of the present invention are capable of mediating potent cytotoxicity, e.g., to FLT3 wt and FLT3-ITD expressing Ba/F3 cell lines, AML cell lines and/or patient derived xenograft AML cells.

In some aspects/embodiments of the present invention, the ADC treatment of the present invention is capable of leading to a significant tumor reduction and/or even durable complete tumor remission, e.g., in AML xenograft models.

In some aspects/embodiments of the present invention, the ADC of the present invention demonstrate no severe hematotoxicity in in vitro colony formation assays, e.g., using concentrations which are cytotoxic in AML cell line treatment.

In some aspects/embodiments of the present invention, a combination of the ADC of the present invention with receptor tyrosine kinase inhibitor (TKI) (e.g., Midostaurin), is capable of a strong synergy in an in vitro and/or in vivo environment, which may lead to curing of FLT3-ITD+ aggressive AML cell models. This indicates that targeting of FLT3 with an advanced new generation ADC of the present invention is a promising and potent antileukemic strategy, especially when combined with FLT3-TKI in FLT3-ITD+ AML.

In some aspects/embodiments, a composition or kit of the present invention comprising one or more anti-FLT3 antibodies of the present invention and/or one or more antibody drug conjugates (ADCs) of the present invention.

In some aspects/embodiments, a composition or kit of the present invention is a pharmaceutical and/or diagnostic composition or kit, wherein said pharmaceutical or diagnostic composition further comprising a suitable receptor tyrosine kinase inhibitor (TKI), preferably said TKI is selected from the group consisting of but not exclusive: Midostaurin (PKC 412), gilteritinib, quizartinib. Any suitable TKI is encompassed by the present invention. For example, a suitable TKI can be selected from the group consisting of: Sorafenib, Midostaurin, Lestaurtinib, Sunitinib, Quizartinib, Crenolanib, Gilteritinib, TTT-3002, Tandutinib, Cabozantinib, Sel24-B489, G-749, AMG 925, FF-10101, Dovitinib, CHIR258, CHIR 258, CHIR-258, TKI258, TKI-258, TKI 258, Dovitinib DPR, Dovitinib-DRP, Mivavotinib, TAK659, TAK 659, TAK-659, CB-659, CB659, CB 659, FF-10101, SEL24, SEL 24, SEL24-B489, SEL-24, MEN1703, MEN 1703, HM43239, HM-43239, HM 43239, Luxeptinib, CG-806, CG-026806, CG026806, CG 026806, CG′806, CG 806, CG806, SKI-G-801, SKIG801, SKI G 801, Pacritinib, Enpaxiq, Epjevy, ONX-0803, ONX0803, ONX 0803, SB1518, SB1518, SB-1518, Famitinib malate, SHR-1020, SHR 1020, SHR 1020, SKLB1028, SKLB-1028, SKLB 1028, Linifanib, RG3635, ABT-869, Amuvatinib, SGI-0470, MP-470, Foretinib, XL-880, XL880, GSK1363089, GSK089, ON 150030, ON150030, ON-150030, Turalio, pexidartinib, PLX108-01, PLX 108-01, PLX-108-01, PLX3397, PLX 3397, PLX-3397, [5-(5-Chloro-1H-pyrrolo[2,3-b]pyridin-3-ylmethyl)-pyridin-2-yl]-(6 trifluoromethyl-pyridin-3-ylmethyl)-amine hydrochloride salt, Tandutinib, MLN0518, MLN518, CT53518, FLX925, AMG 925, FLX 92.

In some aspects/embodiments, the present invention relates to a method of/for treatment, amelioration, prophylaxis and/or diagnostics of cancer, preferably said cancer is acute myeloid leukemia (AML), further preferably said AML comprising internal tandem duplication (ITD) or other activating mutations in FLT3 (FLT3-ITD or other), said method comprising: administering a therapeutically or prophylactically effective amount of the antibody, antibody drug conjugate (ADC), composition and/or kit of the present invention to a subject in need thereof.

The invention is also characterized by the following items:
1. An anti-FLT3 antibody (Receptor-type tyrosine-protein kinase FLT3), preferably said FLT3 is a human FLT3 having SEQ ID NO: 1 or UniProtKB Accession Number: P36888, wherein said anti-FLT3 antibody is capable of binding to an extracellular domain of said FLT3, further preferably said extracellular domain ranges from amino acids 27 to 543 of SEQ ID NO: 1, wherein said antibody is capable of binding (e.g., specifically binding) to an extracellular epitope of said FLT3, wherein said extracellular epitope comprising one or more of the following sequences: SEQ ID NO: 2 (KSSSYPM); SEQ ID NO: 3 (SQGESCK); and/or SEQ ID NO: 4 (DGYP).
2. An anti-FLT3 antibody (Receptor-type tyrosine-protein kinase FLT3), wherein said anti-FLT3 antibody is capable of one or more of the following characteristics:
   a) binding to an extracellular domain of said FLT3;
   b) cross-reactivity with cynomolgus monkey (e.g., Macaca fascicularis) FLT3 (e.g., having UniProtKB Accession Number: Q95M30);
   c) being internalized (e.g., by target cells expressing FLT3), preferably by the means of antigen-mediated antibody internalization;
   d) binding to an extracellular epitope of said FLT3, wherein said extracellular epitope comprising one or more of the following sequences:
      i) SEQ ID NO: 2 (KSSSYPM, i.e., Epitope 1), preferably further comprising SEQ ID NO: 4 (DGYP, i.e., Epitope 3); and/or
      ii) SEQ ID NO: 3 (SQGESCK, i.e., Epitope 2), preferably further comprising SEQ ID NO: 4 (DGYP, i.e., Epitope 3);
   e) preferably said FLT3 is a human FLT3 having SEQ ID NO: 1 or UniProtKB Accession Number: P36888, further preferably said extracellular domain ranges from amino acids 27 to 543 of SEQ ID NO: 1.
3. The antibody of any one of the preceding items, wherein said antibody having (a), (b), (c) characteristics.
4. The antibody of any one of the preceding items, wherein said antibody having (a), (b), (c), (d) (i) characteristics, preferably also (e).
5. The antibody of any one of the preceding items, wherein said antibody having (a), (b), (c), (d) (ii) characteristics, preferably also (e).
6. The antibody of any one of the preceding items, wherein said antibody binding to an extracellular epitope of said FLT3, wherein said extracellular epitope comprising one or more of the following sequences: SEQ ID NO: 2 (KSSSYPM, i.e., Epitope 1) or SEQ ID NO: 3 (SQGESCK, i.e., Epitope 2).
7. The antibody of any one of the preceding items, wherein said antibody is:
   (a) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 5 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 6;
   (b) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 7 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 8;
   (c) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 9 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 10;
   (d) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 11 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 12;
   (e) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 13 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 14;
   (f) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 15 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 16; or
   (g) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 17 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 18.
   (h) wherein said antibody according to (a) is preferred.
8. The antibody of any one of the preceding items, further comprising one or more signal sequences having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NOs: 19 or 20.
9. The antibody of any one of the preceding items, wherein said antibody is a monoclonal antibody.
10. The antibody of any one of the preceding items, wherein said antibody is chimeric, humanized or human.
11. The antibody of any one of the preceding items, wherein said antibody is coupled to a labelling group.
12. The antibody of any one of the preceding items, wherein said antibody said antibody is obtainable by a hybridoma (e.g., said antibody is a recombinant antibody).
13. The antibody of any one of the preceding items, wherein said antibody having one or more of the following characteristics:
   (a) a monoclonal antibody;
   (b) a chimeric antibody and/or humanized antibody;
   (c) having K_{D} (e.g., to FLT3) in the range from about 0.1 ng/ml to about 4000 ng/ml (.e.g., from about 2 ng/ml to about 4000 ng/ml), preferably from about 10 ng/ml to about 4000 ng/ml, further preferably in the range from about 11.5 ng/ml to about 3981 ng/ml;
   (d) capable of being internalized by target cells expressing FLT3, preferably capable of being internalized by said target cells with at least 50% efficiency, further preferably capable of being internalized by said target cells with at least 80% efficiency; most preferably said internalized antibody is directed to endosomes;
   (e) a tumor-selective antibody, preferably said tumor is a liquid tumor;
   (f) a malignant-cell selective antibody.
14. The antibody according to any one of preceding items, wherein said antibody is selected from the group consisting of or corresponding to Ab clones: 20D9, 2F12, 4B12, 19H5, 30B12, 29H1 and 27E7 (e.g., as defined in the experimental section herein), preferably 20D9.
15. The antibody according to any one of preceding items, wherein said antibody having one or more of the following corresponding characteristics:

| | | Epitopes | | | | | |
|---|---|---|---|---|---|---|---|
| | | (e.g., | | | | | |
| | | Epitopes | | | | | |
| | | 1-3 as | | Production | | | |
| | | defined | | efficiancy | Internalization | Internalization | Cross |
| | Animal | in SEQ | Affinity | (μg | in | in | reactivity |
| Ab | for | ID NOs: | Kd | mAb/ml | MOLM- | Ba/F3- | for |
| clone | inmunization | 2-4) | (ng/ml) | culture) | 13 | FLT3 | cyno |
| 2F12 | rat | 2 | 3981 | 68 | 12% | 26% | |
| 20D9 | rat | 1 and 3 | 21.88 | 56 | 64% | 79% | yes |
| 4B12 | rat | 2 | 31.91 | 85 | 58% | 86% | yes |
| 19H5 | rat | 2 | 133.9 | 3 | | | |
| 30B12 | mouse | 1 | 3313 | 32 | | | |
| 29H1 | mouse | 1 | 28.4 | 24 | 53% | 71% | |
| 27E7 | mouse | 1 | 11.53 | 28 | 69% | 82% | |

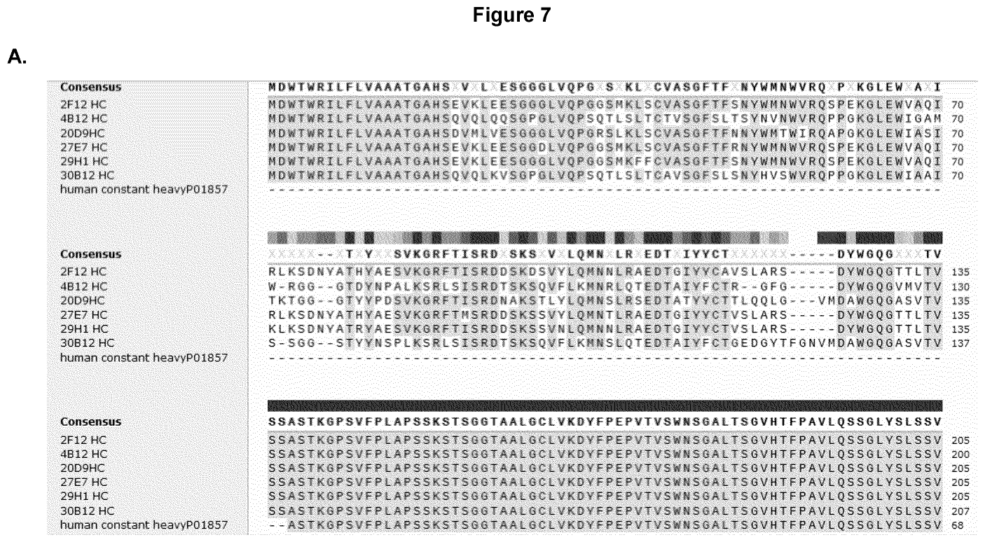

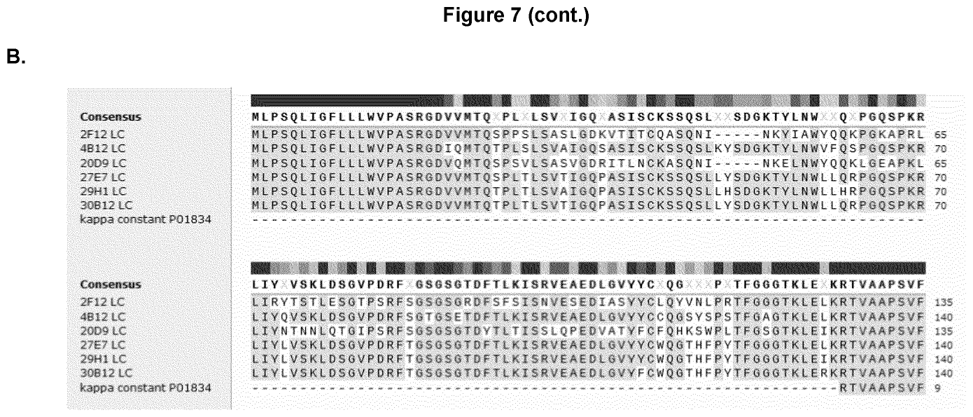

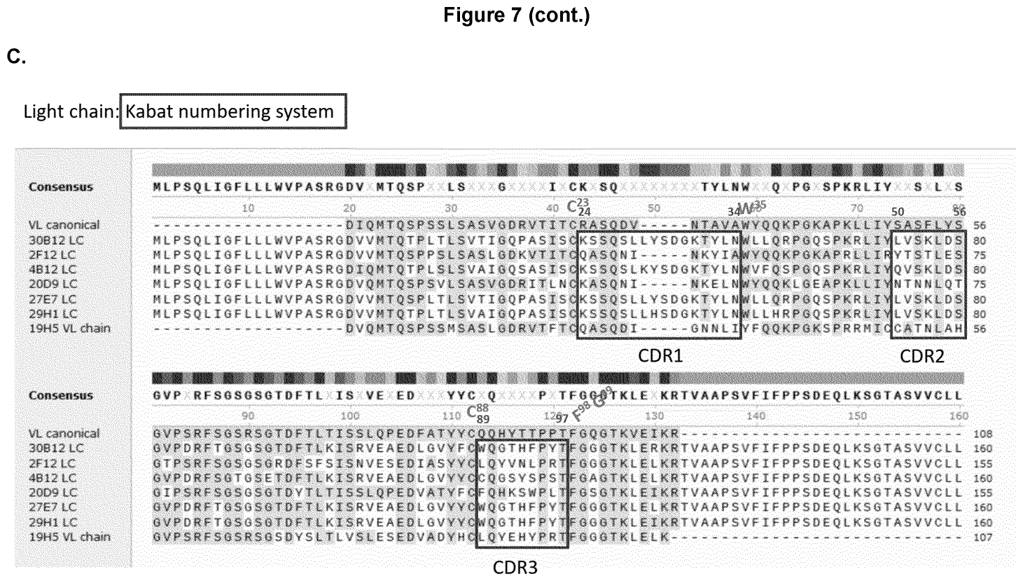

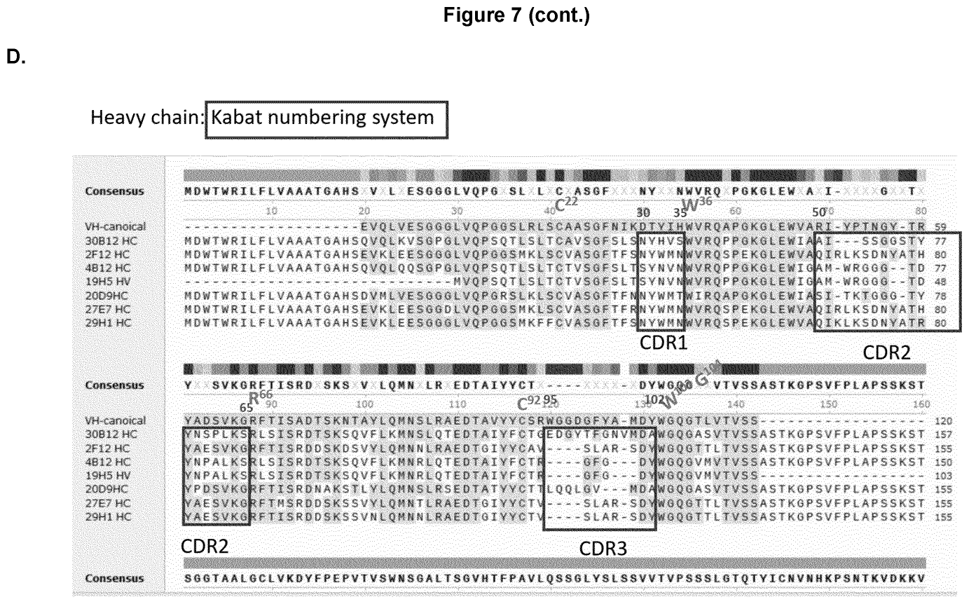

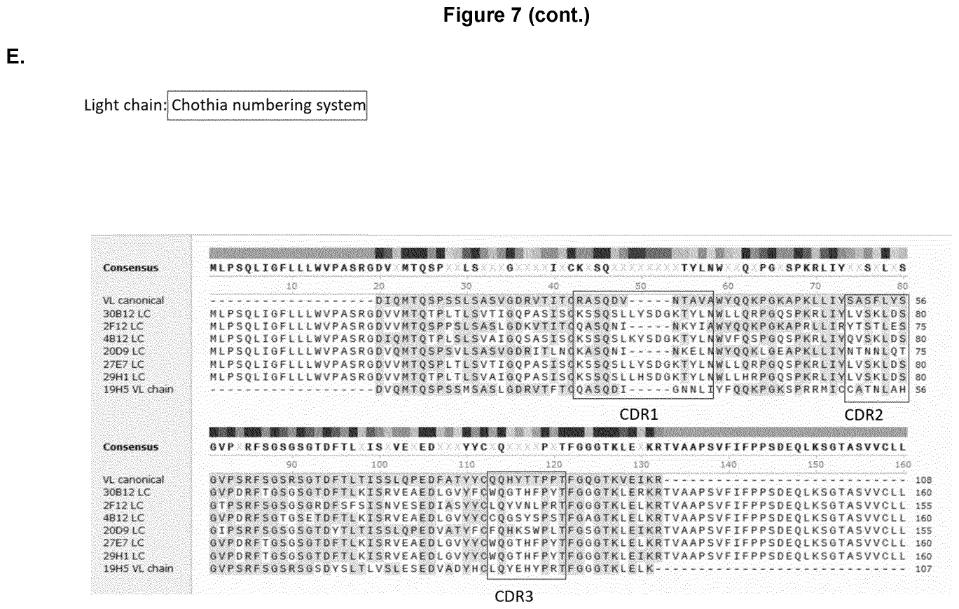

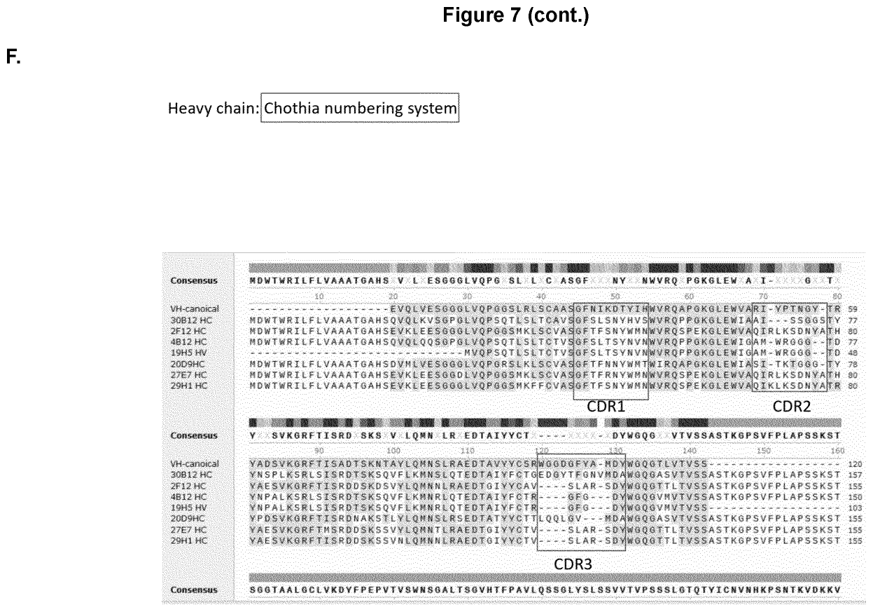

16. The antibody according to any one of preceding items, wherein said antibody comprising:
   a) one or more of the heavy chain CDRs (e.g., CDR1, CDR2 and/or CRD3) independently selected from FIG. 7 (e.g., according to Kabat numbering);
   b) one or more of the heavy chain CDRs (e.g., CDR1, CDR2 and/or CRD3) independently selected from FIG. 7 (e.g., according to Clothia numbering);
   c) one or more of the light chain CDRs (e.g., CDR1, CDR2 and/or CRD3) independently selected from FIG. 7 (e.g., according to Kabat numbering); and/or
   d) one or more of the light chain CDRs (CDR1, CDR2 and/or CRD3) independently selected from FIG. 7 (e.g., according to Clothia numbering).
17. The antibody of any one of the preceding items, wherein the antibody comprises one or more CDRs selected from the group consisting of: SEQ ID NO: 21-62.
18. The antibody of any one of the preceding items, wherein the antibody is (e.g., according to Kabat numbering):
   (a) an antibody (e.g., 30B12) comprising a heavy chain variable region comprising heavy chain CDR1 having the amino acid sequence as set forth in SEQ ID NO: 21 (NYHVS), heavy chain CDR2 having the amino acid sequence as set forth in SEQ ID NO: 22 (AISSGGSTYYNSPLKS), and heavy chain CDR3 having the amino acid sequence as set forth in SEQ ID NO: 23 (EDGYTFGNVMDA) and a light chain variable region comprising light chain CDR 1 having the amino acid sequence as set forth in SEQ ID NO: 24 (KSSQSLLYSDGKTYLN), light chain CDR2 having the amino acid sequence as set forth in SEQ ID NO: 25 (LVSKLDS), and light chain CDR3 having the amino acid sequence as set forth in SEQ ID NO: 26 (WQGTHFPYT);
   (b) an antibody (e.g., 2F12) comprising a heavy chain variable region comprising heavy chain CDR1 having the amino acid sequence as set forth in SEQ ID NO: 27 (NYWMN), heavy chain CDR2 having the amino acid sequence as set forth in SEQ ID NO: 28 (QIRLKSDNYATHYAESVKG), and heavy chain CDR3 having the amino acid sequence as set forth in SEQ ID NO: 29 (SLARSDY) and a light chain variable region comprising light chain CDR1 having the amino acid sequence as set forth in SEQ ID NO: 30 (QASQNINKYIA), light chain CDR2 having the amino acid sequence as set forth in SEQ ID NO: 31 (YTSTLES), and light chain CDR3 having the amino acid sequence as set forth in SEQ ID NO: 32 (LQYVNLPRT);
   (c) an antibody (e.g., 4B12) comprising a heavy chain variable region comprising heavy chain CDR1 having the amino acid sequence as set forth in SEQ ID NO: 33 (SYNVN), heavy chain CDR2 having the amino acid sequence as set forth in SEQ ID NO: 34 (AMWRGGGTDYNPALKS), and heavy chain CDR3 having the amino acid sequence as set forth in SEQ ID NO: 35 (GFGDY) and a light chain variable region comprising light chain CDR1 having the amino acid sequence as set forth in SEQ ID NO: 36 (KSSQSLKYSDGKTYLN), light chain CDR2 having the amino acid sequence as set forth in SEQ ID NO: 37 (QVSKLDS), and light chain CDR3 having the amino acid sequence as set forth in SEQ ID NO: 38 (CQGSYSPST);
   (d) an antibody (e.g., 19H5) comprising a heavy chain variable region comprising heavy chain CDR1 having the amino acid sequence as set forth in SEQ ID NO: 39 (SYNVN), heavy chain CDR2 having the amino acid sequence as set forth in SEQ ID NO: 40 (AMWRGGGTDYNPALKS), and heavy chain CDR3 having the amino acid sequence as set forth in SEQ ID NO: 41 (GFGDY) and a light chain variable region comprising light chain CDR1 having the amino acid sequence as set forth in SEQ ID NO: 42 (QASQDIGNNLI), light chain CDR2 having the amino acid sequence as set forth in SEQ ID NO: 43 (CATNLAH), and light chain CDR3 having the amino acid sequence as set forth in SEQ ID NO: 44 (LQYEHYPRT);
   (e) an antibody (e.g., 20D9) comprising a heavy chain variable region comprising heavy chain CDR1 having the amino acid sequence as set forth in SEQ ID NO: 45 (NYWMT), heavy chain CDR2 having the amino acid sequence as set forth in SEQ ID NO: 46 (SITKTGGGTYYPDSVKG), and heavy chain CDR3 having the amino acid sequence as set forth in SEQ ID NO: 47 (LQQLGVMDA) and a light chain variable region comprising light chain CDR1 having the amino acid sequence as set forth in SEQ ID NO: 48 (KASQNINKELN), light chain CDR2 having the amino acid sequence as set forth in SEQ ID NO: 49 (NTNNLQT), and light chain CDR3 having the amino acid sequence as set forth in SEQ ID NO: 50 (FQHKSWPLT);
   (f) an antibody (e.g., 27E7) comprising a heavy chain variable region comprising heavy chain CDR1 having the amino acid sequence as set forth in SEQ ID NO: 51 (NYWMN), heavy chain CDR2 having the amino acid sequence as set forth in SEQ ID NO: 52 (QIRLKSDNYATHYAESVKG), and heavy chain CDR3 having the amino acid sequence as set forth in SEQ ID NO: 53 (SLARSDY) and a light chain variable region comprising light chain CDR1 having the amino acid sequence as set forth in SEQ ID NO: 54 (KSSQSLLYSDGKTYLN), light chain CDR2 having the amino acid sequence as set forth in SEQ ID NO: 55 (LVSKLDS), and light chain CDR3 having the amino acid sequence as set forth in SEQ ID NO: 56 (WQGTHFPYT); or
   (g) an antibody (e.g., 29H1) comprising a heavy chain variable region comprising heavy chain CDR1 having the amino acid sequence as set forth in SEQ ID NO: 57 (NYWMN), heavy chain CDR2 having the amino acid sequence as set forth in SEQ ID NO: 58 (QIKLKSDNYATRYAESVKG), and heavy chain CDR3 having the amino acid sequence as set forth in SEQ ID NO: 59 (SLARSDY) and a light chain variable region comprising light chain CDR1 having the amino acid sequence as set forth in SEQ ID NO: 60 (KSSQSLLHSDGKTYLN), light chain CDR2 having the amino acid sequence as set forth in SEQ ID NO: 61 (LVSKLDS), and light chain CDR3 having the amino acid sequence as set forth in SEQ ID NO: 62 (WQGTHFPYT);
   (h) wherein (e) is a preferred antibody.
19. The antibody according to any one of preceding items, wherein said antibody is humanized (e.g., as in example 3 herein).
20. The antibody of any one of the preceding items, wherein said antibody is selected from the group consisting of:
   (a) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 64 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 69;
   (b) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 74 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 79;
   (c) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 84 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 89;
   (d) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 94 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 99;
   (e) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 104 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 109;
   (f) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 114 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 119;
   (g) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 124 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 129;
   (h) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 134 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 139;
   (i) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 144 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 149;
   (j) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 154 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 159;
   (k) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 164 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 169;
   (l) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 174 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 179;
   (m) an antibody comprising a heavy chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 184 and a light chain variable region having an amino acid sequence with at least 70% (e.g., at least, 80%, 85%, 86%, 87%, 88%, 89%, 90%, 91%, 92%, 93%, 94%, 95%, 96%, 97%, 98%, 99% or 100%, preferably at least 80%) identity to SEQ ID NO: 189;
   (n) preferably said antibody is according (c) or (m) (e.g., as in example 3 herein), further preferably said antibody is according (c) or (m), humanized and is capable of an increased cytotoxicity compared to parental 20D9 antibody.
21. The antibody of any one of the preceding items, wherein said antibody is selected from the group consisting of:
   (a) an antibody comprising a heavy chain variable region comprising heavy chain CDR1 having the amino acid sequence as set forth in SEQ ID NO: 65, heavy chain CDR2 having the amino acid sequence as set forth in SEQ ID NO: 66, and heavy chain CDR3 having the amino acid sequence as set forth in SEQ ID NO: 67 and a light chain variable region comprising light chain CDR1 having the amino acid sequence as set forth in SEQ ID NO: 70, light chain CDR2 having the amino acid sequence as set forth in SEQ ID NO: 71, and light chain CDR3 having the amino acid sequence as set forth in SEQ ID NO: 72;
   (b) an antibody comprising a heavy chain variable region comprising heavy chain CDR1 having the amino acid sequence as set forth in SEQ ID NO: 75, heavy chain CDR2 having the amino acid sequence as set forth in SEQ ID NO: 76, and heavy chain CDR3 having the amino acid sequence as set forth in SEQ ID NO: 77 and a light chain variable region comprising light chain CDR1 having the amino acid sequence as set forth in SEQ ID NO: 80, light chain CDR2 having the amino acid sequence as set forth in SEQ ID NO: 81, and light chain CDR3 having the amino acid sequence as set forth in SEQ ID NO: 82;
   (c) an antibody comprising a heavy chain variable region comprising heavy chain CDR1 having the amino acid sequence as set forth in SEQ ID NO: 85, heavy chain CDR2 having the amino acid sequence as set forth in SEQ ID NO: 86, and heavy chain CDR3 having the amino acid sequence as set forth in SEQ ID NO: 87 and a light chain variable region comprising light chain CDR1 having the amino acid sequence as set forth in SEQ ID NO: 90, light chain CDR2 having the amino acid sequence as set forth in SEQ ID NO: 91, and light chain CDR3 having the amino acid sequence as set forth in SEQ ID NO: 92;
   (d) an antibody comprising a heavy chain variable region comprising heavy chain CDR1 having the amino acid sequence as set forth in SEQ ID NO: 95, heavy chain CDR2 having the amino acid sequence as set forth in SEQ ID NO: 96, and heavy chain CDR3 having the amino acid sequence as set forth in SEQ ID NO: 97 and a light chain variable region comprising light chain CDR 1 having the amino acid sequence as set forth in SEQ ID NO: 100, light chain CDR2 having the amino acid sequence as set forth in SEQ ID NO: 101, and light chain CDR3 having the amino acid sequence as set forth in SEQ ID NO: 102;
   (e) an antibody comprising a heavy chain variable region comprising heavy chain CDR1 having the amino acid sequence as set forth in SEQ ID NO: 105, heavy chain CDR2 having the amino acid sequence as set forth in SEQ ID NO: 105, and heavy chain CDR3 having the amino acid sequence as set forth in SEQ ID NO: 107 and a light chain variable region comprising light chain CDR1 having the amino acid sequence as set forth in SEQ ID NO: 110, light chain CDR2 having the amino acid sequence as set forth in SEQ ID NO: 111, and light chain CDR3 having the amino acid sequence as set forth in SEQ ID NO: 112;
   (f) an antibody comprising a heavy chain variable region comprising heavy chain CDR1 having the amino acid sequence as set forth in SEQ ID NO: 115, heavy chain CDR2 having the amino acid sequence as set forth in SEQ ID NO: 116, and heavy chain CDR3 having the amino acid sequence as set forth in SEQ ID NO: 117 and a light chain variable region comprising light chain CDR1 having the amino acid sequence as set forth in SEQ ID NO: 120, light chain CDR2 having the amino acid sequence as set forth in SEQ ID NO: 121, and light chain CDR3 having the amino acid sequence as set forth in SEQ ID NO: 122;
   (g) an antibody comprising a heavy chain variable region comprising heavy chain CDR1 having the amino acid sequence as set forth in SEQ ID NO: 125, heavy chain CDR2 having the amino acid sequence as set forth in SEQ ID NO: 126, and heavy chain CDR3 having the amino acid sequence as set forth in SEQ ID NO: 127 and a light chain variable region comprising light chain CDR1 having the amino acid sequence as set forth in SEQ ID NO: 130, light chain CDR2 having the amino acid sequence as set forth in SEQ ID NO: 131, and light chain CDR3 having the amino acid sequence as set forth in SEQ ID NO: 132;
   (h) an antibody comprising a heavy chain variable region comprising heavy chain CDR1 having the amino acid sequence as set forth in SEQ ID NO: 135, heavy chain CDR2 having the amino acid sequence as set forth in SEQ ID NO: 136, and heavy chain CDR3 having the amino acid sequence as set forth in SEQ ID NO: 137 and a light chain variable region comprising light chain CDR1 having the amino acid sequence as set forth in SEQ ID NO: 140, light chain CDR2 having the amino acid sequence as set forth in SEQ ID NO: 141, and light chain CDR3 having the amino acid sequence as set forth in SEQ ID NO: 142;
   (i) an antibody comprising a heavy chain variable region comprising heavy chain CDR1 having the amino acid sequence as set forth in SEQ ID NO: 145, heavy chain CDR2 having the amino acid sequence as set forth in SEQ ID NO: 146, and heavy chain CDR3 having the amino acid sequence as set forth in SEQ ID NO: 147 and a light chain variable region comprising light chain CDR1 having the amino acid sequence as set forth in SEQ ID NO: 150, light chain CDR2 having the amino acid sequence as set forth in SEQ ID NO: 151, and light chain CDR3 having the amino acid sequence as set forth in SEQ ID NO: 152;
   (j) an antibody comprising a heavy chain variable region comprising heavy chain CDR1 having the amino acid sequence as set forth in SEQ ID NO: 155, heavy chain CDR2 having the amino acid sequence as set forth in SEQ ID NO: 156, and heavy chain CDR3 having the amino acid sequence as set forth in SEQ ID NO: 157 and a light chain variable region comprising light chain CDR1 having the amino acid sequence as set forth in SEQ ID NO: 160, light chain CDR2 having the amino acid sequence as set forth in SEQ ID NO: 161, and light chain CDR3 having the amino acid sequence as set forth in SEQ ID NO: 162;
   (k) an antibody comprising a heavy chain variable region comprising heavy chain CDR1 having the amino acid sequence as set forth in SEQ ID NO: 165, heavy chain CDR2 having the amino acid sequence as set forth in SEQ ID NO: 166, and heavy chain CDR3 having the amino acid sequence as set forth in SEQ ID NO: 167 and a light chain variable region comprising light chain CDR1 having the amino acid sequence as set forth in SEQ ID NO: 170, light chain CDR2 having the amino acid sequence as set forth in SEQ ID NO: 171, and light chain CDR3 having the amino acid sequence as set forth in SEQ ID NO: 172;
   (l) an antibody comprising a heavy chain variable region comprising heavy chain CDR1 having the amino acid sequence as set forth in SEQ ID NO: 175, heavy chain CDR2 having the amino acid sequence as set forth in SEQ ID NO: 176, and heavy chain CDR3 having the amino acid sequence as set forth in SEQ ID NO: 177 and a light chain variable region comprising light chain CDR1 having the amino acid sequence as set forth in SEQ ID NO: 180, light chain CDR2 having the amino acid sequence as set forth in SEQ ID NO: 181, and light chain CDR3 having the amino acid sequence as set forth in SEQ ID NO: 182;
   (m) an antibody comprising a heavy chain variable region comprising heavy chain CDR1 having the amino acid sequence as set forth in SEQ ID NO: 185, heavy chain CDR2 having the amino acid sequence as set forth in SEQ ID NO: 186, and heavy chain CDR3 having the amino acid sequence as set forth in SEQ ID NO: 187 and a light chain variable region comprising light chain CDR1 having the amino acid sequence as set forth in SEQ ID NO: 190, light chain CDR2 having the amino acid sequence as set forth in SEQ ID NO: 191, and light chain CDR3 having the amino acid sequence as set forth in SEQ ID NO: 192;
   (n) preferably said antibody is according (c) or (m), (e.g., as in example 3 herein), further preferably said antibody is according (c) or (m), humanized and is capable of an increased cytotoxicity compared to parental 20D9 antibody.
22. The antibody according to any one of preceding items, wherein said antibody comprises LALA mutations (e.g., Leu234Ala and Leu235Ala) in the in the Fc region (e.g., as in example 3 herein).
23. The antibody of any one of the preceding items, wherein said antibody is selected from the group consisting of:
   (a) an antibody comprising a heavy chain comprising SEQ ID NO: 63 and a light chain comprising SEQ ID NO: 68;
   (b) an antibody comprising a heavy chain comprising SEQ ID NO: 73 and a light chain comprising SEQ ID NO: 78;
   (c) an antibody comprising a heavy chain comprising SEQ ID NO: 83 and a light chain comprising SEQ ID NO: 88;
   (d) an antibody comprising a heavy chain comprising SEQ ID NO: 93 and a light chain comprising SEQ ID NO: 98;
   (e) an antibody comprising a heavy chain comprising SEQ ID NO: 103 and a light chain comprising SEQ ID NO: 108;
   (f) an antibody comprising a heavy chain comprising SEQ ID NO: 113 and a light chain comprising SEQ ID NO: 118;
   (g) an antibody comprising a heavy chain comprising SEQ ID NO: 123 and a light chain comprising SEQ ID NO: 128;
   (h) an antibody comprising a heavy chain comprising SEQ ID NO: 133 and a light chain comprising SEQ ID NO: 138;
   (i) an antibody comprising a heavy chain comprising SEQ ID NO: 143 and a light chain comprising SEQ ID NO: 148;
   (j) an antibody comprising a heavy chain comprising SEQ ID NO: 153 and a light chain comprising SEQ ID NO: 158;
   (k) an antibody comprising a heavy chain comprising SEQ ID NO: 163 and a light chain comprising SEQ ID NO: 168;
   (l) an antibody comprising a heavy chain comprising SEQ ID NO: 173 and a light chain comprising SEQ ID NO: 178;
   (m) an antibody comprising a heavy chain comprising SEQ ID NO: 183 and a light chain comprising SEQ ID NO: 188;
   (n) said antibody is according (c) or (m), (e.g., as in example 3 herein), further preferably said antibody is according (c) or (m), humanized and is capable of an increased cytotoxicity compared to parental 20D9 antibody.
24. The antibody according to any one of preceding items, wherein said antibody additionally comprises a suitable heavy chain signal sequence (e.g., SEQ ID NO: 193).
25. The antibody according to any one of preceding items, wherein said antibody additionally comprises a suitable light chain signal sequence (e.g., SEQ ID NO: 194).
26. The antibody according to any one of preceding items, wherein said antibody comprises a suitable heavy chain constant sequence and a suitable light chain constant sequence, preferably as shown in example 3 herein.
27. The antibody according to any one of preceding items, wherein said antibody comprises a suitable light chain constant sequence, preferably a human Immunoglobulin kappa constant region.
28. The antibody according to any one of preceding items, wherein said antibody comprises a suitable heavy chain constant sequence, preferably an immunoglobulin heavy constant gamma (e.g., wild type or comprising LALA mutations).
29. The antibody according to any one of preceding items, wherein said antibody comprises a sequence selected from the group consisting of: SEQ ID NOs: 63-194.
30. The antibody according to any one of preceding items, wherein said antibody is selected from the group consisting of humanized clones 1-13 of 20D9 antibody, preferably clone 3 or 13 (e.g., as in example 3 herein).
31. The antibody according to any one of preceding items, wherein said antibody is capable of binding to FLT3-ITD and/or FLT3-TKD mutated targets (e.g., mutated polypeptides), preferably said mutated FLT3 polypeptides comprising one or more of the following mutations: NPOS, D835Y, D835V, NPOS+D835Y, NPOS+N676K, NPOS+F6911 or NPOS+F691L.
32. The antibody according to any one of preceding items, wherein said FLT3-ITD and/or FLT3-TKD mutated targets (e.g., mutated polypeptides) are (e.g., compared to SEQ ID NO: 1, using the numbering of SEQ ID NO: 1):
   a) NPOS mutation comprises a 28 amino acids of SEQ ID NO: 195 (CSSDNEYFYVDFREYEYDLKWEFPRENL) inserted between L610 and E611 positions of human wild-type FLT3 polypeptide (e.g., SEQ ID NO: 1), preferably within the juxtamembrane domain of the human wild type FLT3 polypeptide;
   b) D835Y is a variant of SEQ ID NO: 1 having a substitution of D at position 835 with Y;
   c) D835V is a variant of SEQ ID NO: 1 having a substitution of D at position 835 with V;
   d) NPOS+D835Y is a variant of SEQ ID NO: 1 having the NPOS mutation/insertion of (a) and also having (b);
   e) NPOS+N676K is a variant of SEQ ID NO: 1 having the NPOS mutation/insertion of (a) also having a substitution of N at position 676 with K;
   f) NPOS+F6911 is a variant of SEQ ID NO: 1 having the NPOS mutation/insertion of (a) also having a substitution of F at position 691 with I;
   g) NPOS+F691L is the NPOS mutant of (a) also having a substitution of F at position 691 with L.
33. The antibody according to items 21 (c), 22 (c) or 23 (c) (e.g., corresponding to clone #3 as in example 3 herein), exhibiting a better IC50 in cell killing when said antibody is conjugated to MMAF compared the parental antibody 20D9 (e.g., according to item 7a (20D9), or item 18 e).
34. The antibody according to any one of preceding items, wherein said antibody is produced by immunization of an animal with FLT3 fragment of amino acids 27-541 of SEQ ID NO: 1.
35. A hybridoma, wherein said hybridoma produces the monoclonal antibody according to any one of the preceding items.
36. A nucleic acid encoding the antibody according to any one of the preceding items.
37. An expression vector comprising at least one of the nucleic acid molecules according to any one of the preceding items.
38. An isolated host cell (e.g., an isolated recombinant host cell) comprising the vector and/or nucleic acid according to any one of the preceding items.
39. An antibody drug conjugate (ADC) comprising the anti-FLT3 antibody according to any one of the preceding items conjugated via its interchain disulfidebond forming cysteine residues to a phosphonamidate linked, cathepsin B cleavable monomethyl auristatin F (MMAF) cytotoxic drug having Formula I:

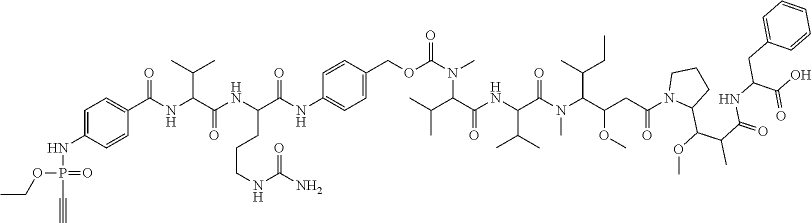 [SMILES: C#CP(=O)(Nc1ccc(C(=O)NC(C(=O)NC(CCCNC(N)=O)C(=O)Nc2ccc(COC(=O)N(C)C(C(=O)NC(C(=O)N(C)C(C(C)CC)C(CC(=O)N3CCCC3C(OC)C(C)C(=O)NC(Cc3ccccc3)C(=O)O)OC)C(C)C)C(C)C)cc2)C(C)C)cc1)OCC]

40. The antibody drug conjugate (ADC) of any one of the preceding items, wherein said ADC having Formula II:

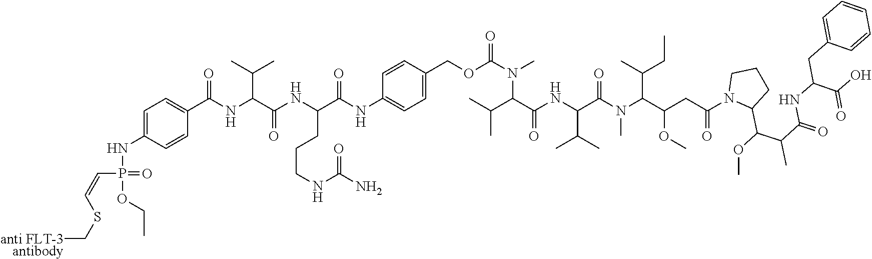

preferably to form an ADC having formula III:

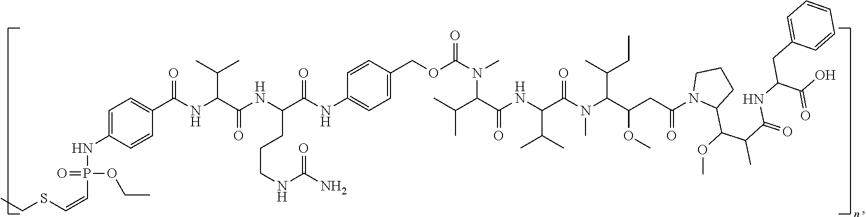

wherein n is in the range from 0 to 20 (e.g., 1, 2, 3, 4, 5, 6, 7 or 8).
41. The antibody drug conjugate (ADC) of any one of the preceding items, wherein a drug to antibody ratio (DAR) is in the range between 0 and 20, preferably is in the range between 1 and 20, further preferably is in the range between 2 and 12, most preferably is in the range between 4 and 10, further most preferably is in the range between 4 and 8.
42. The antibody drug conjugate (ADC) of any one of the preceding items, wherein the antibody is conjugated to 1 to 8 drug molecules.
43. The antibody drug conjugate (ADC) of any one of the preceding items, wherein said ADC is not glycosylated, preferably said ADC is deglycosylated.
44. A method of producing an antibody drug conjugate (ADC), said method comprising:
   (a) conjugating the antibody according to any one of the preceding items to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) cytotoxic moieties, preferably a cytotoxic drug having Formula I:

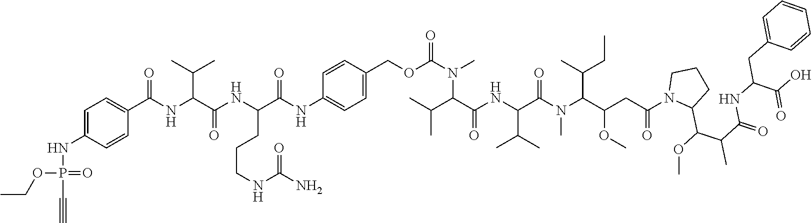 [SMILES: C#CP(=O)(Nc1ccc(C(=O)NC(C(=O)NC(CCCNC(N)=O)C(=O)Nc2ccc(COC(=O)N(C)C(C(=O)NC(C(=O)N(C)C(C(C)CC)C(CC(=O)N3CCCC3C(OC)C(C)C(=O)NC(Cc3ccccc3)C(=O)O)OC)C(C)C)C(C)C)cc2)C(C)C)cc1)OCC]

45. An antibody drug conjugate (ADC) produced by the method according to any one of the preceding items.
46. A composition or kit comprising said anti-FLT3 antibody, antibody drug conjugate (ADC), hybridoma, nucleic acid, expression vector or host cell according to any one of preceding items.
47. The composition or kit according to any one of preceding items, wherein said composition or kit is a pharmaceutical and/or diagnostic composition or kit, wherein said pharmaceutical or diagnostic composition further comprising a receptor tyrosine kinase inhibitor (TKI), preferably said TKI is selected from the group consisting of: Midostaurin (PKC 412), gilteritinib, quizartinib, Sorafenib, Midostaurin, Lestaurtinib, Sunitinib, Quizartinib, Crenolanib, Gilteritinib, TTT-3002, Tandutinib, Cabozantinib, Sel24-B489, G-749, AMG 925, FF-10101, Dovitinib, CHIR258, CHIR 258, CHIR-258, TKI258, TKI-258, TKI 258, Dovitinib DPR, Dovitinib-DRP, Mivavotinib, TAK659, TAK 659, TAK-659, CB-659, CB659, CB 659, FF-10101, SEL24, SEL 24, SEL24-B489, SEL-24, MEN1703, MEN 1703, HM43239, HM-43239, HM 43239, Luxeptinib, CG-806, CG-026806, CG026806, CG 026806, CG′806, CG 806, CG806, SKI-G-801, SKIG801, SKI G 801, Pacritinib, Enpaxiq, Epjevy, ONX-0803, ONX0803, ONX 0803, SB1518, SB1518, SB-1518, Famitinib malate, SHR-1020, SHR1020, SHR 1020, SKLB1028, SKLB-1028, SKLB 1028, Linifanib, RG3635, ABT-869, Amuvatinib, SGI-0470, MP-470, Foretinib, XL-880, XL880, GSK1363089, GSK089, ON 150030, ON150030, ON-150030, Turalio, pexidartinib, PLX108-01, PLX 108-01, PLX-108-01, PLX3397, PLX 3397, PLX-3397, [5-(5-Chloro-1H-pyrrolo[2,3-b]pyridin-3-ylmethyl)-pyridin-2-yl]-(6 trifluoromethyl-pyridin-3-ylmethyl)-amine hydrochloride salt, Tandutinib, MLN0518, MLN518, CT53518, FLX925, AMG 925, FLX 92, further preferably Sorafenib, Midostaurin (PKC 412), Lestaurtinib, Sunitinib, Quizartinib, Crenolanib, Gilteritinib, most preferably Midostaurin (PKC 412).
48. The composition or kit according to any one of preceding items, wherein a drug to antibody ratio (DAR) is in the range between 0 and 20, preferably is in the range between 1 and 20, further preferably is in the range between 2 and 12, most preferably is in the range between 4 and 10, further most preferably is in the range between 4 and 8.
49. A method for treatment, amelioration, prophylaxis or diagnostics of cancer, preferably said cancer is acute myeloid leukemia (AML), further preferably said AML comprising one or more activating mutation/s in FLT3 (e.g. FLT3-ITD, e.g., internal tandem duplication (ITD)), said method comprising: administering a therapeutically or prophylactically effective amount of the antibody, antibody drug conjugate (ADC), nucleic acid, expression vector, host cell, composition or kit according to any one of the preceding items, preferably said ADC is administered at a daily dosage of about ≤14 mg/kg, further preferably at a daily dosage of about 12 mg/kg, 10 mg/kg, 9 mg/kg, 8 mg/kg, 7 mg/kg, 6 mg/kg, 5 mg/kg, 4 mg/kg, 3 mg/kg, 2 mg/kg or 1 mg/kg, most preferably said ADC is administered at a daily dosage in the range of about 1-6 mg/kg.
50. The antibody, antibody drug conjugate (ADC), nucleic acid, expression vector, host cell, composition or kit according to any one of the preceding items, for use as a medicament and/or in therapy.
51. The antibody, antibody drug conjugate (ADC), nucleic acid, expression vector, host cell, composition or kit according to any one of the preceding items, for use in one or more of the following methods:
   (a) method for treatment, amelioration, prophylaxis or diagnostics of cancer, preferably said cancer is acute myeloid leukemia (AML), further preferably said AML comprising one or more activating mutations in FLT3 (e.g. FLT3-ITD AML);
   (b) method for monitoring development of cancer and/or for assessing the efficacy of cancer therapy;
   (c) method for screening a candidate compound for anti-cancer activity;
   (d) method for altering resistance of cancer cells to chemotherapy;
   (e) method for sensitizing cancer cells to chemotherapy;
   (f) method for inhibiting the growth of cancer cell expressing FLT3;
   (g) method for production or preparation of an antibody;
   (h) method for immunizing a non-human animal;
   (i) method for preparation of a hybridoma;
   (j) method according to any one of the preceding items;
   (k) method according to any one of (a)-(j), wherein said method is an in vivo, in vitro, or ex vivo method.
52. Use of the antibody, antibody drug conjugate (ADC), nucleic acid, expression vector, host cell, composition and/or kit according to any one of the preceding items, for one or more of the following:
   (a) for treatment, amelioration, prophylaxis and/or diagnostics of cancer, preferably said cancer is acute myeloid leukemia (AML), further preferably said AML comprising internal tandem duplication (ITD) mutations in FLT3 (FLT3-ITD AML);
   (b) for monitoring development of cancer and/or for assessing the efficacy of cancer therapy;
   (c) for screening a candidate compound for anti-cancer activity;
   (d) for altering resistance of cancer cells to chemotherapy;
   (e) for sensitizing cancer cells to chemotherapy;
   (f) for inhibiting the growth of cancer cell expressing FLT3;
   (g) for production or preparation of an antibody;
   (h) for immunizing a non-human animal;
   (i) for preparation of a hybridoma;
   (j) in a method according to any one of the preceding items; and/or
   (k) use according to any one of (a)-(j), wherein said use is an in vivo, in vitro, or ex vivo use.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., “about 20” includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be had from the following examples, offered for illustrative purposes only. The examples are not intended to limit the scope of the present invention in any way.

### EXAMPLES OF THE INVENTION

### Example 1: Targeting FLT3 by ADCs of the Present Invention in Combination with Kinase Inhibitors for Treatment of AML

### Materials and Methods:

### Cell Lines:

Cell lines were cultured according to the supplier's recommendations. For stable recombinant protein expression, Ba/F3 cells were retrovirally transduced as described before (Polzer H. et al., Exp Hematol. 2013; 41 (3): 271-280.e4.).

### Primary Samples:

Primary AML samples were obtained within trials AMLCG-99 (NCT00266136) and AMLCG-2008 (NCT01382147). Healthy bone marrow samples were obtained and isolated as described before (Wenk C, Garz A K, Grath S, et al. Blood Adv. 2018; 2 (23): 3447-3461). The study was performed in accordance with the ethical standards of the responsible committee on human experimentation (approval number LMU 068-08, LMU 222-10 and TUM 538/16) and with the Helsinki Declaration of 1975, as revised in 2013.

### Binding and Internalization of Monoclonal Antibodies

For antibody binding studies, cells were stained on ice with primary mouse, rat or human anti-FLT3 antibodies (in house) and secondary antibody Goat F(ab′)2 Anti-Human Ig-PE (2012-09), Goat Anti-Mouse IgG (H+L)-PE (1032-09) or Goat Anti-Rat IgG (H+L)-PE (3052-09) purchased from Southern Biotech. For internalization experiments, cells incubated with anti-FLT3 antibodies were washed and incubated for 30 min at 4° C. or 37° C. followed by staining with secondary antibody.

### Cytotoxicity Proliferation Assays

Suspension cells were treated with 20D9-ADC or IgG1-ADC (in-house), deglycosylated ADC generated by applying Endo S (P0741L NEB), 20D9 mab (in house), Synagis (Palivizumab 404770, AbbVie), Quizartinib (S1526, Selleck Chem) or Midostaurin (MedChemExpress). AML cells were treated once (d0) and viability was determined after 96h using Resazurin solution (50 UM final concentration, 4 h incubation) (R12204, Thermo Fisher Scientific). For Ba/F3 cell assays, cells were treated once (d0) and viable cells were counted after 72 h on Vi-Cell Cell Viability Analyzer (Beckman Coulter, Krefeld, Germany). Calculation of IC50 values were performed using GraphPad Prism version 6.07 (GraphPad Software, La Jolla, CA, USA).

### In Vivo Experiments

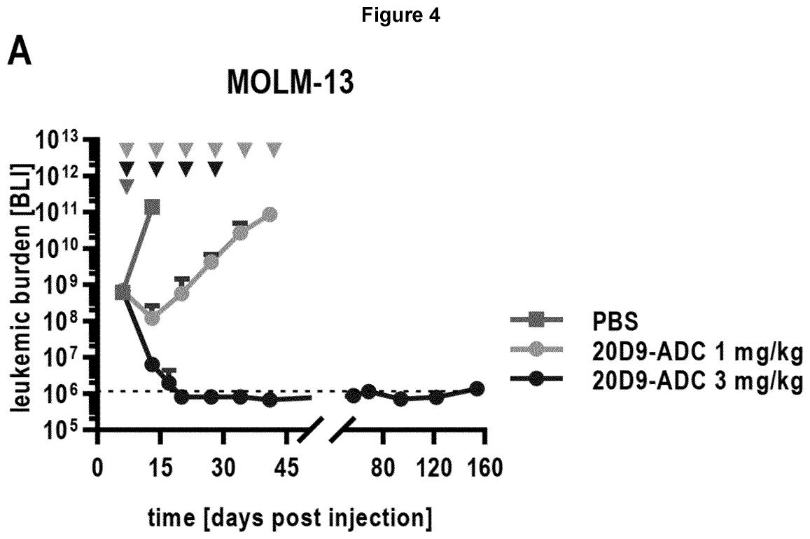

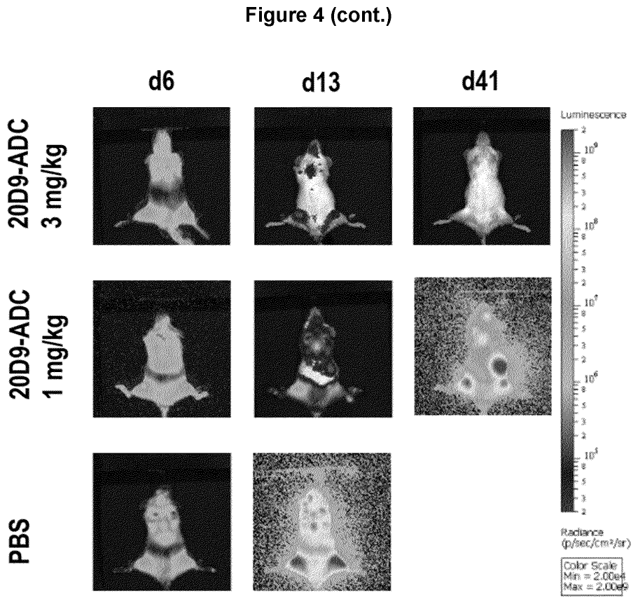

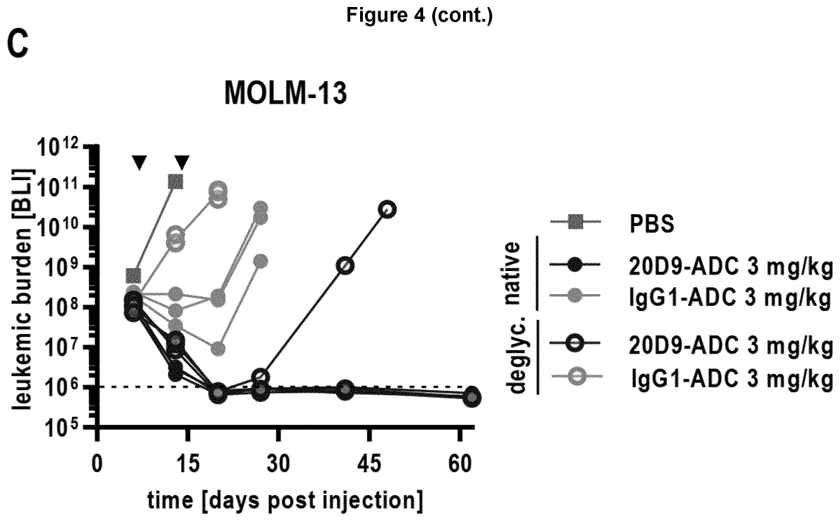

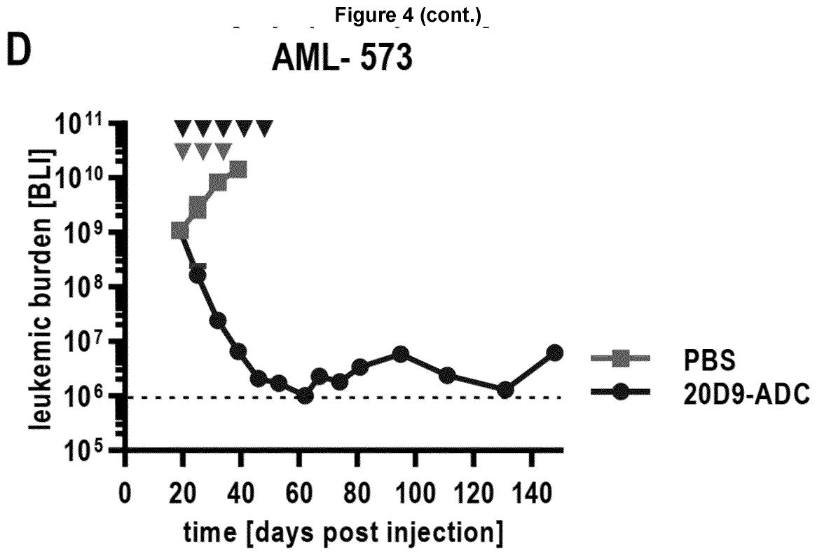

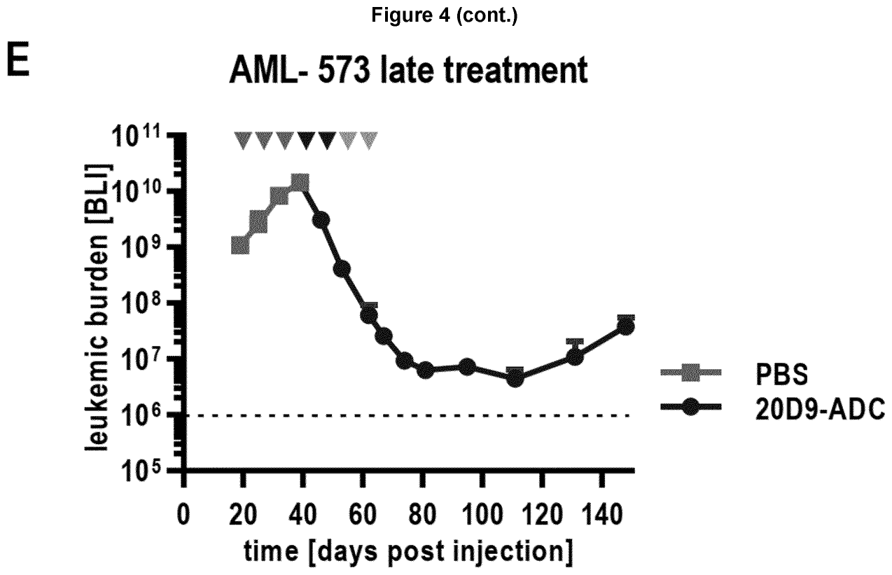

Patient-derived xenograft (PDX) cells or MOLM-13 cells expressing enhanced firefly luciferase and mCherry (Addgene, Plasmid #104833) were established as described previously (Vick B, Rothenberg M, Sandhöfer N, et al. PLOS One. 2015; 10 (3)). For in vivo therapy trials, MOLM-13 cells or PDX cells were injected intravenously (i.v.) into 8-12 week old male NSG mice (NOD scid gamma, The Jackson Laboratory, Bar Harbour, ME, USA), and tumor growth was regularly monitored by bioluminescence imaging (BLI) as described previously by Vick et al. 2015. After successful engraftment, mice were treated with deglycosylated or native 20D9-ADC (1 or 3 mg/kg, i.v., 1 dose per week), IgG1-ADC (3 mg/kg), or Midostaurin (SelleckChem, 50 mg/kg, oral gavage, 5 doses per week; in 5% DMSO+45% PEG300+50% ddH₂O). Experimental end points were BLI values above 1×10¹⁰ Photons/see or below detection limit (4×10⁶ Photons/sec) for 90-150 days post injection. Mice showing clinical signs of illness or weight loss above 15% under therapy were sacrificed (1 ADC treated mouse in FIG. 4D). Mice which died in inhalation narcosis were excluded from further analyses (1 ADC treated mouse in FIG. 4D). All animal trials were performed in accordance with the current ethical standards (Regierung von Oberbayern, number ROB-55.2Vet-2532.Vet_02-16-7).

### Results

### Generation and Characterization of Anti-FLT3 Antibodies

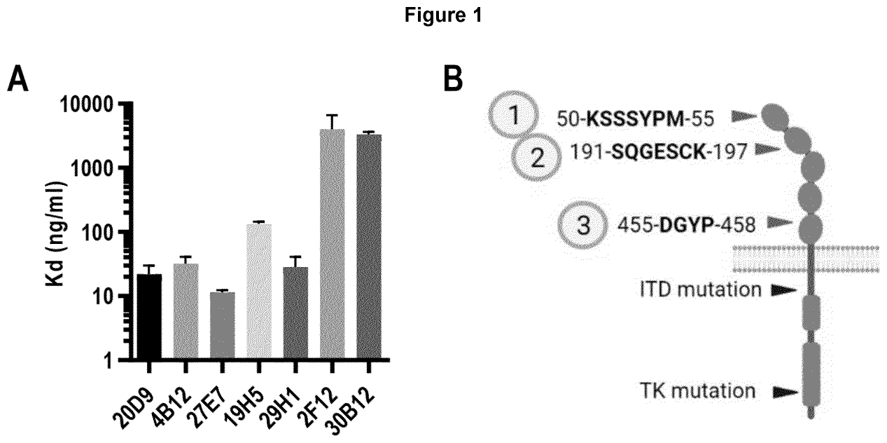

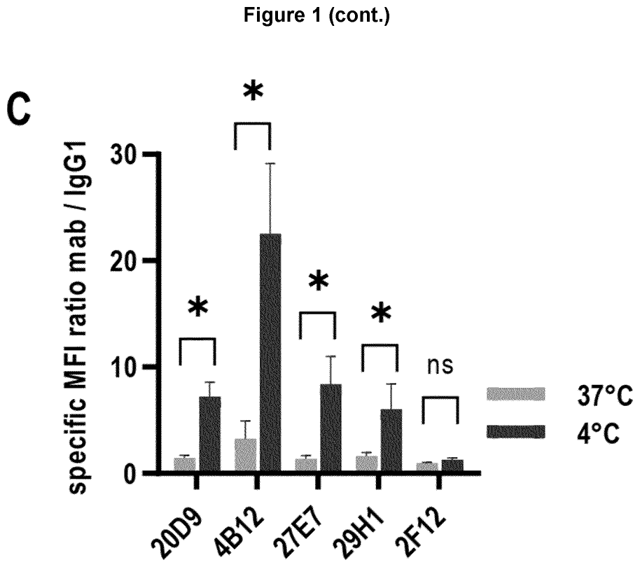

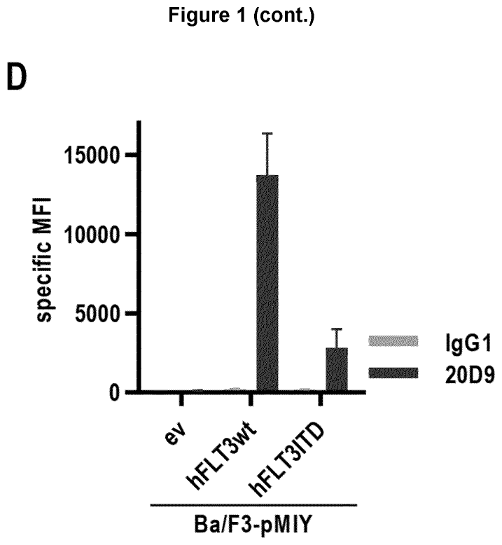

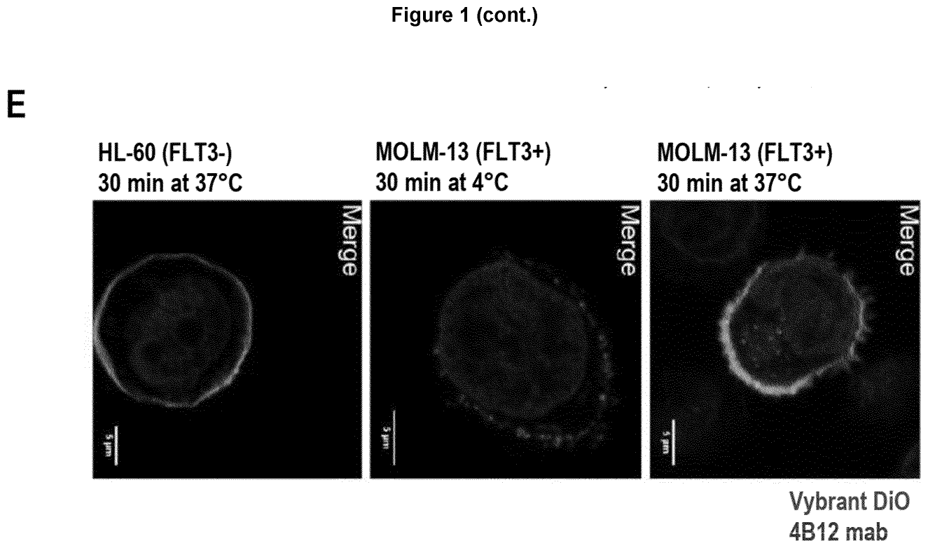

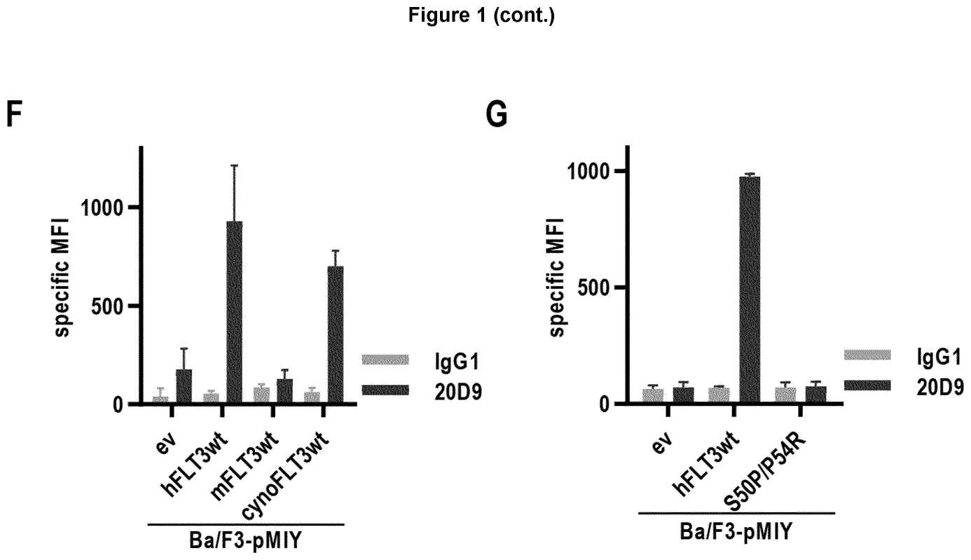

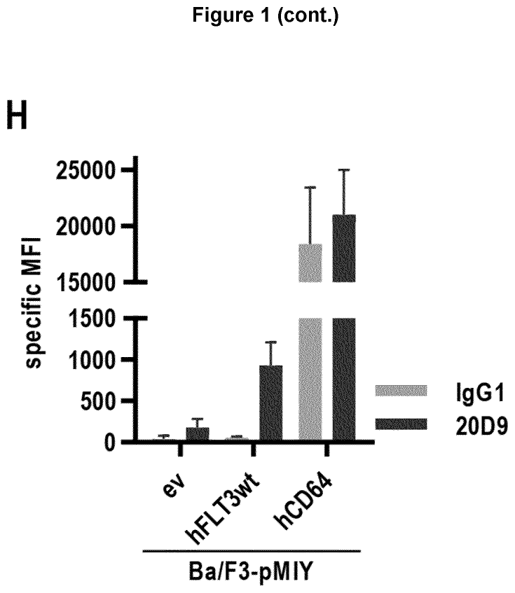

Monoclonal FLT3 specific antibodies were generated by hybridoma cells of isolated B cells from immunized rats and mice. After selection procedures, seven antibodies were chimerized using a human IgG1 sequence. Chimeric antibodies were efficiently expressed in HEK cells and possessed high protein stability. Binding affinities to recombinant FLT3 protein varied from K_{D}=11.5 ng/ml to Kd=3981 ng/ml between the different clones (FIG. 1A).

Epitope mapping of the antibodies to peptides derived from extracellular domain of FLT3 identified two main binding motifs KSSSYPM (SEQ ID NO: 2) (bound by 30B12, 29H1, 27E7, 20D9) and SQGESCK (SEQ ID NO: 3) (bound by 19H5, 4B12, 2F12) (FIG. 1B). The rat 20D9 showed additional affinity to a third minor epitope DGYP (SEQ ID NO: 4). To identify their suitability for ADC development, the binding and internalization efficiencies of the antibodies to Ba/F3 cell line stably expressing human FLT3 or empty MSCV-IRES-YFP vector (pMIY) were evaluated. The clones 20D9, 4B12, 29H1 and 27E7 specifically bound Ba/F3-FLT3 cell line by flow cytometry (FIG. 1C) and 20D9 showed specific binding to Ba/F3 cell lines expressing FLT3-ITD (FIG. 1D). Further, the antibodies showed significant internalisation of around 80% in flow cytometry-based internalisation assays in Ba/F3-FLT3 cell line (FIG. 1C). These observations could be confirmed in AML cells lines in flow cytometry and immune fluorescent staining (FIG. 1E). Further, the internalized antibodies were directed to endosomes, which was demonstrated by the co-localization of the antibody and EEA1. Based on internalization, and high expression yields we selected the 20D9 clone for further development and evaluated the binding to FLT3 ortologs from different species. The protein sequence of human FLT3 in epitope 1 is identical to the cynomolgus FLT3 and differs to the murine FLT3 receptor. In contrast to the murine FLT3, the cynomolgus monkey FLT3 expressing Ba/F3 cell lines bound the 20D9 antibody (FIG. 1F). To proof the epitope specificity, we expressed a human FLT3 receptor with the epitope region mutated to the murine variant (S50P/P54R) in Ba/F3 cells, which were not able to bind the 20D9-mab (FIG. 1G). Finally, we verified the binding of 20D9-mab to the high affinity Fc receptor CD64 via the IgG1 backbone using Ba/F3 cell lines expressing human CD64 (FIG. 1H).

### Generation and Characterization of New Generation 20D9-Antibody Drug Conjugate

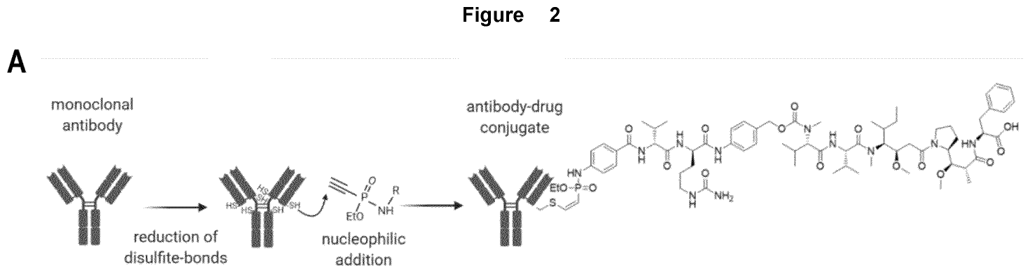

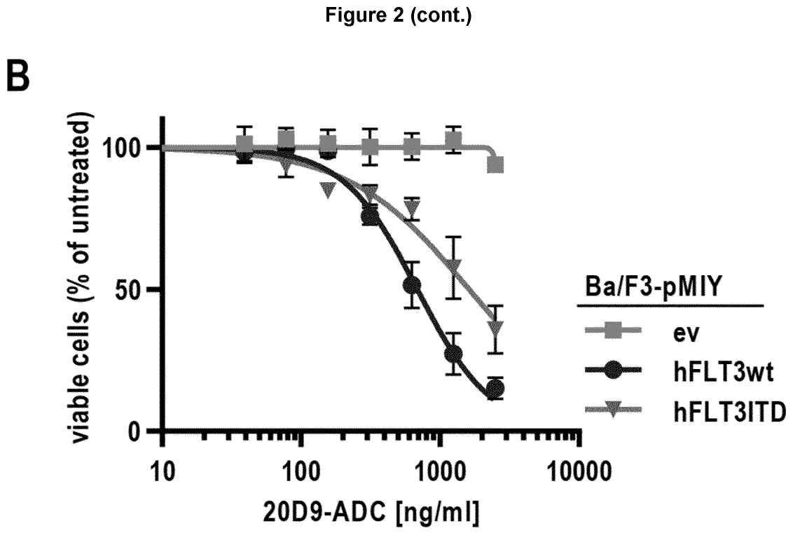

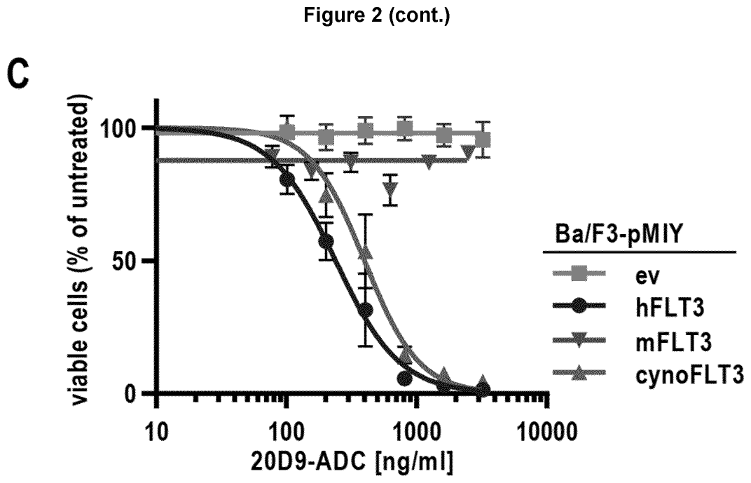

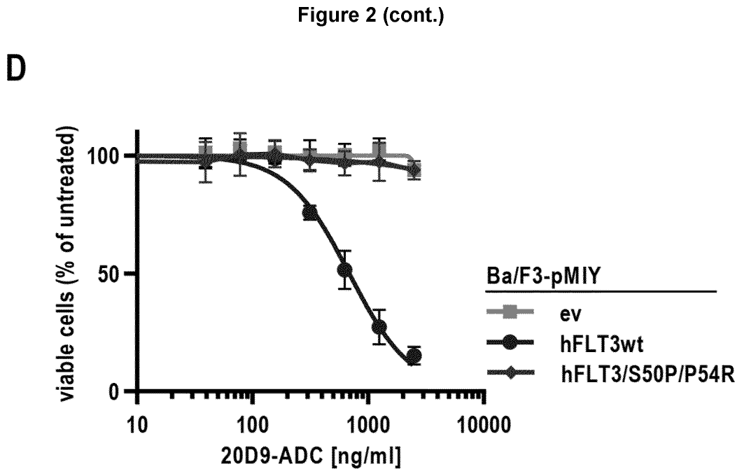

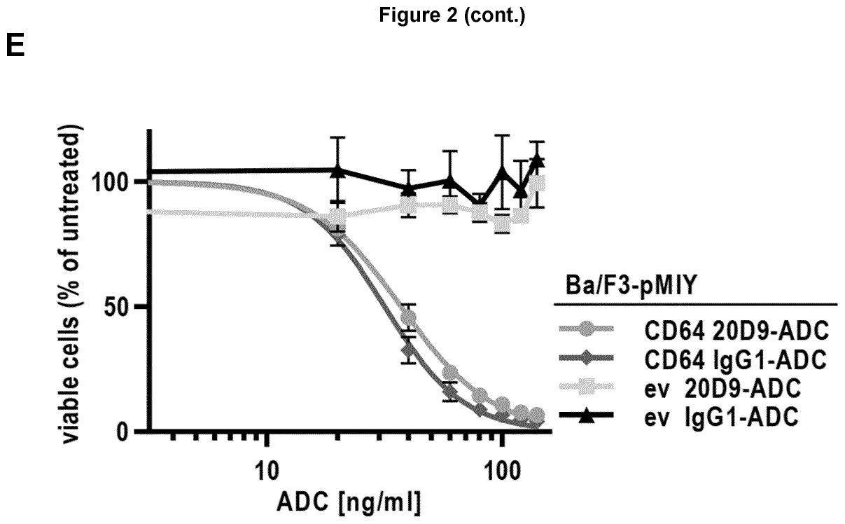

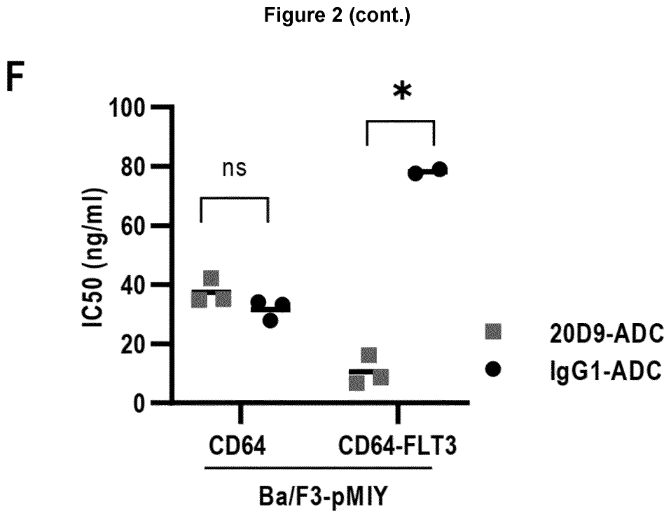

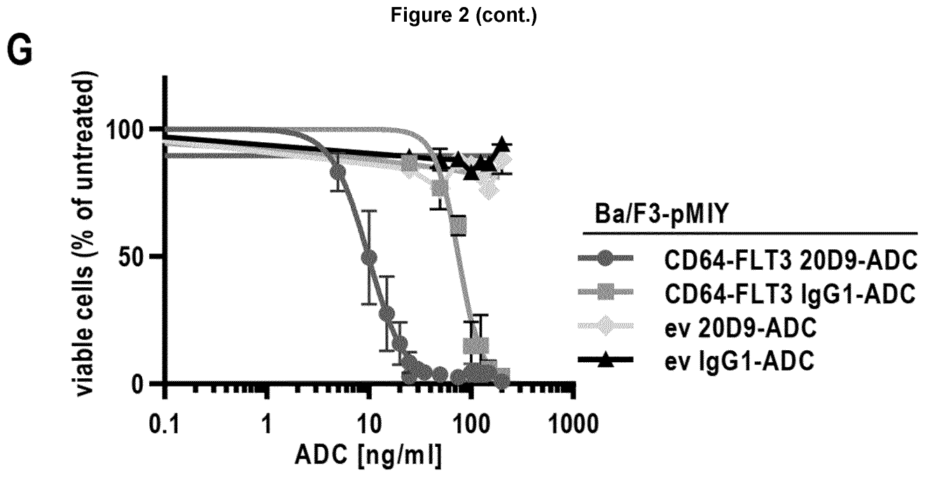

We applied the recently developed P5-technology, which uses Ethynylphosphonamidates for a stable conjugation to the antibodies' cysteine residues. We conjugated IgG1 based 20D9 with the tubulin polymerization inhibitor monomethylauristatin F (MMAF) payload with a drug to antibody ratio (DAR) of ratios between 4 and 8 via a cathepsin B cleavage side (FIG. 2A). An incubation for 2 weeks at 40° C. or 14-month storage at 4° C. did not impact the 20D9 antibody-toxin conjunction and only slightly enhanced the aggregation. To obtain an IgG1-ADC only possessing the CD64 but no FLT3 binding, we conjugated MMAF to the IgG1 based antibody palivizumab37, which is specific for the glycoprotein F of the Respiratory Syncytial Virus 38. In cytotoxicity assays, human wildtype, human ITD mutated (FIG. 2B) as well as cynomolgus FLT3 (FIG. 2C) expressing Ba/F3 cell lines were sensitive to 20D9-ADC treatment. Consistent with the binding analysis, 20D9-ADC was not cytotoxic in murine FLT3 and epitope mutant human FLT3 S50P/P54R expressing Ba/F3 cell lines (FIG. 2C,D). As controls we used MMAF and control IgG1-ADC in Ba/F3 cells expressing the empty vector or human wild type FLT3 which showed no difference in cytotoxicity and no cytotoxicity, respectively. Further, we assessed the cytotoxicity mediated by the IgG1-FcR binding of the 20D9-ADC. Ba/F3-pMIY-CD64 expressing cells were sensitive to 20D9-ADC and control IgG1-ADC with similar IC50 values of 37.3 and 31.8 ng/ml, respectively (FIG. 2E,F). Ba/F3 cell lines expressing CD16 or CD32 did not respond to 20D9-ADC. Ba/F3 cells expressing both FLT3 and CD64 were significantly more sensitive (IC₅₀=0.5 ng/ml) to 20D9-ADC compared the control IgG-ADC (IC₅₀=78.3 ng/ml), indicating the advantage of targeting both antigens in vitro (FIG. 2F,G).

### In Vitro Cytotoxic Activity of 20D9-ADC in AML Cell Lines

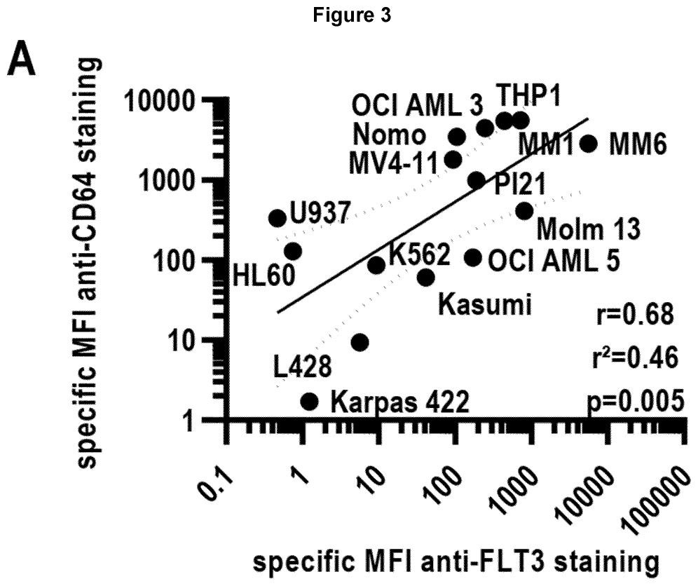

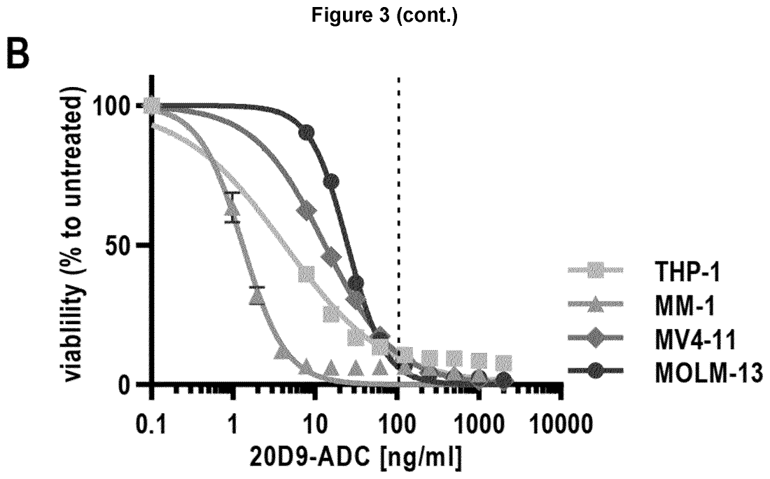

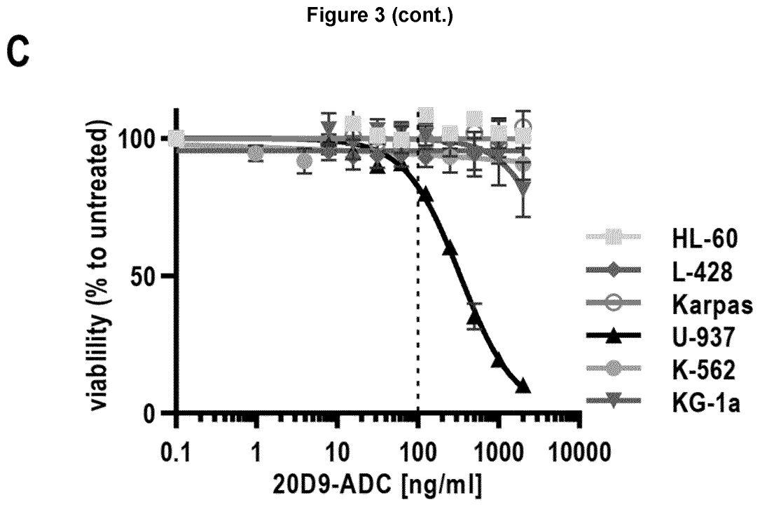

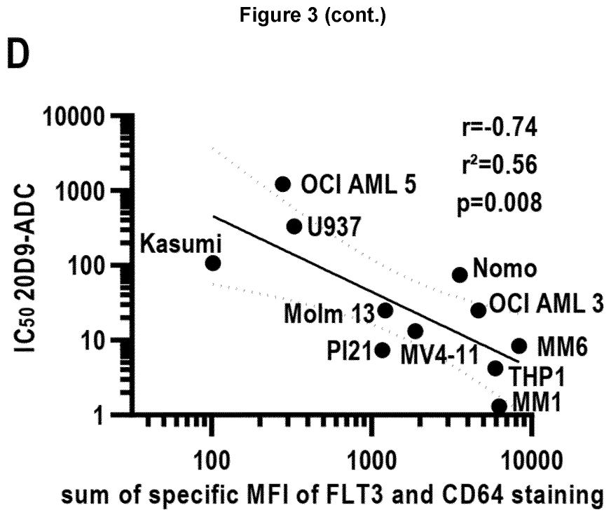

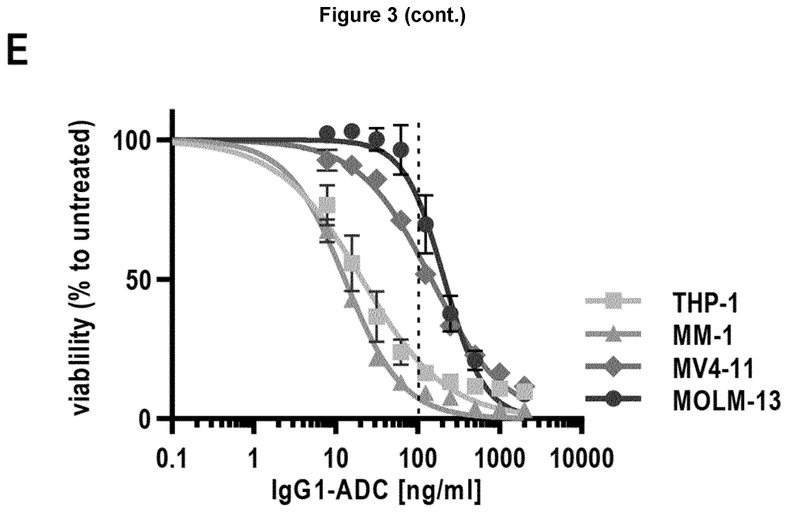

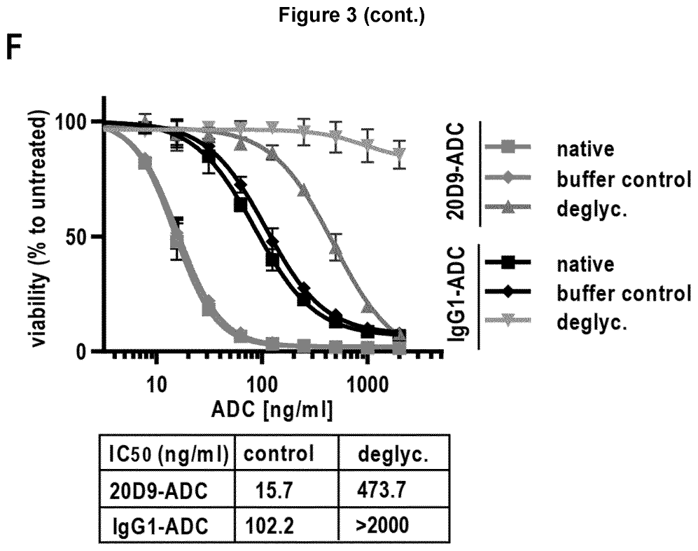

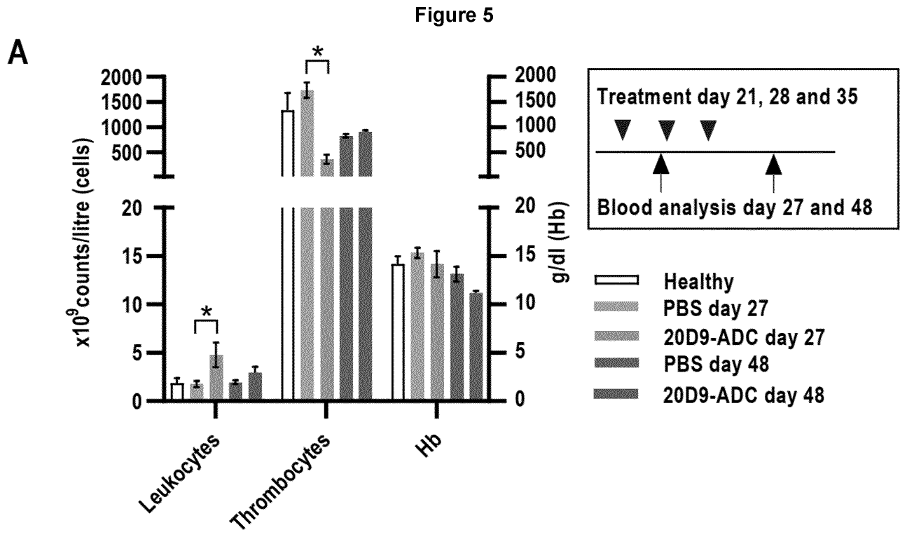

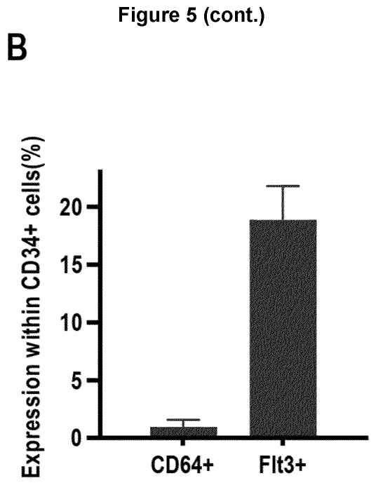

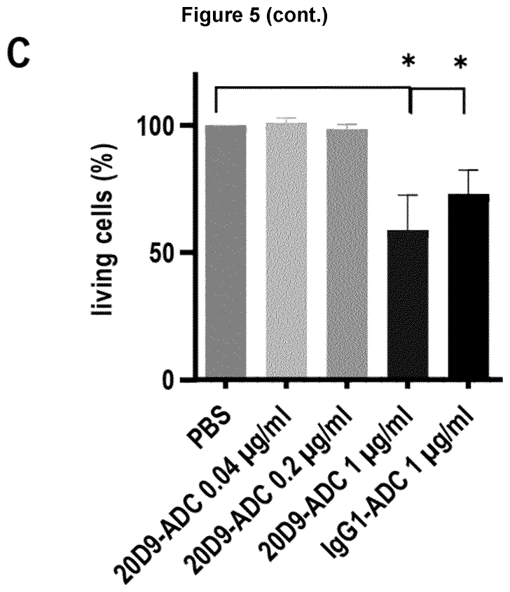

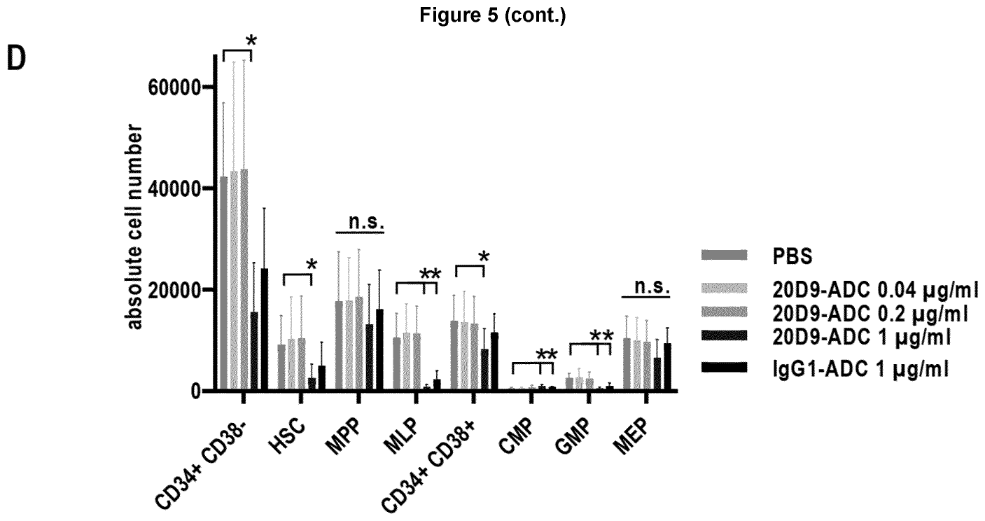

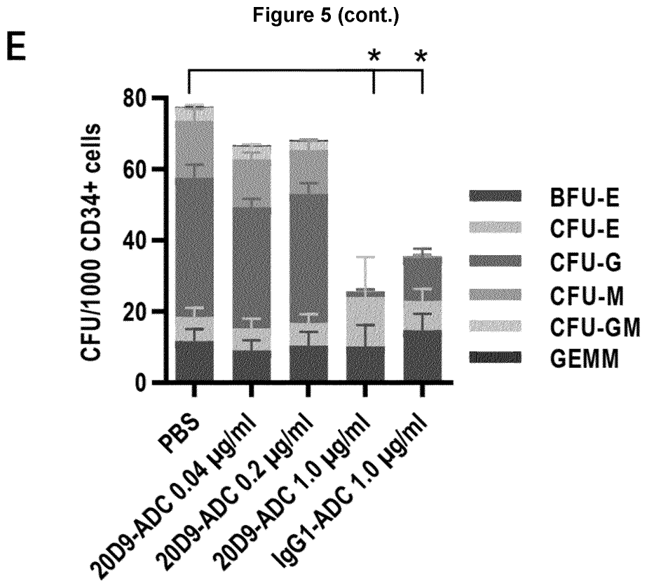

We determined the FLT3 and CD64 expression in different leukemia and lymphoma cell lines and could detect a significant correlation of both expression levels (FIG. 3A). Binding of the 20D9 antibody and cytotoxicity of the 20D9-ADC could be shown in all FLT3 positive cell lines with IC₅₀ values varying between 1.3 and 107.33 ng/ml (FIG. 5B). The 20D9-ADC acts via apoptosis induction, which was demonstrated in MOLM-13 cells compared to the FLT3 negative cell line HL-60. Further, we observed no cytotoxicity in 5 out of 6 FLT3 negative AML cell lines. The FLT3-negative, CD64 positive cell line U-937 showed an IC50 value of 334 ng/ml (FIG. 3C). Consistently, there was a correlation of summarized CD64 and FLT3 expression and the 20D9-ADC IC50 value (FIG. 3D). The IgG1-ADC showed cytotoxic activity in all CD64 positive cell lines (FIG. 3E). The IC50 values ranged from 12.82 to around 2000 ng/ml and were therefore significantly higher compared to 20D9-ADC. FLT3 positive and negative cell lines showed similar sensitivity towards the payload MMAF and the native 20D9 antibody or the control IgG1 antibody (data not shown) did not impair cell proliferation. To investigate the impact of CD64 interaction on the efficacy of IgG1 based ADCs, we disrupted the CD64-IgG1 binding by removing the N-linked glycans of 20D9-ADC and IgG1-ADC. Compared to the native 20D9-ADC, the IC50 value of deglycosylated 20D9-ADC shifted from 15.7 ng/ml to 473.7 ng/ml (FIG. 3F), reflecting the FLT3-specific targeting. As control, the deglycosylated IgG1-ADC showed no activity on MOLM-13 cells, confirming the effective abrogation of the CD64-FcR interaction (FIG. 3F).

### Antileukemic Activity of 20D9-ADC in Cell Line and Patient Derived Xenograft AML Mouse Models

To determine the in vivo antileukemic activity of 20D9-ADC, we used xenograft mouse models of AML combined with bioluminescence imaging (BLI) for monitoring of treatment effects. Transgenic cells expressing a luciferase were injected into immunodeficient NSG mice followed by weekly intravenous ADC administration after positive engraftment. First, we analysed the efficacy of ADCs in the MOLM-13 xenograft model in vivo. While repetitive administration of 1 mg/kg (Q1W×6) 20D9-ADC decelerated the increase of BLI signal compared to PBS treated control mice, 3 mg/kg (Q1W×4) led to a strong reduction of the tumor burden below BLI detection limit for at least 154 days (FIG. 4A,B). The anti-tumor effect was comparable if 3 mg/kg therapy started at intermediate or advanced, tumor burden. To define CD64-related effects of the 20D9-ADC, we applied native and deglycosylated IgG1-ADC and 20D9-ADC (FIG. 4C). Interestingly, the deglycosylated 20D9-ADC showed a strong cytotoxicity comparable to the native 20D9-ADC, indicating that FLT3 targeting is sufficient to elicit a long-lasting durable response. In contrast, the anti tumor effect of IgG1-ADC (3 mg/kg; Q1W×2) was reduced compared to native 20D9-ADC underlining that CD64 targeting is less effective in vivo. The deglycosylated IgG1-ADC had only a minimal effect compared to PBS control mice, confirming the successful deglycosylation (FIG. 4C). Next, we determined the effect of 20D9-ADC on patient derived xenograft (PDX) samples. We selected patient derived samples with FLT3-ITD mutation and moderate to high FLT3 expression compared to human primary samples. Ex vivo, the cells were sensitive towards 20D9-ADC treatment while the 20D9-mab remained ineffective. In vivo treatment of AML-573 transplanted mice with 3 mg/kg 20D9-ADC (Q1W×5) led to a strong tumor reduction followed by stable low tumor burden up to 150 days, both if treatment started at mediumor advanced (FIG. 4D,E) tumor burden. Similarly, in two additional PDX samples, AML-640 and AML-579, treatment with 3 mg/kg 20D9-ADC (Q1W×3) at intermediate or advanced tumor burden led to a strong tumor reduction followed by a tumor outgrowth after treatment stop.

### Hematotoxicity of 20D9-ADC.

To assess hematotoxicity we analysed blood counts of 20D9-ADC (3 mg/kg) and control treated mice. We found a moderate thrombocytopenia on day 27 (364 g/l and 1736 g/l respectively) and a significant increase of leukocyte count from 1.8 to 4.78 g/l already after one administration. Both levels were recovered at day 48. The haemoglobin levels did not change during treatment (FIG. 5A). Further, we investigated the effect of the 20D9-ADC on normal human hematopoiesis in vitro by analysing bone marrow cells from healthy donors enriched for CD34 expression. As expected, FLT3 expression could be detected in 18.9% (±2.9%) of CD34+cells, while CD64 was barely expressed (1% (±0.6%), FIG. 5B). Treatment for 4 days with 20D9-ADC in concentrations in the range of IC50 values in AML cells (40 and 200 ng/ml) did not show a significant toxicity in CD34+ cells compared to the untreated control. However, a high dose of 20D9-ADC or IgG1-ADC (1000 ng/ml) led to significantly decreased cell viability (FIG. 5C) which might indicate IgG1-dependent toxicity mediated by a significant reduction of CD64+ cells after treatment. Analysing the multilineage differentiation potential of CD34+ cells, 40 and 200 ng/ml 20D9-ADC had no significant effect on the differentiation capacity compared to the untreated control (FIG. 5D). Treatment with 1 μg/ml 20D9-ADC revealed a significantly decreased proportion of hematopoietic stem cells (HSC), CD34+CD38−, CD34+CD38+, multi-lymphoid progenitor (MLP), common myeloid progenitor (CMP), and granulocyte-monocyte progenitor (GMP) cell populations. After treatment with IgG1-ADC, we observed significantly decreased MLPs, CMPs, and GMPs but to a lower extent compared to the 20D9 ADC. Furthermore, we assessed clonogenic capacity by colony forming unit (CFU) assay of healthy CD34+ cells after treatment (FIG. 5E). Whereas 40 ng/ml or 200 ng/ml 20D9-ADC showed no significant differences compared to the untreated control, treatment with 1 μg/ml 20D9-ADC and IgG1-ADC revealed significantly reduced granulocytic, monocytic and granulocytic-macrophagic colony formation. The erythroid progenitors were unaffected.

### Treatment Combination of 20D9-ADC and TKIs.

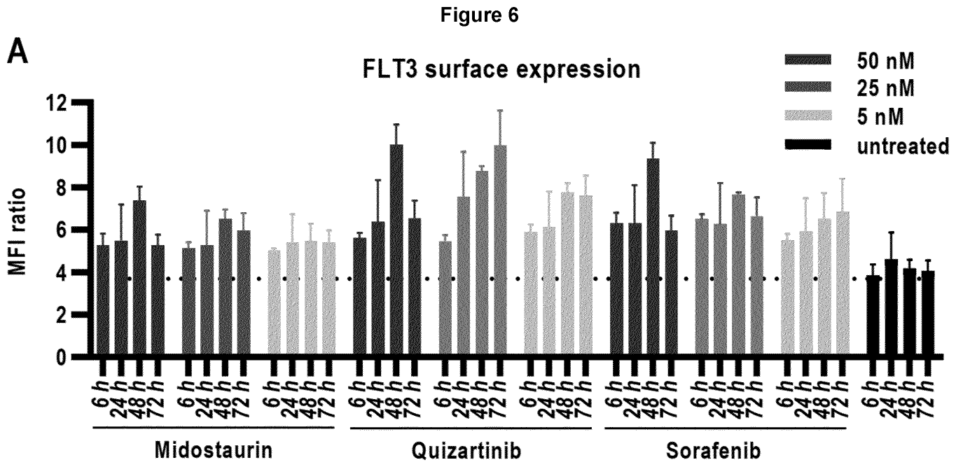

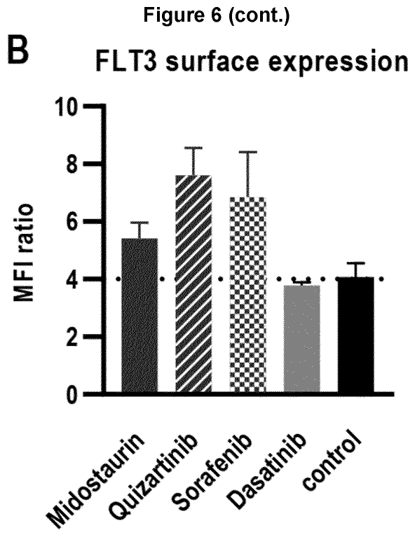

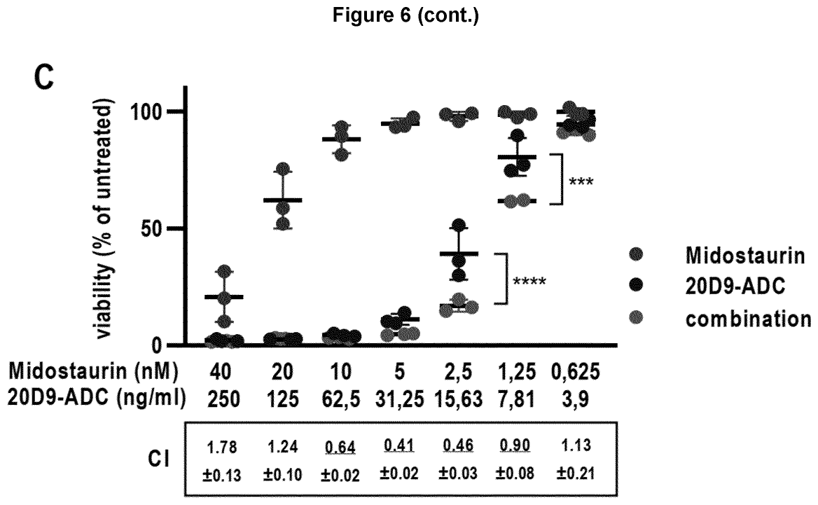

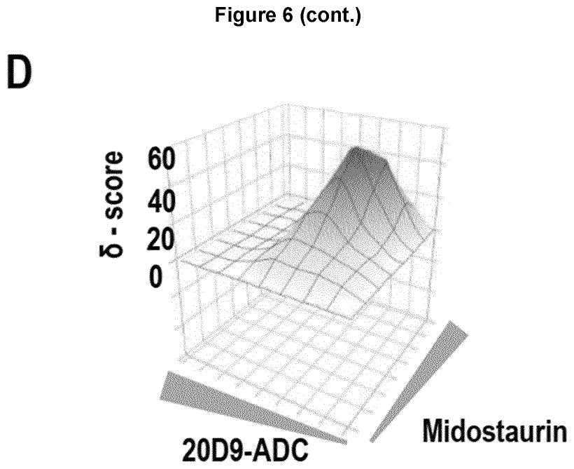

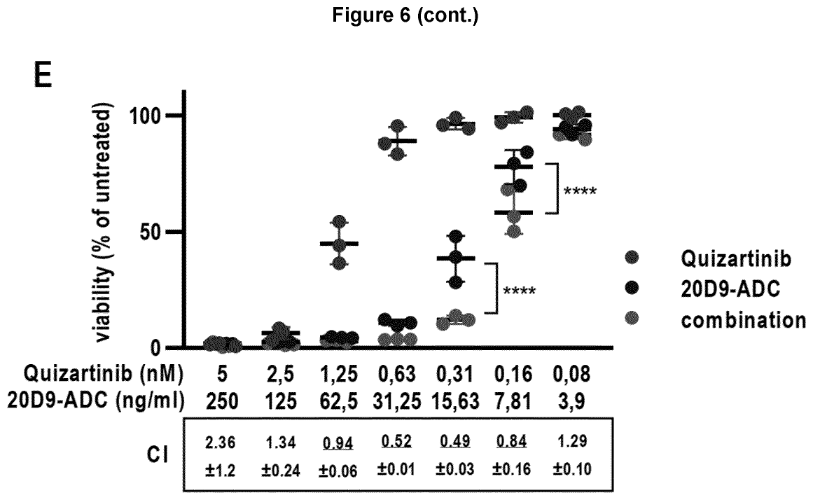

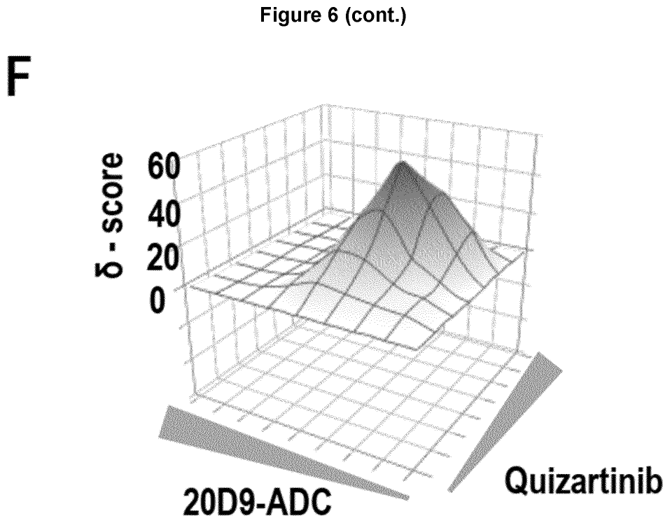

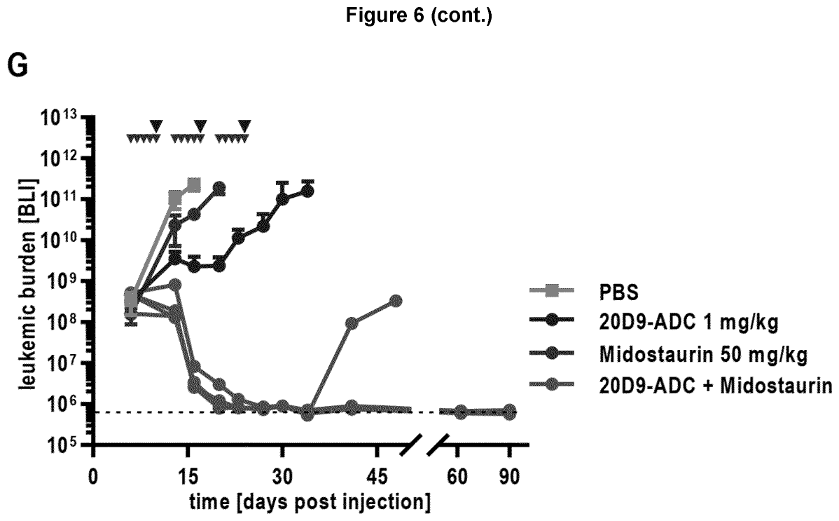

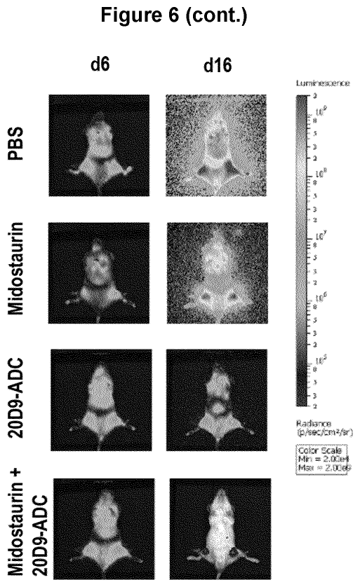

We combined the 20D9-ADC with Midostaurin, a multi-kinase inhibitor approved for treatment of FLT3-ITD+ AML, to increase the cytotoxic activity of the ADC. Incubation of MOLM-13 cell lines with different concentrations of the TKIs Midostaurin, Quizartinib (AC220) and Sorafenib led to increase of FLT3 cell surface expression after 6, 24, 48 and 72 h incubation (FIG. 6A,B). As control, Dasatinib, which does not affect cell viability of Molm13 cells even in high concentrations (data not shown) does not induce a FLT3 cell surface upregulation (FIG. 6B). Different dose combinations of 20D9-ADC and TKI were applied to MOLM-13 cells in vitro. While Midostaurin as single drug had no effect on cell viability at low doses, combination-treatment with the 20D9-ADC was significantly beneficial compared to 20D9-ADC treatment alone (FIG. 6C). Same effects could be observed for the combination of 20D9-ADC and AC220, another TKI (FIG. 6E). To calculate synergistic effects, we utilized different calculation methods like the Combination Index (CI)⁴⁰ or the ZIP method (Yadav B, Wennerberg K, Aittokallio T, Tang J. Comput Struct Biotechnol J. 2015; 13:504-513) using the Synergy Finder (lanevski A, Giri A K, Aittokallio T. Nucleic Acids Res. 2021; 48 (1): W488-W493). Thus, we could show a synergism for a combined treatment approach with 20D9-ADC and FLT3-TKI (FIG. 6C-F). In the MOLM-13 xenograft model described above, treatment with Midostaurin (50 mg/kg; Q5W×3; per os) or 20D9-ADC 1 mg/kg (Q1W×3; i.v.) as single agents only led to modest growth delay (FIG. 6G). Strikingly, combination of 20D9-ADC and midostaurin treatment led to drastic tumor reduction and probably cure in two out the three tumor-bearing animals. Thus, these results indicate a high synergistic potential of the FLT3 specific ADC when combined with FLT3 TKIs.

### Discussion

Here we report the development and preclinical characterization of a novel FLT3-targeting ADC, 20D9-ADC, which utilizes the recently developed P5 conjugation technology to conjugate MMAF, resulting in an ADC with robust preclinical activity in multiple models of AML. Further, we found a promising synergistic effect of the combination treatment of 20D9 ADC with a recently approved TKI in FLT3 mutated AML. FLT3 provides an excellent expression profile in AML and healthy non-hematopoietic tissues compared to other commonly discussed targets in AML treatment like CD33, CD123 and CLL1. The low RNA expression in lung, pancreas and brain was not yet confirmed to result in cell surface expression of FLT3. Thus, agents targeting FLT3 in AML may have the largest therapeutic index compared to other targets and is expected to have little to no healthy tissue toxicity outside of potential haematological toxicities. FLT3 is an established and popular target for TKIs, which can only be used in FLT3 mutated AMLs with permanently activated receptor signaling. However, FLT3 offers the advantage that it is overexpressed in the AML in comparison to healthy tissue, regardless of the mutation status or disease state. This is what makes FLT3 so interesting as an ADC target in AML treatment, as it allows a wide range of patients to be addressed. So far, Gemtuzumab ozogamicin (Mylotarg®) targeting CD33 is the only approved ADC in AML. The conjugation technology and linker design in ADC development is essential as they influence the toxicology profile. For example, the linker of Gemutzumab Ozogamicin exhibits instability, leading to premature release of calicheamycin. For 20D9-ADC development, the novel P5 conjugation technology based on Ethynylphosphonamidates with outstanding serum stability characteristics was applied to conjugate MMAF via a cleavable linker, which facilitates efficient intracellular release of the MMAF and is successfully used in approved ADCs. MMAF belongs, like MMAE, to the microtubule-targeting agents which are used as payloads in two-thirds of all clinical stage ADC. It is a highly potent agent with IC50 in the subnanomolare range and has lower bystander killing effects in comparison to MMAE, which is an advantage in haematological malignancies. We show here in vitro that the 20D9-ADC had strong and selective cytotoxicity in FLT3+ cell lines while we could clearly distinguish between the CD64 mediated and FLT3 mediated cytotoxicity in a cell line model and with deglycosylated ADCs in vitro, showing a strong advantage of co-targeting of both receptors. In vivo, we found a dose dependent response of aggressive AML cell lines to 20D9-ADC independent from the tumor burden at start of treatment, proofing the robustness and high efficacy of 20D9-ADC. Interestingly, the deglycosylated 20D9-ADC achieved almost the same efficacy compared to the native 20D9-ADC in MOLM-13 cells in vivo, despite targeting exclusively FLT3. Applying IgG1-ADC in vivo was much less effective than 20D9-ADC either deglycosylated or native, indicating that FLT3 targeting might be sufficient and superior compared to dual targeting or CD64 targeting in the NSG mice model. Moreover, we could also successfully treat AML patient derived xenograft models in vivo. These PDX samples recapitulate the phenotype of human AML since they comprise of AML stem cells and sub clonal AML cell populations. In the in vivo studies, the ADCs were well tolerated as single agent or in combination with tyrosine kinase inhibitors. To evaluate the toxicity profile of 20D9-ADC in healthy tissue, we focused on hematopoietic stem and progenitor cells, since the FLT3 expression in the brain, pancreas and lung tissue seems to be limited to the cytoplasm or to be very low. 20D9-ADC in concentrations in the range of IC₅₀ values of AML cell lines did not affect healthy human CD34+ cells, which is promising for a favourable toxicity profile. Only in high concentrations, the 20D9-ADC but also the IgG1-ADC shows cytotoxicity towards myelomonocytic and lymphoid progenitors. Thus, Fc receptor engagement might be a possible cause of side effects and toxicity toward megacaryocytes resulting in thrombocytopenia was reported. On the other hand, brentuximab-vedotin, an approved IgG1-based ADC in Hodgkin lymphoma, showed manageable tolerability and safety profile in a phase III study. Using Fc receptor interaction might also have advantages as it was reported that IgG1 can mediate antibody-dependent cell-mediated cytotoxicity (ADCC) and antibody-dependent cellular phagocytosis (ADCP) in the context of drug conjugates. Of note, our studies have shown a superior cytotoxic activity of the native 20D9-ADC compared to the deglycosylated 20D9ADC (that is devoid of FcR binding) in vitro, but not in vivo. It is unclear whether the FcR binding properties of the 20D9-ADC will be beneficial in AML patients with respect to toxicity and efficacy. Therefore, further studies in humans or nonhuman primates will be necessary to answer this question. Regarding FLT3 targeting the toxicology might be favourable, since a FLT3-CD3 bispecific antibody in cynomolgues monkey revealed a reversible depletion of dendritic cells, HSPCs and monocytes without any major clinical signs of toxicity. Due to the observed high efficacy, we evaluated the potential of 20D9-ADC for therapy of FLT3 mutated AML. By combining 20D9-ADC and FLT3 TKIs, we aimed at (1) exploiting the potential of the FLT3 target, (2) opening the therapeutic window for the FLT3 specific ADC treatment while reducing side effects and at (3) integrating an FLT3 ADC in the therapeutic landscape of AML. The combination of 20D9-ADC and TKI treatment showed significantly higher cytotoxicity in vitro compared to single drug treatment. The in vivo experiments resulted in even more striking benefit of the combination therapy of low dose 20D9-ADC and Midostaurin. We hypothesize that the outstanding treatment efficacy of the drug combination of 20D9-ADC and Midostaurin is due to an upregulation of the FLT3-ITD receptor on the cell surface as previous reported by our lab. However, we cannot exclude other mechanisms as Midostaurin is not specific for FLT3 and inhibits also other kinases like VEGFR-2, PDGFR and KIT. Similar to our results, Fu Li et al. described an anti CD123-ADC to be more efficient in combination with quizartinib. Further, a CD33 targeting ADC (IMGN779) showed increased effectivity in combination with quizartinib. In conclusion, we have developed and characterized a novel FLT3-targeting ADC that demonstrated potent antileukemic activity in preclinical models of AML including patient derived xenograft mouse models. Importantly, 20D9-ADC was effective at low concentrations in combination with midostaurin, suggesting a possible treatment concept with favourable toxicity profile. Our data indicate that FLT3 is a clinically promising target for ADC application which should be further evaluated in clinical studies in combination with FLT3 inhibitors.

### Example 2: Conjugation of P5 (OEt)-VC-PAB-MMAF to the Anti-FLT-3 Antibodies as Used Herein

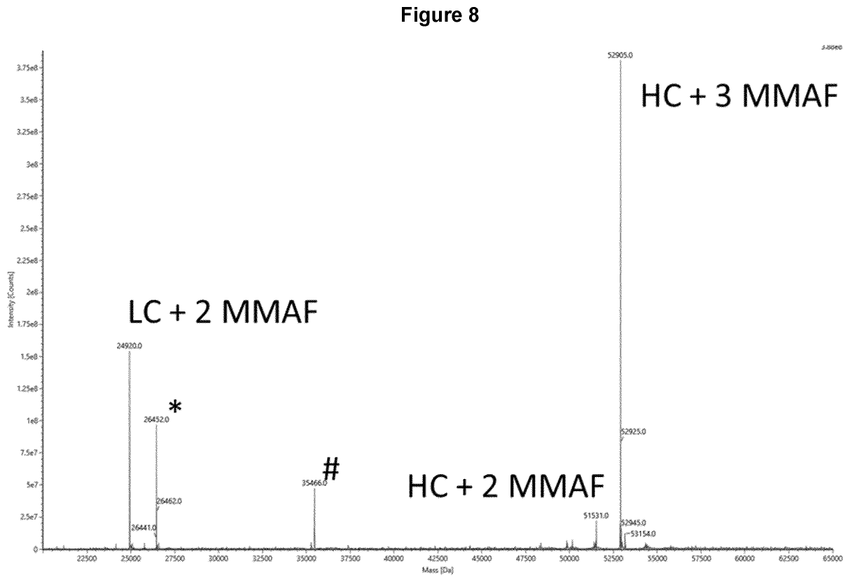

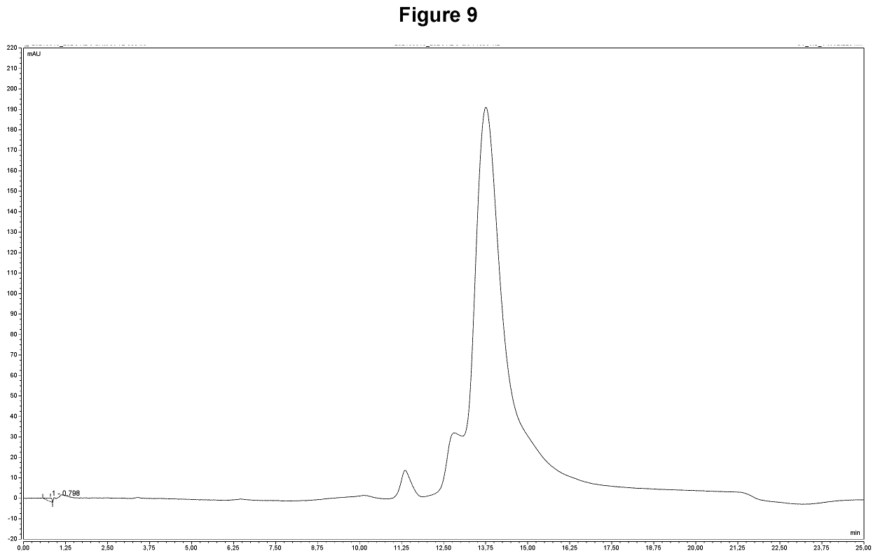

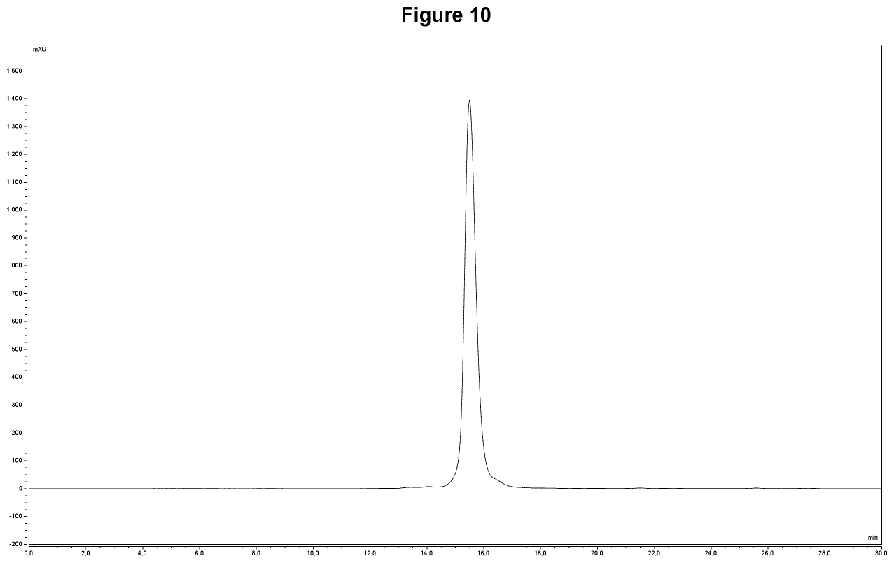

P5 (OEt)-VC-PAB-MMAF has been synthesized as previously reported (Kasper et al., 2019: Ethynylphosphonamidates for the Rapid and Cysteine-Selective Generation of Efficacious Antibody-Drug Conjugates; https://doi.org/10.1002/anie.201904193). The anti-FLT3 antibodies have been concentrated to 10.0 mg/mL in a buffer, containing 100 mM NH₄HCO₃ pH 8.3, by using Vivaspin centrifugal concentrators (Sartorius, Germany) with a MWCO of 30 kDa. 700 μL of this solution have been mixed with 37.3 μL of a 10 mM solution of TCEP in the same buffer. TCEP (Merck KGaA, Germany) has been purchased as 0.5 Mol/L neutral solution. Directly after TCEP addition, 23.3 μL of a solution containing 40 mM of 1 (P5 (OEt)-VC-PAB-MMAF) in DMSO (Merck KGaA, Germany) have been added and the clear solution has been gently shaken for 16 h at 25° C. The crude solution has ben purified by size-exclusion chromatography, eluting with sterile PBS (Merck KGaA, Germany). ADC containing fractions have been pooled. The final concentration was determined in a 96-well plate with a Pierce™ Rapid Gold BCA Protein Assay Kit (Thermo Fisher Scientific, USA) and a Bradford reagent B6916 (Merck, Germany) with pre-diluted protein assay standards of bovine gamma globulin (Thermo Fisher Scientific, USA). Results of both Assays were arithmetically averaged. Afterwards, the ADCs were analyzed via LC/MS, HIC- and SEC-HPLC. The results are shown in FIGS. 8, 9, 10 are exemplaric datasets that have been recorded with the 20D9 antibody clone. Other mabs described hereien behaved very similar in the conjugation reaction and let to similar analytical results as depicted in FIGS. 8, 9 and 10. ADC analysis was carried out according to the following methods:

### Preparative Size-Exclusion Chromatography

Preparative size-exclusion chromatography Protein purification by size-exclusion chromatography was conducted with an ÄKTA FPLC system (GE Healthcare, United States) equipped with a P-920 pump system, a UPC-900 detector and a FRAC-950 fraction collector.

### Analytical SEC- and HIC-HPLC

Analytical size-exclusion chromatography (A-SEC) of the ADCs was conducted on a Vanquish Flex UHPLC System with a DAD detector, Split Sampler FT (4° C.), Column Compartment H (25° C.) and binary pump F (Thermo Fisher Scientific, USA) using a MAbPac SEC-1 300 Å, 4×300 mm column (Thermo Fisher Scientific, USA) with a flow rate of 0.15 mL/min. Separation of different ADC/mAb populations have been achieved during a 30 minute isocratic gradient using a phosphate buffer at pH 7 (20 mM Na2HPO4/NaH2PO4, 300 mM NaCl, 5% v/v isopropyl alcohol as a mobile phase. 8 μg ADC/mAb where loaded onto the column for A-SEC analysis.

UV chromatograms were recorded at 220 and 280 nm. Quantification of monomer and HMWS was achieved after integration of the peak area at 220 nm.

### MS-Analysis

ADCs were deglycosylated and reduced prior MS analysis. 50 μL of a 0.2 mg/mL solution of the ADC were mixed with 0.5 μl PNGase-F solution (Pomega, Germany, Recombinant, cloned from Elizabethkingia miricola 10 u/μl) and 5 μL of a solution of 10 mM DTT (Merck, Germany) in PBS. The mixture was incubated at 37° C. for at least 2 hours before MS analysis. 2 μL were injected per analysis.

Reduced and deglycosylated ADCs were analyzed using a Waters H-class instrument equipped with a quaternary solvent manager, a Waters sample manager-FTN, a Waters PDA detector and a Waters column manager with an Acquity UPLC protein BEH C4 column (300 Å, 1.7 μm, 2.1 mm×50 mm). Proteins were eluted with a flow rate of 0.3 mL/min. The following gradient was used: A: 0.01% FA in H2O; B: 0.01% FA in MeCN. 5-95% B 0-6 min. Mass analysis was conducted with a Waters XEVO G2-XS QTof analyzer. Proteins were ionized in positive ion mode applying a cone voltage of 40 kV. Raw data was analyzed with MaxEnt 1 and deconvoluted until convergent.

### Example 3: Binding and Cytotoxicity of Exemplary ADCs of the Present Invention

### Methods:

### Cell Lines

Cell lines were cultured according to the supplier's recommendations. For stable recombinant protein expression, Ba/F3 cells were retrovirally transduced as described before (e.g., Polzer H, Janke H, Schmid D, Hiddemann W, Spiekermann K. Casitas B-lineage lymphoma mutants activate AKT to induce transformation in cooperation with class III receptor tyrosine kinases. Exp Hematol. 2013; 41 (3): 271-280.e4. doi: 10.1016/j.exphem.2012.10.016).

### DNA Constructs Expressed in Ba/F3 Cells

Murine FLT3 (OMu21985D, Genescript), human CD64 (RC207487), CD32 (RC205786) and CD16 (RC206429), were purchased from Origene. Cynomolgues monkey FLT3 gene was synthesized from XM_015439107.1 by Eurofins. The constructs were cloned into retroviral expression vector MSCV-IRES-YFP (pMIY) using the In-Fusion HD Cloning Plus Kit (Takara Bio, Saint-Germain-en-Laye, France). All FLT3 point mutations were generated using the QuikChange II XL Site-Directed Mutagenesis Kit (Agilent Technologies, Santa Clara, CA, USA) and correct sequence was confirmed by Sanger sequencing. MSCV-IRES-YFP vectors with inserted human wildtype FLT3 and ITD (w51 and NPOS) mutated FLT3 has been described before (e.g., Opatz S, Polzer H, Herold T, et al. Exome sequencing identifies recurring FLT3 N676K mutations in core-binding factor leukemia. Blood. 2013; 122 (10): 1761-1769. doi: 10.1182/blood-2013-01-476473).

### p53 Knock Down in AML Cell Lines

### Protein Expression and Binding of Monoclonal Antibodies

For FLT3 expression analysis, cells were stained on ice with primary mouse anti-FLT3-AF647 antibodies for 30 min. For antibody binding studies, cells were stained on ice with chimeric (20D9) or humanized anti-FLT3 antibodies (in house) or IgG1 control with or without Leu234Ala/Leu235Ala (LALA)-mutation in the Fc region and secondary antibody goat F(ab′)2 Anti-Human IgG-AF647 (2042-31) purchased from Southern Biotech for 30 min, respectively. Binding and expression were subsequently measured at the flow cytometer (BD FACSCanto II) and the results were evaluated with FlowJo version 10.8.1.

### Enzyme-Linked Immunosorbent Assay (ELISA)

Maxisorp plates were coated with human recombinant FLT3 protein (10445-H08H, Sino Biological). Chimeric (20D9) or humanized anti-FLT3 monoclonal antibodies (in house) and secondary alkaline phosphatase conjugated rabbit anti-human IgG antibody (309-055-008, Jackson Immunoresearch) were used. For detection, Attophos Fluorescent Substrate System (S1000, Promega) was applied. The fluorescence was read with excitation of 405 nm and emission at 500-550 nm. The calculation of the dissociation constant KD was performed using GraphPad Prism version 9.4.0.

### Cytotoxicity Proliferation Assays

Suspension cells were treated with 20D9 ADC or #3 ADC (in-house). AML cells were treated once (d 0) and viability was determined after 96 h using resazurin solution (50 μM final concentration, 4 h incubation) (R12204, Thermo Fisher Scientific). For Ba/F3 cell assays, cells were treated once (d 0) and viable cells were counted after 72 h on Vi Cell Cell Viability Analyzer (Beckman Coulter, Krefeld, Germany). Calculation of IC50 values was performed using GraphPad Prism version 9.4.0 (GraphPad Software, La Jolla, CA, USA).

### Results:

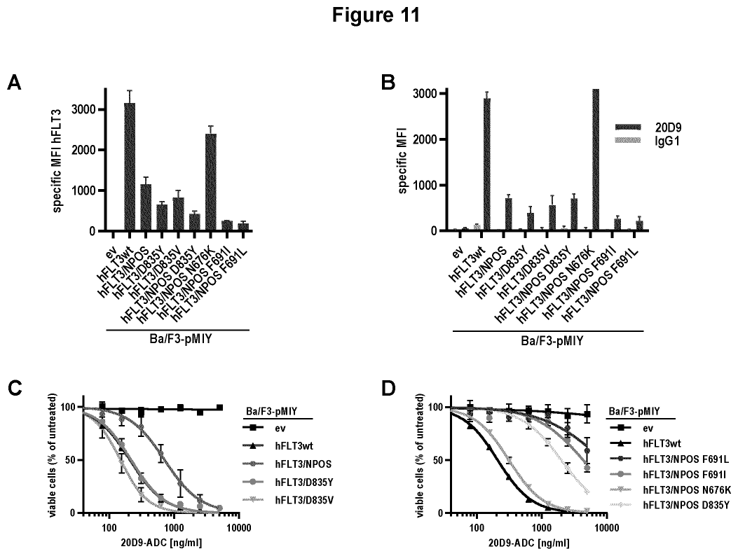

In FIG. 11, it is shown that the chimeric clone 20D9 also binds to FLT3 with common mutations, which provides an advantage over FLT3-antibodies known in the art. Accordingly, next to FLT3 wt, 20D9-ADC also binds to and acts on FLT3ITD and FLT3TKD mutants. A smaller binding and cytotoxic effect correlate with lower expression of the FLT3 variant.

Evaluation of binding and cytotoxicity of 20D9-ADC on FLT3ITD and TKD mutants was carried out as shown in FIG. 11, according to which (A,B) Cell surface expression of FLT3 receptor (A) or cell surface binding of 20D9 mab and control hIgG1 antibody (B) in Ba/F3 cells stably expressing pMIY (Ba/F3-pMIY) empty vector (ev), wildtype (hFLT3wt) or FLT3 mutants FLT3/NPOS, FLT3/D835Y, FLT3/D835V, FLT3/NPOS D835Y, FLT3/NPOS N676K, FLT3/NPOS F6911 or FLT3/NPOS F691L was measured in flow cytometry; Mean±s.d. of n=3; (C,D) Treatment of Ba/F3 cells stably expressing pMIY (Ba/F3-PMIY) ev, FLT3 wt, FLT3 mutants FLT3/NPOS, FLT3/D835Y, FLT3/D835V (C) or FLT3/NPOS D835Y, FLT3/NPOS N676K, FLT3/NPOS F6911 FLT3/NPOS F691L (D) with different concentrations of 20D9-ADC; Viability was determined after 72h by trypan blue exclusion count and compared to untreated control; Mean±s.d. of n=3 biological replicates.

Further, it was shown that a TP53 knockdown in FLT3 positive AML cell lines only slightly altered IC50 of 20D9-ADC, indicating that a TP53 mutation is not likely to compromise efficacy. Accordingly, since TP53 is a common mutation in AML, it is shown here that it hardly affects the binding of the chimeric 20D9 clone.

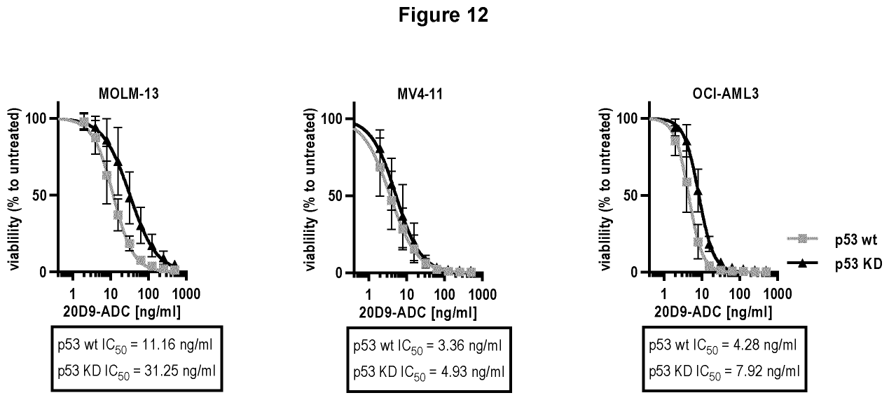

Cytotoxicity of 20D9-ADC on p53 wt and KD cell lines was carried out as shown in FIG. 12, according to which cytotoxicity of 20D9-ADC in native or p53 KD MV4-11, MOLM-13 or OCI-AML 3 cells was evaluated; Viability was determined after 96h treatment with different concentrations by resazurin fluorescence and normalized to untreated control; Mean±s.d. of n=3 biological replicates.

In the next step, the variable regions of the FLT3 20D9 antibody have been humanized with the help of bioinformatic models. For this, the sequence of the chimeric 20D9 antibody was analyzed and appropriate human acceptor frameworks (VH and VL separately) were identified. Amino acid differences between the murine and human frameworks were identified in silico, ranked with respect to biochemical similarity and thereby, the different VH and VL sequences were designed. During this process, four candidate sequences for the light and heavy chains have been generated, respectively. These sequences have been combined in a 4×4 matrix to yield 16 different antibody candidates with a degree of humanization between 90.2% and 100% (versus 84.7% in the original 20D9 clone). These candidates were transiently expressed in ExpiCHO cells. Four of the 16 candidates were excluded from further analysis due to insufficient production efficiency. The remaining 12 candidates were subjected to stability analysis at three different temperatures. In regular intervals, samples were analysed by UV-Vis spectrometry, SDS-PAGE, size exclusion chromatography (SEC) and hydrophobic interaction chromatography (HIC) for the presence of aggregates and impurities. All 12 antibodies were stable in the course of the analyses and did not show significant aggregation in the above-mentioned assays:

Humanized FLT3 antibodies (CDRs were
determined following Kabat):
Clone #1 (VH1 + VK1):
Clone #1 Heavy Chain
Sequence without signal peptide
CDRs
VH (Heavy chain variable sequence)
HC (Heavy chain constant sequence)
SEQ ID NO: 63:
QVMLVESGGGVVQPGRSLRLSCAASGFTFNNYWMTWVRQAPGKGL
EWIASITKTGGGTYYPDSVKGRFTISRDNAKSTLYLQMNSLRSED
TATYYCTTLQQLGVMDAWGQGASVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 64:
QVMLVESGGGVVQPGRSLRLSCAASGFTFNNYWMTWVRQAPGKGL
EWIASITKTGGGTYYPDSVKGRFTISRDNAKSTLYLQMNSLRSED
TATYYCTTLQQLGVMDAWGQGASVTVSS
SEQ ID NO: 65: CDR-H1: NYWMT
SEQ ID NO: 66: CDR-H2: SITKTGGGTYYPDSVKG
SEQ ID NO: 67: CDR-H3: LQQLGVMDA
Clone #1 Light Chain
(Changes in CDRs are highlighted
in size, Changes in the framework
are not highlighted)
Sequence without signal peptide
CDRs
VL (Light chain variable sequence)
LC (Light chain constant sequence)
SEQ ID NO: 68
DIQMTQSPSVLSASVGDRVTINC [symbol] WYQQKLGEAPK
LLIY [symbol] GVPSRFSGSGSGTDYTLTISSLQPEDVATYFCFQ
HKSWPLTFGSGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 69
DIQMTQSPSVLSASVGDRVTINC [symbol] WYQQKLGEAPK
LLIY [symbol] GVPSRFSGSGSGTDYTLTISSLQPEDVATYFCF
QHKSWPLTFGSGTKLEIK
SEQ ID NO: 70 CDR-L1: RASQNINKELN
SEQ ID NO: 71 CDR-L2: NTNNLQS
SEQ ID NO: 72 CDR-L3: FQHKSWPLT
Clone #2 (VH2 + VK1)
Clone #2 Heavy Chain
Sequence without signal peptide
CDRs
VH (Heavy chain variable sequence)
HC (Heavy chain constant sequence)
SEQ ID NO: 73
QVMLVESGGGVVQPGRSLRLSCAASGFTFNNYWMTWVRQAPGKGL
EWIASITKTGGGTYYPDSVKGRFTISRDNSKSTLYLQMNSLRAED
TATYYCTTLQQLGVMDAWGQGASVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 74
QVMLVESGGGVVQPGRSLRLSCAASGFTFNNYWMTWVRQAPGKGL
EWIASITKTGGGTYYPDSVKGRFTISRDNSKSTLYLQMNSLRAED
TATYYCTTLQQLGVMDAWGQGASVTVSS
SEQ ID NO: 75 CDR-H1: NYWMT
SEQ ID NO: 76 CDR-H2: SITKTGGGTYYPDSVKG
SEQ ID NO: 77 CDR-H3: LQQLGVMDA
Clone #2 Light Chain
Sequence without signal peptide
CDRs
VL (Light chain variable sequence)
LC (Light chain constant sequence)
SEQ ID NO: 78
DIQMTQSPSVLSASVGDRVTINCRASQNINKELNWYQQKLGEAPK
LLIYNTNNLQSGVPSRFSGSGSGTDYTLTISSLQPEDVATYFCFQ
HKSWPLTFGSGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 79
DIQMTQSPSVLSASVGDRVTINCRASQNINKELNWYQQKLGEAPK
LLIYNTNNLQSGVPSRFSGSGSGTDYTLTISSLQPEDVATYFCFQ
HKSWPLTFGSGTKLEIK
SEQ ID NO: 80 CDR-L1: RASQNINKELN
SEQ ID NO: 81 CDR-L2: NTNNLQS
SEQ ID NO: 82 CDR-L3: FQHKSWPLT
Clone #3 (VH3 + VK1), preferred clone/embodiment
Clone #3 Heavy Chain
Sequence without signal peptide
CDRs
VH (Heavy chain variable sequence)
HC (Heavy chain constant sequence)
SEQ ID NO: 83
QVQLVESGGGVVQPGRSLRLSCAASGFTFNNYWMTWVRQAPGKGL
EWIASITKTGGGTYYADSVKGRFTISRDNSKNTLYLQMNSLRAED
TAVYYCTTLQQLGVMDAWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 84
QVQLVESGGGVVQPGRSLRLSCAASGFTFNNYWMTWVRQAPGKGL
EWIASITKTGGGTYYADSVKGRFTISRDNSKNTLYLQMNSLRAED
TAVYYCTTLQQLGVMDAWGQGTLVTVSS
SEQ ID NO: 85 CDR-H1: NYWMT
SEQ ID NO: 86 CDR-H2: SITKTGGGTYYADSVKG
SEQ ID NO: 87 CDR-H3: LQQLGVMDA
Clone #3 Light Chain
Sequence without signal peptide
CDRs
VL (Light chain variable sequence)
LC (Light chain constant sequence)
SEQ ID NO: 88
DIQMTQSPSVLSASVGDRVTINCRASQNINKELNWYQQKLGEAPK
LLIYNTNNLQSGVPSRFSGSGSGTDYTLTISSLQPEDVATYFCFQ
HKSWPLTFGSGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 89
DIQMTQSPSVLSASVGDRVTINCRASQNINKELNWYQQKLGEAPK
LLIYNTNNLQSGVPSRFSGSGSGTDYTLTISSLQPEDVATYFCFQ
HKSWPLTFGSGTKLEIK
SEQ ID NO: 90 CDR-L1: RASQNINKELN
SEQ ID NO: 91 CDR-L2: NTNNLQS
SEQ ID NO: 92 CDR-L3: FQHKSWPLT
Clone #4 (VH4 + VK1)
Clone #4 Heavy Chain
Sequence without signal peptide
CDRs
VH (Heavy chain variable sequence)
HC (Heavy chain constant sequence)
SEQ ID NO: 93
QVQLVESGGGVVQPGRSLRLSCAASGFTFNNYWMTWIRQAPGKGL
EWIASITKTGGGTYYADSVKGRFTISRDNSKNTLYLQMNSLRAED
TAVYYCTTLQQLGVMDAWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 94
QVQLVESGGGVVQPGRSLRLSCAASGFTFNNYWMTWIRQAPGKGL
EWIASITKTGGGTYYADSVKGRFTISRDNSKNTLYLQMNSLRAED
TAVYYCTTLQQLGVMDAWGQGTLVTVSS
SEQ ID NO: 95 CDR-H1: NYWMT
SEQ ID NO: 96 CDR-H2: SITKTGGGTYYADSVKG
SEQ ID NO: 97 CDR-H3: LQQLGVMDA
Clone #4 Light Chain
Sequence without signal peptide
CDRs
VL (Light chain variable sequence)
LC (Light chain constant sequence)
SEQ ID NO: 98
DIQMTQSPSVLSASVGDRVTINCRASQNINKELNWYQQKLGEAPK
LLIYNTNNLQSGVPSRFSGSGSGTDYTLTISSLQPEDVATYFCFQ
HKSWPLTFGSGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 99
DIQMTQSPSVLSASVGDRVTINCRASQNINKELNWYQQKLGEAPK
LLIYNTNNLQSGVPSRFSGSGSGTDYTLTISSLQPEDVATYFCFQ
HKSWPLTFGSGTKLEIK
SEQ ID NO: 100 CDR-L1: RASQNINKELN
SEQ ID NO: 101 CDR-L2: NTNNLQS
SEQ ID NO: 102 CDR-L3: FQHKSWPLT
Clone #5 (VH1 + VK2)
Clone #5 Heavy Chain
Sequence without signal peptide
CDRs
VH (Heavy chain variable sequence)
HC (Heavy chain constant sequence)
SEQ ID NO: 103
QVMLVESGGGVVQPGRSLRLSCAASGFTFNNYWMTWVRQAPGKGL
EWIASITKTGGGTYYPDSVKGRFTISRDNAKSTLYLQMNSLRSED
TATYYCTTLQQLGVMDAWGQGASVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 104
QVMLVESGGGVVQPGRSLRLSCAASGFTFNNYWMTWVRQAPGKGL
EWIASITKTGGGTYYPDSVKGRFTISRDNAKSTLYLQMNSLRSED
TATYYCTTLQQLGVMDAWGQGASVTVSS
SEQ ID NO: 105 CDR-H1: NYWMT
SEQ ID NO: 106 CDR-H2: SITKTGGGTYYPDSVKG
SEQ ID NO: 107 CDR-H3: LQQLGVMDA
Clone #5 Light Chain
Sequence without signal peptide
CDRs
VL (Light chain variable sequence)
LC (Light chain constant sequence)
SEQ ID NO: 108:
DIQMTQSPSVLSASVGDRVTITCRASQNINKELNWYQQKLGKAPK
LLIYNTNNLQSGVPSRFSGSGSGTDFTLTISSLQPEDFATYYCFQ
HKSWPLTFGSGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 109
DIQMTQSPSVLSASVGDRVTITCRASQNINKELNWYQQKLGKAPK
LLIYNTNNLQSGVPSRFSGSGSGTDFTLTISSLQPEDFATYYCFQ
HKSWPLTFGSGTKLEIK
SEQ ID NO: 110 CDR-L1: RASQNINKELN
SEQ ID NO: 111 CDR-L2: NTNNLQS
SEQ ID NO: 112 CDR-L3: FQHKSWPLT
Clone #6 (VH2 + VK2)
Clone #6 Heavy Chain
Sequence without signal peptide
CDRs
VH (Heavy chain variable sequence)
HC (Heavy chain constant sequence)
SEQ ID NO: 113
QVMLVESGGGVVQPGRSLRLSCAASGFTFNNYWMTWVRQAPGKGL
EWIASITKTGGGTYYPDSVKGRFTISRDNSKSTLYLQMNSLRAED
TATYYCTTLQQLGVMDAWGQGASVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 114
QVMLVESGGGVVQPGRSLRLSCAASGFTFNNYWMTWVRQAPGKGL
EWIASITKTGGGTYYPDSVKGRFTISRDNSKSTLYLQMNSLRAED
TATYYCTTLQQLGVMDAWGQGASVTVSS
SEQ ID NO: 115 CDR-H1: NYWMT
SEQ ID NO: 116 CDR-H2: SITKTGGGTYYPDSVKG
SEQ ID NO: 117 CDR-H3: LQQLGVMDA
Clone #6 Light Chain
Sequence without signal peptide
CDRs
VL (Light chain variable sequence)
LC (Light chain constant sequence)
SEQ ID NO: 118
DIQMTQSPSVLSASVGDRVTITCRASQNINKELNWYQQKLGKAPK
LLIYNTNNLQSGVPSRFSGSGSGTDFTLTISSLQPEDFATYYCFQ
HKSWPLTFGSGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 119
DIQMTQSPSVLSASVGDRVTITCRASQNINKELNWYQQKLGKAPK
LLIYNTNNLQSGVPSRFSGSGSGTDFTLTISSLQPEDFATYYCFQ
HKSWPLTFGSGTKLEIK
SEQ ID NO: 120 CDR-L1: RASQNINKELN
SEQ ID NO: 121 CDR-L2: NTNNLQS
SEQ ID NO: 122 CDR-L3: FQHKSWPLT
Clone #7 (VH3 + VK29)
Clone #7 Heavy Chain
Sequence without signal peptide
CDRs
VH (Heavy chain variable sequence)
HC (Heavy chain constant sequence)
SEQ ID NO: 123
QVQLVESGGGVVQPGRSLRLSCAASGFTFNNYWMTWVRQAPGKGL
EWIASITKTGGGTYYADSVKGRFTISRDNSKNTLYLQMNSLRAED
TAVYYCTTLQQLGVMDAWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 124
QVQLVESGGGVVQPGRSLRLSCAASGFTFNNYWMTWVRQAPGKGL
EWIASITKTGGGTYYADSVKGRFTISRDNSKNTLYLQMNSLRAED
TAVYYCTTLQQLGVMDAWGQGTLVTVSS
SEQ ID NO: 125 CDR-H1: NYWMT
SEQ ID NO: 126 CDR-H2: SITKTGGGTYYADSVKG
SEQ ID NO: 127 CDR-H3: LQQLGVMDA
Clone #7 Light Chain
Sequence without signal peptide
CDRs
VL (Light chain variable sequence)
LC (Light chain constant sequence)
SEQ ID NO: 128
DIQMTQSPSVLSASVGDRVTITCRASQNINKELNWYQQKLGKAPK
LLIYNTNNLQSGVPSRFSGSGSGTDFTLTISSLQPEDFATYYCFQ
HKSWPLTFGSGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 129
DIQMTQSPSVLSASVGDRVTITCRASQNINKELNWYQQKLGKAPK
LLIYNTNNLQSGVPSRFSGSGSGTDFTLTISSLQPEDFATYYCFQ
HKSWPLTFGSGTKLEIK
SEQ ID NO: 130 CDR-L1: RASQNINKELN
SEQ ID NO: 131 CDR-L2: NTNNLQS
SEQ ID NO: 132 CDR-L3: FQHKSWPLT
Clone #8 (VH4 + VK2)
Clone #8 Heavy Chain
Sequence without signal peptide
CDRs
VH (Heavy chain variable sequence)
HC (Heavy chain constant sequence)
SEQ ID NO: 133
QVQLVESGGGVVQPGRSLRLSCAASGFTFNNYWMTWIRQAPGKGL
EWIASITKTGGGTYYADSVKGRFTISRDNSKNTLYLQMNSLRAED
TAVYYCTTLQQLGVMDAWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 134
QVQLVESGGGVVQPGRSLRLSCAASGFTFNNYWMTWIRQAPGKGL
EWIASITKTGGGTYYADSVKGRFTISRDNSKNTLYLQMNSLRAED
TAVYYCTTLQQLGVMDAWGQGTLVTVSS
SEQ ID NO: 135 CDR-H1: NYWMT
SEQ ID NO: 136 CDR-H2: SITKTGGGTYYADSVKG
SEQ ID NO: 137 CDR-H3: LQQLGVMDA
Clone #8 Light Chain
Sequence without signal peptide
CDRs
VL (Light chain variable sequence)
LC (Light chain constant sequence)
SEQ ID NO: 138
DIQMTQSPSVLSASVGDRVTITCRASQNINKELNWYQQKLGKAPK
LLIYNTNNLQSGVPSRFSGSGSGTDFTLTISSLQPEDFATYYCFQ
HKSWPLTFGSGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 139
DIQMTQSPSVLSASVGDRVTITCRASQNINKELNWYQQKLGKAPK
LLIYNTNNLQSGVPSRFSGSGSGTDFTLTISSLQPEDFATYYCFQ
HKSWPLTFGSGTKLEIK
SEQ ID NO: 140 CDR-L1: RASQNINKELN
SEQ ID NO: 141 CDR-L2: NTNNLQS
SEQ ID NO: 142 CDR-L3: FQHKSWPLT
Clone #9 (VH1 + VK3)
Clone #9 Heavy Chain
Sequence without signal peptide
CDRs
VH (Heavy chain variable sequence)
HC (Heavy chain constant sequence)
SEQ ID NO: 143
QVMLVESGGGVVQPGRSLRLSCAASGFTFNNYWMTWVRQAPGKGL
EWIASITKTGGGTYYPDSVKGRFTISRDNAKSTLYLQMNSLRSED
TATYYCTTLQQLGVMDAWGQGASVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 144
QVMLVESGGGVVQPGRSLRLSCAASGFTFNNYWMTWVRQAPGKGL
EWIASITKTGGGTYYPDSVKGRFTISRDNAKSTLYLQMNSLRSED
TATYYCTTLQQLGVMDAWGQGASVTVSS
SEQ ID NO: 145 CDR-H1: NYWMT
SEQ ID NO: 146 CDR-H2: SITKTGGGTYYPDSVKG
SEQ ID NO: 147 CDR-H3: LQQLGVMDA
Clone #9 Light Chain
Sequence without signal peptide
CDRs
VL (Light chain variable sequence)
LC (Light chain constant sequence)
SEQ ID NO: 148
DIQMTQSPSSLSASVGDRVTITCRASQSINKELNWYQQKPGKAPK
LLIYNTNNLQSGVPSRFSGSGSGTDFTLTISSLQPEDFATYYCFQ
HKSWPLTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 149
DIQMTQSPSSLSASVGDRVTITCRASQSINKELNWYQQKPGKAPK
LLIYNTNNLQSGVPSRFSGSGSGTDFTLTISSLQPEDFATYYCFQ
HKSWPLTFGQGTKLEIK
SEQ ID NO: 150 CDR-L1: RASQSINKELN
SEQ ID NO: 151 CDR-L2: NTNNLQS
SEQ ID NO: 152 CDR-L3: FQHKSWPLT
Clone #10 (VH2 + VK3)
Clone #10 Heavy Chain
Sequence without signal peptide
CDRs
VH (Heavy chain variable sequence)
HC (Heavy chain constant sequence)
SEQ ID NO: 153
QVMLVESGGGVVQPGRSLRLSCAASGFTFNNYWMTWVRQAPGKGL
EWIASITKTGGGTYYPDSVKGRFTISRDNSKSTLYLQMNSLRAED
TATYYCTTLQQLGVMDAWGQGASVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 154
QVMLVESGGGVVQPGRSLRLSCAASGFTFNNYWMTWVRQAPGKGL
EWIASITKTGGGTYYPDSVKGRFTISRDNSKSTLYLQMNSLRAED
TATYYCTTLQQLGVMDAWGQGASVTVSS
SEQ ID NO: 155 CDR-H1: NYWMT
SEQ ID NO: 156 CDR-H2: SITKTGGGTYYPDSVKG
SEQ ID NO: 157 CDR-H3: LQQLGVMDA
Clone #10 Light Chain
Sequence without signal peptide
CDRs
VL (Light chain variable sequence)
LC (Light chain constant sequence)
SEQ ID NO: 158
DIQMTQSPSSLSASVGDRVTITCRASQSINKELNWYQQKPGKAPK
LLIYNTNNLQSGVPSRFSGSGSGTDFTLTISSLQPEDFATYYCFQ
HKSWPLTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 159
DIQMTQSPSSLSASVGDRVTITCRASQSINKELNWYQQKPGKAPK
LLIYNTNNLQSGVPSRFSGSGSGTDFTLTISSLQPEDFATYYCFQ
HKSWPLTFGQGTKLEIK
SEQ ID NO: 160 CDR-L1: RASQSINKELN
SEQ ID NO: 161 CDR-L2: NTNNLQS
SEQ ID NO: 162 CDR-L3: FQHKSWPLT
Clone #11 (VH3 + VK3)
Clone #11 Heavy Chain
Sequence without signal peptide
CDRs
VH (Heavy chain variable sequence)
HC (Heavy chain constant sequence)
SEQ ID NO: 163
QVQLVESGGGVVQPGRSLRLSCAASGFTFNNYWMTWVRQAPGKGL
EWIASITKTGGGTYYADSVKGRFTISRDNSKNTLYLQMNSLRAED
TAVYYCTTLQQLGVMDAWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 164
QVQLVESGGGVVQPGRSLRLSCAASGFTFNNYWMTWVRQAPGKGL
EWIASITKTGGGTYYADSVKGRFTISRDNSKNTLYLQMNSLRAED
TAVYYCTTLQQLGVMDAWGQGTLVTVSS
SEQ ID NO: 165 CDR-H1: NYWMT
SEQ ID NO: 166 CDR-H2: SITKTGGGTYYADSVKG
SEQ ID NO: 167 CDR-H3: LQQLGVMDA
Clone #11 Light Chain
Sequence without signal peptide
CDRs
VL (Light chain variable sequence)
LC (Light chain constant sequence)
SEQ ID NO: 168
DIQMTQSPSSLSASVGDRVTITCRASQSINKELNWYQQKPGKAPK
LLIYNTNNLQSGVPSRFSGSGSGTDFTLTISSLQPEDFATYYCFQ
HKSWPLTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 169
DIQMTQSPSSLSASVGDRVTITCRASQSINKELNWYQQKPGKAPK
LLIYNTNNLQSGVPSRFSGSGSGTDFTLTISSLQPEDFATYYCFQ
HKSWPLTFGQGTKLEIK
SEQ ID NO: 170 CDR-L1: RASQSINKELN
SEQ ID NO: 171 CDR-L2: NTNNLQS
SEQ ID NO: 172 CDR-L3: FQHKSWPLT
Clone #12 (VH4 + VK3)
Clone #12 Heavy Chain
Sequence without signal peptide
CDRs
VH (Heavy chain variable sequence)
HC (Heavy chain constant sequence)
SEQ ID NO: 173
QVQLVESGGGVVQPGRSLRLSCAASGFTFNNYWMTWIRQAPGKGL
EWIASITKTGGGTYYADSVKGRFTISRDNSKNTLYLQMNSLRAED
TAVYYCTTLQQLGVMDAWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 174
QVQLVESGGGVVQPGRSLRLSCAASGFTFNNYWMTWIRQAPGKGL
EWIASITKTGGGTYYADSVKGRFTISRDNSKNTLYLQMNSLRAED
TAVYYCTTLQQLGVMDAWGQGTLVTVSS
SEQ ID NO: 175 CDR-H1: NYWMT
SEQ ID NO: 176 CDR-H2: SITKTGGGTYYADSVKG
SEQ ID NO: 177 CDR-H3: LQQLGVMDA
Clone #12 Light Chain
Sequence without signal peptide
CDRs
VL (Light chain variable sequence)
LC (Light chain constant sequence)
SEQ ID NO: 178
DIQMTQSPSSLSASVGDRVTITCRASQSINKELNWYQQKPGKAPK
LLIYNTNNLQSGVPSRFSGSGSGTDFTLTISSLQPEDFATYYCFQ
HKSWPLTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 179
DIQMTQSPSSLSASVGDRVTITCRASQSINKELNWYQQKPGKAPK
LLIYNTNNLQSGVPSRFSGSGSGTDFTLTISSLQPEDFATYYCFQ
HKSWPLTFGQGTKLEIK
SEQ ID NO: 180 CDR-L1: RASQSINKELN
SEQ ID NO: 181 CDR-L2: NTNNLQS
SEQ ID NO: 182 CDR-L3: FQHKSWPLT
Clone #13
(VH3 + VK1; HC-LALA = same as Clone #3
but with LALA mutation in HC constant region),
preferred clone/embodiment
Clone #13 Heavy Chain
Sequence without signal peptide
CDRs
VH (Heavy chain variable sequence)
HC (Heavy chain constant sequence)
AA-LALA mutations
SEQ ID NO: 183
QVQLVESGGGVVQPGRSLRLSCAASGFTFNNYWMTWVRQAPGKGL
EWIASITKTGGGTYYADSVKGRFTISRDNSKNTLYLQMNSLRAED
TAVYYCTTLQQLGVMDAWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPEAAGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 184
QVQLVESGGGVVQPGRSLRLSCAASGFTFNNYWMTWVRQAPGKGL
EWIASITKTGGGTYYADSVKGRFTISRDNSKNTLYLQMNSLRAED
TAVYYCTTLQQLGVMDAWGQGTLVTVSS
SEQ ID NO: 185 CDR-H1: NYWMT
SEQ ID NO: 186 CDR-H2: SITKTGGGTYYADSVKG
SEQ ID NO: 187 CDR-H3: LQQLGVMDA
Clone #13 Light Chain
Sequence without signal peptide
CDRs
VL (Light chain variable sequence)
LC (Light chain constant sequence)
SEQ ID NO: 188
DIQMTQSPSVLSASVGDRVTINCRASQNINKELNWYQQKLGEAPK
LLIYNTNNLQSGVPSRFSGSGSGTDYTLTISSLQPEDVATYFCFQ
HKSWPLTFGSGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
SEQ ID NO: 189
DIQMTQSPSVLSASVGDRVTINCRASQNINKELNWYQQKLGEAPK
LLIYNTNNLQSGVPSRFSGSGSGTDYTLTISSLQPEDVATYFCFQ
HKSWPLTFGSGTKLEIK
SEQ ID NO: 190 CDR-L1: RASQNINKELN
SEQ ID NO: 191 CDR-L2: NTNNLQS
SEQ ID NO: 192 CDR-L3: FQHKSWPLT
Exemplary non-limiting heavy chain signal sequence
SEQ ID NO: 193 MDWTWRILFLVAAATGAHS
Exemplary non-limiting light chain signal sequence
SEQ ID NO: 194 MLPSQLIGFLLLWVPASRG
LC (Light chain constant sequence)
Human Immunoglobulin kappa constant
HC (Heavy chain constant sequence)
Immunoglobulin heavy constant gamma
For clones #1-12: wild-type
For clone #13: LALA mutant of clone #3

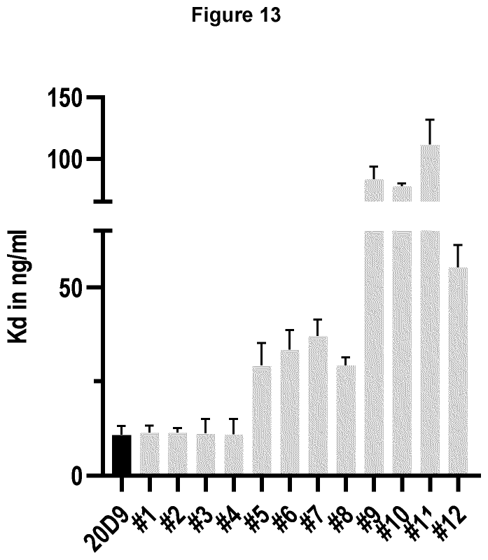

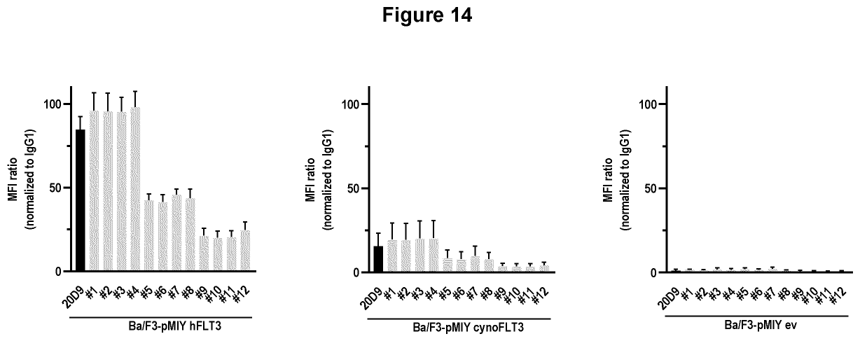

The 12 clones were subsequently analysed in ELISA assays (FIG. 13). Therefore, recombinant human FLT3 was coated on plates and incubated with the antibody candidates. In this assay, clone #1-#4 showed the highest affinity with EC50 values around 10 ng/ml, similar to the affinity of the 20D9 original chimeric clone. Binding of humanized antibody clones to human FLT3 was carried out as shown in FIG. 13, according to which binding was assessed by ELISA assay with recombinant human FLT3 coated to ELISA plates; The calculation of the dissociation constant Kd was performed using GraphPad Prism. Mean±s.d. of n=3 biological replicates. These results were further confirmed in FACS assays, where saturating concentrations of the antibodies were incubated with murine BaF3 cells expressing human or cynomolgous FLT3 (FIG. 14). Similarly, the antibody candidates #1-#4 showed the highest binding, which was even slightly higher compared to the parental 20D9 (FIG. 14). Accordingly, binding of humanized antibody clones to human FLT3 is shown in FIG. 14, according to which cell surface binding of humanized mabs to Ba/F3 cells stably expressing pMIY (Ba/F3-PMIY) empty vector (ev), human wildtype (hFLT3 wt) or cynomolgous FLT3 (cynoFLT3) was measured in flow cytometry and normalized to binding of control IgG1 antibody; Mean±s.d. of n=3.

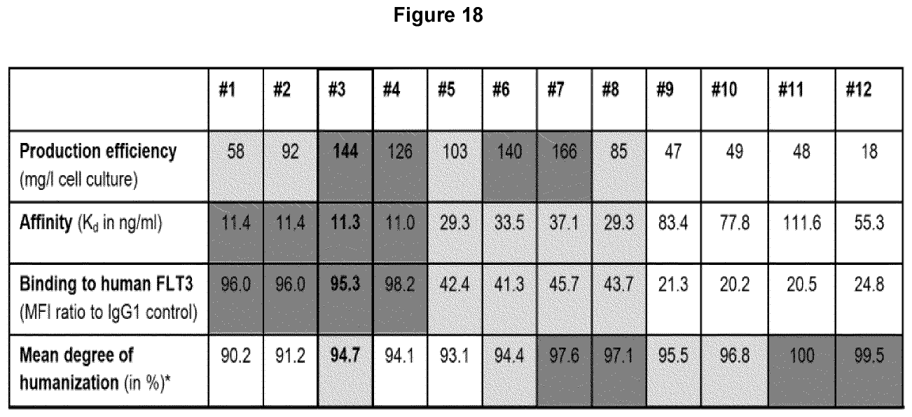

FIG. 18 summarizes these results. Based on this candidate #3—with the highest degree of humanization—was chosen as lead candidate for further analysis.

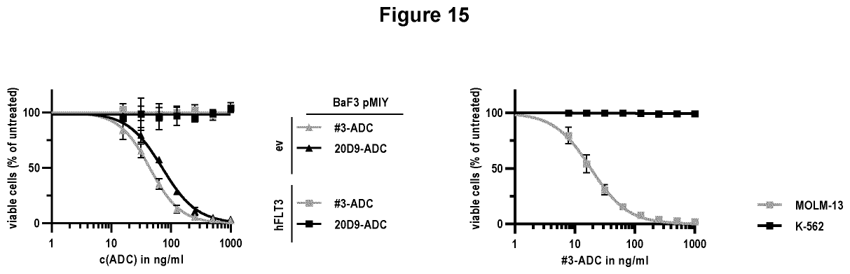

Candidate #3 was subsequently conjugated with MMAF and subjected to cytotoxicity assays using hFLT3 expressing BaF3 cells as well as MOLM-13 (FLT3-positive) and K-562 (FLT3-negative) cells. In these assays, #3-MMAF-ADC showed FLT3-specific and efficient killing with even slightly increased efficacy over 20D9-ADC. Accordingly, here the improved cytotoxicity of the favored #3 as MMAF-ADC is shown (FIG. 15).

Comparison of #3-ADC and 20D9-ADC cytotoxicity is shown in FIG. 15, according to which assessment of cytotoxicity of ADCs in BaF3 cell model expressing empty vector (ev) or human FLT3 (hFLT3) and different cell lines was carried out; viability was determined after 96h treatment with different concentrations of ADCs by resazurin fluorescence and normalized to untreated control; Mean±s.d. of n=3 biological replicates. It would be expected, that the humanization worsence the activity on an ADC level due to the alterations made in the original chimeric sequence, resulting in reduced binding and internalization, for instance for Clone 3 vs. 20D9. Surprisingly, the ADC from Clone number 3, conjugated to P5-VC-PAB-MMAF, as shown in figure E2, showed a stronger selective cell killing, compared to the parental 20D9-ADC, carrying the same MMAF linker-payload.

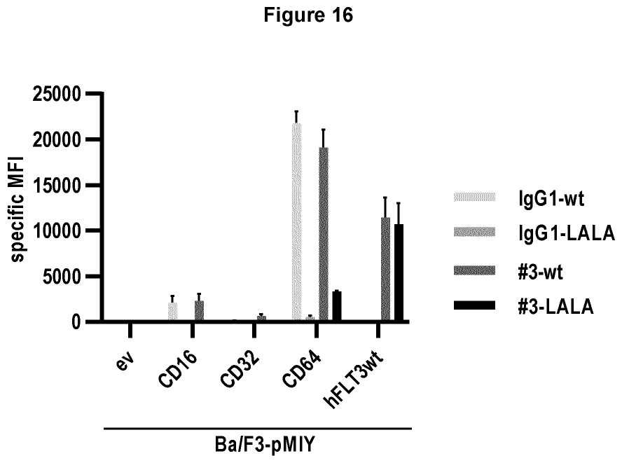

Next, clone #3 was mutated with a Leu234Ala/Leu235Ala (LALA)-mutation in the Fc region. This mutation leads to a reduction of antibody effector functions and specifically to a reduced binding of the antibody to the Fcγ receptor I-III (e.g., CD16, CD32 and CD64). In a FACS binding assay with Fcγ receptor expressing BaF3 cells we could indeed show that in the LALA mutants (#3-LALA) binding to CD16 is abolished and binding to CD64 is greatly reduced. Binding to CD32 was already low in antibodies without LALA mutation and completely abolished in antibodies with LALA mutation (FIG. 16). According, we have produced clone #3 still as a LALA variant and show that the binding properties to FLT3 do not change, but those to the FC receptors do (silencing). Analysis of the effect of LALA mutation on binding of Fcγ receptors is shown in FIG. 16, according to which cell surface binding of #3 and #3-LALA and control IgG1 and IgG1-LALA mab to Ba/F3 cells stably expressing pMIY (Ba/F3-pMIY) empty vector (ev), human wildtype (hFLT3 wt) or Fcγ receptors (CD16, CD32, CD64) was measured in flow cytometry; Mean±s.d. of n=3.

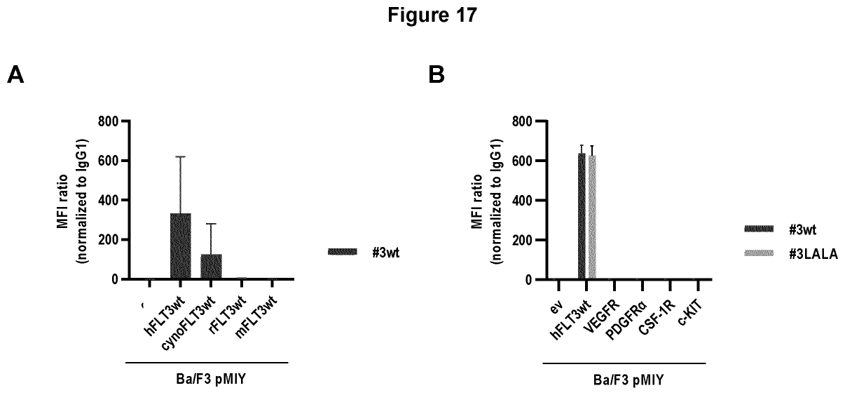

Further we examined binding to FLT3 orthologues from mouse and rat and to FLT3 structural homologues VEGFR-2, PDGFRα, c-KIT and CSF1R. #3 exclusively binds to FLT3 from human and cynomolgous monkey and does not bind rat and mouse FLT3 or structurally similar type III RTKs (FIG. 17). Analysis of #3 mab binding to FLT3 orthologues and homologues is shown in FIG. 17, according to which cell surface binding of #3 and #3-LALA mab to Ba/F3 cells stably expressing pMIY (Ba/F3-pMIY) empty vector (ev), human wildtype (hFLT3 wt) and FLT3 orthologues from mouse (mFLT3), cynomolgous monkey (cynoFLT3) and rat (ratFLT3) (A) or FLT3 homologues (VEGFR, PDGFRα, CSF-1R, c-KIT, (B)) was measured in flow cytometry; Mean±s.d. of n=3.

Based on the above, the antibody candidates #3 and #3-LALA were chosen as lead candidates in example 3 and therefore are preferred embodiments of the present invention.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. An anti-FLT3 antibody (Receptor-type tyrosine-protein kinase FLT3), wherein said anti-FLT3 antibody is capable of the following:
a) binding to an extracellular domain of said FLT3;
b) cross-reactivity with cynomolgus monkey (e.g., Macaca fascicularis) FLT3 (e.g., having UniProtKB Accession Number: Q95M30);
c) internalization, preferably by the means of the antigen-mediated antibody internalization;
d) binding to an extracellular epitope of said FLT3, wherein said extracellular epitope comprising one or more of the following sequences:
i) SEQ ID NO: 2 (Epitope 1), preferably further comprising SEQ ID NO: 4 (Epitope 3); and/or
ii) SEQ ID NO: 3 (Epitope 2), preferably further comprising SEQ ID NO: 4 (Epitope 3);
e) preferably said FLT3 is a human FLT3 having SEQ ID NO: 1 or UniProtKB Accession Number: P36888, further preferably said extracellular domain ranges from amino acids 27 to 543 of SEQ ID NO: 1.

2. The antibody of claim 1, wherein said antibody is selected from the group consisting of:
(a) an antibody (e.g., 20D9) comprising a heavy chain variable region having an amino acid sequence with at least 80% identity to SEQ ID NO: 5 and a light chain variable region having an amino acid sequence with at least 80% identity to SEQ ID NO: 6; preferably wherein said antibody is capable of binding to FLT3-ITD and/or FLT3-TKD mutated polypeptides;
(b) an antibody (e.g., 2F12) comprising a heavy chain variable region having an amino acid sequence with at least 80% identity to SEQ ID NO: 7 and a light chain variable region having an amino acid sequence with at least 80% identity to SEQ ID NO: 8;
(c) an antibody (e.g., 4B12) comprising a heavy chain variable region having an amino acid sequence with at least 80% identity to SEQ ID NO: 9 and a light chain variable region having an amino acid sequence with at least 80% identity to SEQ ID NO: 10;
(d) an antibody (e.g., 27E7) comprising a heavy chain variable region having an amino acid sequence with at least 80% identity to SEQ ID NO: 11 and a light chain variable region having an amino acid sequence with at least 80% identity to SEQ ID NO: 12;
(e) an antibody (e.g., 29H1) comprising a heavy chain variable region having an amino acid sequence with at least 80% identity to SEQ ID NO: 13 and a light chain variable region having an amino acid sequence with at least 80% identity to SEQ ID NO: 14;
(f) an antibody (e.g., 30B12) comprising a heavy chain variable region having an amino acid sequence with at least 80% identity to SEQ ID NO: 15 and a light chain variable region having an amino acid sequence with at least 80% identity to SEQ ID NO: 16;
(g) an antibody (e.g., 19H5) comprising a heavy chain variable region having an amino acid sequence with at least 80% identity to SEQ ID NO: 17 and a light chain variable region having an amino acid sequence with at least 80% identity to SEQ ID NO: 18.

3. The antibody of claim 1, wherein said antibody is selected from the group consisting of:
(a) an antibody comprising a heavy chain variable region having an amino acid sequence with at least 80% identity to SEQ ID NO: 5 and a light chain variable region having an amino acid sequence with at least 80% identity to SEQ ID NO: 6, wherein said antibody (e.g., 20D9) comprising a heavy chain variable region comprising heavy chain CDR1 having the amino acid sequence as set forth in SEQ ID NO: 45 (NYWMT), heavy chain CDR2 having the amino acid sequence as set forth in SEQ ID NO: 46 (SITKTGGGTYYPDSVKG), and heavy chain CDR3 having the amino acid sequence as set forth in SEQ ID NO: 47 (LQQLGVMDA) and a light chain variable region comprising light chain CDR1 having the amino acid sequence as set forth in SEQ ID NO: 48 (KASQNINKELN), light chain CDR2 having the amino acid sequence as set forth in SEQ ID NO: 49 (NTNNLQT), and light chain CDR3 having the amino acid sequence as set forth in SEQ ID NO: 50 (FQHKSWPLT); preferably wherein said antibody is capable of binding to FLT3-ITD and/or FLT3-TKD mutated polypeptides;
(b) an antibody (e.g., 30B12) comprising a heavy chain variable region having an amino acid sequence with at least 80% identity to SEQ ID NO: 15 and a light chain variable region having an amino acid sequence with at least 80% identity to SEQ ID NO: 16, wherein said antibody (e.g., 30B12) comprising a heavy chain variable region comprising heavy chain CDR1 having the amino acid sequence as set forth in SEQ ID NO: 21 (NYHVS), heavy chain CDR2 having the amino acid sequence as set forth in SEQ ID NO: 22 (AISSGGSTYYNSPLKS), and heavy chain CDR3 having the amino acid sequence as set forth in SEQ ID NO: 23 (EDGYTFGNVMDA) and a light chain variable region comprising light chain CDR1 having the amino acid sequence as set forth in SEQ ID NO: 24 (KSSQSLLYSDGKTYLN), light chain CDR2 having the amino acid sequence as set forth in SEQ ID NO: 25 (LVSKLDS), and light chain CDR3 having the amino acid sequence as set forth in SEQ ID NO: 26 (WQGTHFPYT);
(c) an antibody (e.g., 2F12) comprising a heavy chain variable region having an amino acid sequence with at least 80% identity to SEQ ID NO: 7 and a light chain variable region having an amino acid sequence with at least 80% identity to SEQ ID NO: 8, wherein said antibody (e.g., 2F12) comprising a heavy chain variable region comprising heavy chain CDR 1 having the amino acid sequence as set forth in SEQ ID NO: 27 (NYWMN), heavy chain CDR2 having the amino acid sequence as set forth in SEQ ID NO: 28 (QIRLKSDNYATHYAESVKG), and heavy chain CDR3 having the amino acid sequence as set forth in SEQ ID NO: 29 (SLARSDY) and a light chain variable region comprising light chain CDR1 having the amino acid sequence as set forth in SEQ ID NO: 30 (QASQNINKYIA), light chain CDR2 having the amino acid sequence as set forth in SEQ ID NO: 31 (YTSTLES), and light chain CDR3 having the amino acid sequence as set forth in SEQ ID NO: 32 (LQYVNLPRT);
(d) an antibody (e.g., 4B12) comprising a heavy chain variable region having an amino acid sequence with at least 80% identity to SEQ ID NO: 9 and a light chain variable region having an amino acid sequence with at least 80% identity to SEQ ID NO: 10, wherein said antibody (e.g., 4B12) comprising a heavy chain variable region comprising heavy chain CDR 1 having the amino acid sequence as set forth in SEQ ID NO: 33 (SYNVN), heavy chain CDR2 having the amino acid sequence as set forth in SEQ ID NO: 34 (AMWRGGGTDYNPALKS), and heavy chain CDR3 having the amino acid sequence as set forth in SEQ ID NO: 35 (GFGDY) and a light chain variable region comprising light chain CDR1 having the amino acid sequence as set forth in SEQ ID NO: 36 (KSSQSLKYSDGKTYLN), light chain CDR2 having the amino acid sequence as set forth in SEQ ID NO: 37 (QVSKLDS), and light chain CDR3 having the amino acid sequence as set forth in SEQ ID NO: 38 (CQGSYSPST);
(e) an antibody (e.g., 19H5) comprising a heavy chain variable region having an amino acid sequence with at least 80% identity to SEQ ID NO: 17 and a light chain variable region having an amino acid sequence with at least 80% identity to SEQ ID NO: 18, wherein said antibody (e.g., 19H5) comprising a heavy chain variable region comprising heavy chain CDR1 having the amino acid sequence as set forth in SEQ ID NO: 39 (SYNVN), heavy chain CDR2 having the amino acid sequence as set forth in SEQ ID NO: 40 (AMWRGGGTDYNPALKS), and heavy chain CDR3 having the amino acid sequence as set forth in SEQ ID NO: 41 (GFGDY) and a light chain variable region comprising light chain CDR1 having the amino acid sequence as set forth in SEQ ID NO: 42 (QASQDIGNNLI), light chain CDR2 having the amino acid sequence as set forth in SEQ ID NO: 43 (CATNLAH), and light chain CDR3 having the amino acid sequence as set forth in SEQ ID NO: 44 (LQYEHYPRT);
(f) an antibody (e.g., 27E7) comprising a heavy chain variable region having an amino acid sequence with at least 80% identity to SEQ ID NO: 11 and a light chain variable region having an amino acid sequence with at least 80% identity to SEQ ID NO: 12, wherein said antibody (e.g., 27E7) comprising a heavy chain variable region comprising heavy chain CDR1 having the amino acid sequence as set forth in SEQ ID NO: 51 (NYWMN), heavy chain CDR2 having the amino acid sequence as set forth in SEQ ID NO: 52 (QIRLKSDNYATHYAESVKG), and heavy chain CDR3 having the amino acid sequence as set forth in SEQ ID NO: 53 (SLARSDY) and a light chain variable region comprising light chain CDR1 having the amino acid sequence as set forth in SEQ ID NO: 54 (KSSQSLLYSDGKTYLN), light chain CDR2 having the amino acid sequence as set forth in SEQ ID NO: 55 (LVSKLDS), and light chain CDR3 having the amino acid sequence as set forth in SEQ ID NO: 56 (WQGTHFPYT);
(g) an antibody (e.g., 29H1) comprising a heavy chain variable region having an amino acid sequence with at least 80% identity to SEQ ID NO: 13 and a light chain variable region having an amino acid sequence with at least 80% identity to SEQ ID NO: 14, wherein said antibody (e.g., 29H1) comprising a heavy chain variable region comprising heavy chain CDR1 having the amino acid sequence as set forth in SEQ ID NO: 57 (NYWMN), heavy chain CDR2 having the amino acid sequence as set forth in SEQ ID NO: 58 (QIKLKSDNYATRYAESVKG), and heavy chain CDR3 having the amino acid sequence as set forth in SEQ ID NO: 59 (SLARSDY) and a light chain variable region comprising light chain CDR1 having the amino acid sequence as set forth in SEQ ID NO: 60 (KSSQSLLHSDGKTYLN), light chain CDR2 having the amino acid sequence as set forth in SEQ ID NO: 61 (LVSKLDS), and light chain CDR3 having the amino acid sequence as set forth in SEQ ID NO: 62 (WQGTHFPYT).

4. The antibody of claim 1, wherein said antibody is a humanized antibody, wherein said antibody is selected from the group consisting of:
i) an antibody comprising a heavy chain variable region comprising heavy chain CDR1 having the amino acid sequence as set forth in SEQ ID NO: 65, heavy chain CDR2 having the amino acid sequence as set forth in SEQ ID NO: 66, and heavy chain CDR3 having the amino acid sequence as set forth in SEQ ID NO: 67 and a light chain variable region comprising light chain CDR1 having the amino acid sequence as set forth in SEQ ID NO: 70, light chain CDR2 having the amino acid sequence as set forth in SEQ ID NO: 71, and light chain CDR3 having the amino acid sequence as set forth in SEQ ID NO: 72;
ii) an antibody comprising a heavy chain variable region comprising heavy chain CDR1 having the amino acid sequence as set forth in SEQ ID NO: 75, heavy chain CDR2 having the amino acid sequence as set forth in SEQ ID NO: 76, and heavy chain CDR3 having the amino acid sequence as set forth in SEQ ID NO: 77 and a light chain variable region comprising light chain CDR1 having the amino acid sequence as set forth in SEQ ID NO: 80, light chain CDR2 having the amino acid sequence as set forth in SEQ ID NO: 81, and light chain CDR3 having the amino acid sequence as set forth in SEQ ID NO: 82;
iii) an antibody comprising a heavy chain variable region comprising heavy chain CDR1 having the amino acid sequence as set forth in SEQ ID NO: 85, heavy chain CDR2 having the amino acid sequence as set forth in SEQ ID NO: 86, and heavy chain CDR3 having the amino acid sequence as set forth in SEQ ID NO: 87 and a light chain variable region comprising light chain CDR1 having the amino acid sequence as set forth in SEQ ID NO: 90, light chain CDR2 having the amino acid sequence as set forth in SEQ ID NO: 91, and light chain CDR3 having the amino acid sequence as set forth in SEQ ID NO: 92;
iv) an antibody comprising a heavy chain variable region comprising heavy chain CDR1 having the amino acid sequence as set forth in SEQ ID NO: 95, heavy chain CDR2 having the amino acid sequence as set forth in SEQ ID NO: 96, and heavy chain CDR3 having the amino acid sequence as set forth in SEQ ID NO: 97 and a light chain variable region comprising light chain CDR1 having the amino acid sequence as set forth in SEQ ID NO: 100, light chain CDR2 having the amino acid sequence as set forth in SEQ ID NO: 101, and light chain CDR3 having the amino acid sequence as set forth in SEQ ID NO: 102;
v) an antibody comprising a heavy chain variable region comprising heavy chain CDR1 having the amino acid sequence as set forth in SEQ ID NO: 105, heavy chain CDR2 having the amino acid sequence as set forth in SEQ ID NO: 105, and heavy chain CDR3 having the amino acid sequence as set forth in SEQ ID NO: 107 and a light chain variable region comprising light chain CDR1 having the amino acid sequence as set forth in SEQ ID NO: 110, light chain CDR2 having the amino acid sequence as set forth in SEQ ID NO: 111, and light chain CDR3 having the amino acid sequence as set forth in SEQ ID NO: 112;
vi) an antibody comprising a heavy chain variable region comprising heavy chain CDR1 having the amino acid sequence as set forth in SEQ ID NO: 115, heavy chain CDR2 having the amino acid sequence as set forth in SEQ ID NO: 116, and heavy chain CDR3 having the amino acid sequence as set forth in SEQ ID NO: 117 and a light chain variable region comprising light chain CDR1 having the amino acid sequence as set forth in SEQ ID NO: 120, light chain CDR2 having the amino acid sequence as set forth in SEQ ID NO: 121, and light chain CDR3 having the amino acid sequence as set forth in SEQ ID NO: 122;
vii) an antibody comprising a heavy chain variable region comprising heavy chain CDR1 having the amino acid sequence as set forth in SEQ ID NO: 125, heavy chain CDR2 having the amino acid sequence as set forth in SEQ ID NO: 126, and heavy chain CDR3 having the amino acid sequence as set forth in SEQ ID NO: 127 and a light chain variable region comprising light chain CDR1 having the amino acid sequence as set forth in SEQ ID NO: 130, light chain CDR2 having the amino acid sequence as set forth in SEQ ID NO: 131, and light chain CDR3 having the amino acid sequence as set forth in SEQ ID NO: 132;
viii) an antibody comprising a heavy chain variable region comprising heavy chain CDR1 having the amino acid sequence as set forth in SEQ ID NO: 135, heavy chain CDR2 having the amino acid sequence as set forth in SEQ ID NO: 136, and heavy chain CDR3 having the amino acid sequence as set forth in SEQ ID NO: 137 and a light chain variable region comprising light chain CDR1 having the amino acid sequence as set forth in SEQ ID NO: 140, light chain CDR2 having the amino acid sequence as set forth in SEQ ID NO: 141, and light chain CDR3 having the amino acid sequence as set forth in SEQ ID NO: 142;
ix) an antibody comprising a heavy chain variable region comprising heavy chain CDR1 having the amino acid sequence as set forth in SEQ ID NO: 145, heavy chain CDR2 having the amino acid sequence as set forth in SEQ ID NO: 146, and heavy chain CDR3 having the amino acid sequence as set forth in SEQ ID NO: 147 and a light chain variable region comprising light chain CDR1 having the amino acid sequence as set forth in SEQ ID NO: 150, light chain CDR2 having the amino acid sequence as set forth in SEQ ID NO: 151, and light chain CDR3 having the amino acid sequence as set forth in SEQ ID NO: 152;
x) an antibody comprising a heavy chain variable region comprising heavy chain CDR1 having the amino acid sequence as set forth in SEQ ID NO: 155, heavy chain CDR2 having the amino acid sequence as set forth in SEQ ID NO: 156, and heavy chain CDR3 having the amino acid sequence as set forth in SEQ ID NO: 157 and a light chain variable region comprising light chain CDR1 having the amino acid sequence as set forth in SEQ ID NO: 160, light chain CDR2 having the amino acid sequence as set forth in SEQ ID NO: 161, and light chain CDR3 having the amino acid sequence as set forth in SEQ ID NO: 162;
xi) an antibody comprising a heavy chain variable region comprising heavy chain CDR1 having the amino acid sequence as set forth in SEQ ID NO: 165, heavy chain CDR2 having the amino acid sequence as set forth in SEQ ID NO: 166, and heavy chain CDR3 having the amino acid sequence as set forth in SEQ ID NO: 167 and a light chain variable region comprising light chain CDR1 having the amino acid sequence as set forth in SEQ ID NO: 170, light chain CDR2 having the amino acid sequence as set forth in SEQ ID NO: 171, and light chain CDR3 having the amino acid sequence as set forth in SEQ ID NO: 172;
xii) an antibody comprising a heavy chain variable region comprising heavy chain CDR1 having the amino acid sequence as set forth in SEQ ID NO: 175, heavy chain CDR2 having the amino acid sequence as set forth in SEQ ID NO: 176, and heavy chain CDR3 having the amino acid sequence as set forth in SEQ ID NO: 177 and a light chain variable region comprising light chain CDR1 having the amino acid sequence as set forth in SEQ ID NO: 180, light chain CDR2 having the amino acid sequence as set forth in SEQ ID NO: 181, and light chain CDR3 having the amino acid sequence as set forth in SEQ ID NO: 182;
xiii) an antibody comprising a heavy chain variable region comprising heavy chain CDR1 having the amino acid sequence as set forth in SEQ ID NO: 185, heavy chain CDR2 having the amino acid sequence as set forth in SEQ ID NO: 186, and heavy chain CDR3 having the amino acid sequence as set forth in SEQ ID NO: 187 and a light chain variable region comprising light chain CDR1 having the amino acid sequence as set forth in SEQ ID NO: 190, light chain CDR2 having the amino acid sequence as set forth in SEQ ID NO: 191, and light chain CDR3 having the amino acid sequence as set forth in SEQ ID NO: 192.

5. The antibody of claim 1, wherein said antibody is selected from the group consisting of:
(a) an antibody comprising a heavy chain comprising SEQ ID NO: 63 and a light chain comprising SEQ ID NO: 68;
(b) an antibody comprising a heavy chain comprising SEQ ID NO: 73 and a light chain comprising SEQ ID NO: 78;
(c) an antibody comprising a heavy chain comprising SEQ ID NO: 83 and a light chain comprising SEQ ID NO: 88;
(d) an antibody comprising a heavy chain comprising SEQ ID NO: 93 and a light chain comprising SEQ ID NO: 98;
(e) an antibody comprising a heavy chain comprising SEQ ID NO: 103 and a light chain comprising SEQ ID NO: 108;
(f) an antibody comprising a heavy chain comprising SEQ ID NO: 113 and a light chain comprising SEQ ID NO: 118;
(g) an antibody comprising a heavy chain comprising SEQ ID NO: 123 and a light chain comprising SEQ ID NO: 128;
(h) an antibody comprising a heavy chain comprising SEQ ID NO: 133 and a light chain comprising SEQ ID NO: 138;
(i) an antibody comprising a heavy chain comprising SEQ ID NO: 143 and a light chain comprising SEQ ID NO: 148;
(j) an antibody comprising a heavy chain comprising SEQ ID NO: 153 and a light chain comprising SEQ ID NO: 158;
(k) an antibody comprising a heavy chain comprising SEQ ID NO: 163 and a light chain comprising SEQ ID NO: 168;
(l) an antibody comprising a heavy chain comprising SEQ ID NO: 173 and a light chain comprising SEQ ID NO: 178;
(m) an antibody comprising a heavy chain comprising SEQ ID NO: 183 and a light chain comprising SEQ ID NO: 188.

6. The antibody of claim 1, wherein said antibody having one or more of the following characteristics:
(i) a monoclonal antibody;
(ii) a chimeric antibody and/or humanized antibody;
(iii) having K_{D} in the range from about 0.1 ng/ml to about 4000 ng/ml, preferably from about 10 ng/ml to about 4000 ng/ml, further preferably in the range from about 11.5 ng/ml to about 3981 ng/ml;
(iv) capable of being internalized by target cells expressing FLT3, preferably capable of being internalized by said target cells with at least 50% efficiency, further preferably capable of being internalized by said target cells with at least 80% efficiency; most preferably said internalized antibody is directed to endosomes;
(v) a tumor-selective antibody, preferably said tumor is a liquid tumor
(vi) a malignant-cell selective antibody.

7. A hybridoma, wherein said hybridoma produces the monoclonal antibody of claim 1.

8. A nucleic acid encoding the antibody of claim 1.

9. An expression vector comprising at least one of the nucleic acid molecules encoding the antibody of claim 1.

10. An isolated host cell (e.g., an isolated recombinant host cell) comprising the vector comprising at least one of the nucleic acid molecules encoding the antibody of claim 1 and/or nucleic acid encoding said antibody.

11. An antibody drug conjugate (ADC) comprising the anti-FLT3 antibody of claim 1 conjugated via its interchain disulfidebond forming cysteine residues to a phosphonamidate linked, cathepsin B cleavable monomethyl auristatin F (MMAF) cytotoxic drug having Formula I: 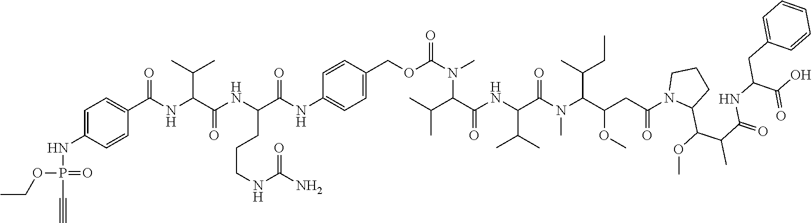 [SMILES: C#CP(=O)(Nc1ccc(C(=O)NC(C(=O)NC(CCCNC(N)=O)C(=O)Nc2ccc(COC(=O)N(C)C(C(=O)NC(C(=O)N(C)C(C(C)CC)C(CC(=O)N3CCCC3C(OC)C(C)C(=O)NC(Cc3ccccc3)C(=O)O)OC)C(C)C)C(C)C)cc2)C(C)C)cc1)OCC]
preferably to form an ADC having Formula III: 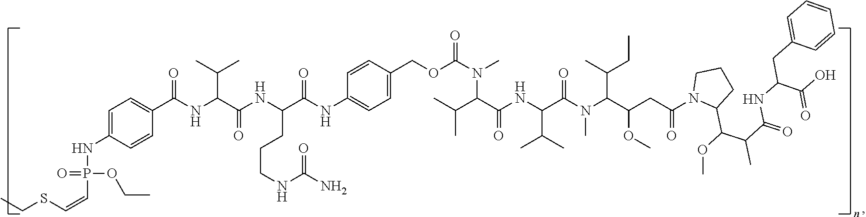
wherein n is in the range from 0 to 20 (e.g., 1, 2, 3, 4, 5, 6, 7 or 8).

12. The antibody drug conjugate (ADC) of claim 11, wherein said ADC is not glycosylated, preferably said ADC is deglycosylated.

13. A composition or kit comprising said anti-FLT3 antibody of claim 1, antibody drug conjugate (ADC) comprising said antibody, hybridoma producing said monoclonal antibody, nucleic acid encoding said antibody, expression vector comprising at least one of the nucleic acid molecules encoding said antibody and/or host cell comprising the vector comprising at least one of the nucleic acid molecules encoding said antibody and/or nucleic acid encoding said antibody.

14. The composition or kit of claim 1, wherein said composition or kit is a pharmaceutical and/or diagnostic composition or kit, wherein said pharmaceutical or diagnostic composition further comprising a receptor tyrosine kinase inhibitor (TKI), preferably said TKI is selected from the group consisting of: Midostaurin, Sorafenib, Lestaurtinib, Sunitinib, Quizartinib, Crenolanib and Gilteritinib.

15. A method for treatment, amelioration, prophylaxis and/or diagnostics of cancer, preferably said cancer is acute myeloid leukemia (AML), further preferably said AML comprising one or more activating mutation/s in FLT3 (e.g. FLT3-ITD), said method comprising: administering a therapeutically or prophylactically effective amount of the antibody of claim 1, antibody drug conjugate (ADC) comprising said antibody, nucleic acid encoding said antibody, expression vector comprising at least one of the nucleic acid molecule encoding said antibody, host cell comprising the vector comprising at least one of the nucleic acid molecules encoding said antibody and/or nucleic acid encoding said antibody, composition or kit comprising: said antibody, the antibody drug conjugate (ADC) comprising said antibody the vector comprising at least one of the nucleic acid molecules encoding said antibody and/or nucleic acid encoding said antibody, preferably said ADC is administered at a daily dosage of about ≤14 mg/kg, further preferably at a daily dosage of about 12 mg/kg, 10 mg/kg, 9 mg/kg, 8 mg/kg, 7 mg/kg, 6 mg/kg, 5 mg/kg, 4 mg/kg, 3 mg/kg, 2 mg/kg or 1 mg/kg, most preferably said ADC is administered at a daily dosage of 1-6 mg/kg.

16. The antibody of claim 1, antibody drug conjugate (ADC) comprising said antibody, nucleic acid encoding said antibody, expression vector comprising at least one of the nucleic acid molecule encoding said antibody, host cell comprising the vector comprising at least one of the nucleic acid molecules encoding said antibody and/or nucleic acid encoding said antibody, composition or kit comprising: said antibody, the antibody drug conjugate (ADC) comprising said antibody, the vector comprising at least one of the nucleic acid molecules encoding said antibody and/or nucleic acid encoding said antibody, for use as a medicament and/or in therapy.

17. The antibody of claim 1, antibody drug conjugate (ADC) comprising said antibody, nucleic acid encoding said antibody, expression vector comprising at least one of the nucleic acid molecule encoding said antibody, host cell comprising the vector comprising at least one of the nucleic acid molecules encoding said antibody and/or nucleic acid encoding said antibody, composition or kit comprising: said antibody, the antibody drug conjugate (ADC) comprising said antibody, the vector comprising at least one of the nucleic acid molecules encoding said antibody and/or nucleic acid encoding said antibody, for use in one or more of the following methods:
(a) method for treatment, amelioration, prophylaxis and/or diagnostics of cancer, preferably said cancer is acute myeloid leukemia (AML), further preferably said AML comprising one or more activating mutation/s in FLT3 (FLT3-ITD AML);
(b) method for monitoring development of cancer and/or for assessing the efficacy of cancer therapy;
(c) method for screening a candidate compound for anti-cancer activity;
(d) method for altering resistance of cancer cells to chemotherapy;
(e) method for sensitizing cancer cells to chemotherapy;
(f) method for inhibiting the growth of cancer cell expressing FLT3;
(g) method for production or preparation of an antibody;
(h) method for immunizing a non-human animal;
(i) method for preparation of a hybridoma;
(j) method for treatment, amelioration, prophylaxis and/or diagnostics of cancer, preferably said cancer is acute myeloid leukemia (AML), further preferably said AML comprising one or more activating mutation/s in FLT3 (e.g. FLT3-ITD);
(k) method according to any one of (a)-(j), wherein said method is an in vivo, in vitro, or ex vivo method.

18. A method for one or more of the following:
(a) for treatment, amelioration, prophylaxis and/or diagnostics of cancer, preferably said cancer is acute myeloid leukemia (AML), further preferably said AML comprising internal tandem duplication (ITD) mutations in FLT3 (FLT3-ITD AML);
(b) for monitoring development of cancer and/or for assessing the efficacy of cancer therapy;
(c) for screening a candidate compound for anti-cancer activity;
(d) for altering resistance of cancer cells to chemotherapy;
(e) for sensitizing cancer cells to chemotherapy;
(f) for inhibiting the growth of cancer cell expressing FLT3;
(g) for production or preparation of an antibody;
(h) for immunizing a non-human animal;
(i) for preparation of a hybridoma;
(j) for use according to any one of (a)-(i), wherein said use is an in vivo, in vitro, or ex vivo use;
said method comprising using the antibody of claim 1, antibody drug conjugate (ADC) comprising said antibody, nucleic acid encoding said antibody, expression vector comprising at least one of the nucleic acid molecules encoding said antibody, host cell comprising: the vector comprising at least one of the nucleic acid molecules encoding said antibody and/or nucleic acid encoding said antibody composition or kit comprising: said antibody, the antibody drug conjugate (ADC) comprising said antibody, the vector comprising at least one of the nucleic acid molecules encoding said antibody and/or nucleic acid encoding said antibody.
